SEQUENCE LISTING (44 sequences)

SEQ ID NO: 1 - DNA, 1383 bp, Homo sapiens
atgcttggaatttggattgttgcattcttgttctttggcacatcaagaggaaaagaagtttgctatgaaaggttagggtgtttcaaagatggtttaccatggaccaggactttctcaacagagttggtaggtttaccctggtctccagagaagataaacactcgtttcctgctctacactatacacaatcccaatgcctatcaggagatcagtgcggttaattcttcaactatccaagcctcatattttggaacagacaagatcacccgtatcaacatagctggatggaaaacagatggcaaatggcagagagacatgtgcaatgtgttgctacagctggaagatataaattgcattaatttagattggatcaacggttcacgggaatacatccatgctgtaaacaatctccgtgttgttggtgctgaggtggcttattttattgatgttctcatgaaaaaatttgaatattccccttctaaagtgcacttgattggccacagcttgggagcacacctggctggggaagctgggtcaaggataccaggccttggaagaataactgggttggacccagctgggccatttttccacaacactccaaaggaagtcaggctagacccctcggatgccaactttgttgacgttattcatacaaatgcagctcgcatcctctttgagcttggtgttggaaccattgatgcttgtggtcatcttgacttttacccaaatggagggaagcacatgccaggatgtgaagacttaattacacctttactgaaatttaacttcaatgcttacaaaaaagaaatggcttccttctttgactgtaaccatgcccgaagttatcaattttatgctgaaagcattcttaatcctgatgcatttattgcttatccttgtagatcctacacatcttttaaagcaggtacatgtgtaggatgtgcagatttgttacataggatagataagataggaagtcatacttcccatgtgtttttaaccctttctctccctttccttcttgtttccttatatctaggttggaggcacaaattgtctgttaaactcagtggaagcgaagtcactcaaggaactgtctttcttcgtgtaggcggggcagttaggaaaactggggagtttgccattgtcagtggaaaacttgagccaggcatgacttacacaaaattaatcgatgcagatgttaacgttggaaacattacaagtgttcagttcatctggaaaaaacatttgtttgaagattctcagaataagttgggagcagaaatggtgataaatacatctgggaaatatggatataaatctaccttctgtagccaagacattatgggacctaatattctccagaacctgaaaccatgc

SEQ ID NO: 2 - DNA, 927 bp, Homo sapiens
atgccgttcctgcagctgaaagggagagcaacacctccatcctggagacacgatagccgctcacttgttcacctgctggacggcaaggagggcgtgtgggacaccacgggctatgccttagggagcagagaatcattgaatcctgacatggggattggtgacccacatggacacagcactgtccacaccagggaagcaggcactgcctgtccattacagcttctaggtgctcgggaggccagtctgctggcctgtgggatctgccaggcctctggccaaatcttcatcacccaaaccctggggatcaagggatatcggactgtcgtggccctggataaggtccctgaggatgttcaggaatacagctggtactggggtgcaaacgacagcgcaggaaacatgattatcagccacaaaccgcccagtgcccagcagcctgggcccatgtacactggcagggagagagtgaacagagaaggcagcctgttgatcaggccgactgcattaaatgacacgggaaactacactgttcgggtggttgcaggcaatgagacccaaagagcaaccggctggctggaggttctagatgggcccgactatgtgctgctgaggagcaatcctgatgatttcaacggcattgtgacagctgagatcggctcccaagtggaaatggagtgtatctgctattccttcctggatctcaagtaccactggatccacaatggctccctcctgaacttctcagatgcaaagatgaacctctcgagtcttgcctgggagcagatgggccgttaccgatgcactgtggagaaccccgtgacacagctgatcatgtacatggacgtcaggatccaggccccccatgagtgcagcagctcccctccaggctcatgctttgcacatctccctgcctccatgccctgctag

SEQ ID NO: 3 - DNA, 1374 bp, Homo sapiens
atggacctttccagacccagatggagcctgtggaggagggtcttcctcatggccagtctgctggcctgtgggatctgccaggcctctggccaaatcttcatcacccaaaccctggggatcaagggatatcggactgtcgtggccctggataaggtccctgaggatgttcaggaatacagctggtactggggtgcaaacgacagcgcaggaaacatgattatcagccacaaaccgcccagtgcccagcagcctgggcccatgtacactggcagggagagagtgaacagagaaggcagcctgttgatcaggccgactgcattaaatgacacgggaaactacactgttcgggtggttgcaggcaatgagacccaaagagcaaccggctggctggaggttctagagttgggaagcaatctgggcatctccgtcaatgccagctccctggtggagaacatggattctgtggctgctgactgcctcacaaatgtcaccaacatcacgtggtatgtgaatgatgtgcctacctctagtagtgaccggatgacaatttccccagacggcaagaccctcgtcatcctcagggtcagccgctatgacagaacaattcagtgcatgatagagagtttcccagagatctttcagagaagtgaacgcatctctctgactgtggcctatgggcccgactatgtgctgctgaggagcaatcctgatgatttcaacggcattgtgacagctgagatcggctcccaagtggaaatggagtgtatctgctattccttcctggatctcaagtaccactggatccacaatggctccctcctgaacttctcagatgcaaagatgaacctctcgagtcttgcctgggagcagatgggccgttaccgatgcactgtggagaaccccgtgacacagctgatcatgtacatggacgtcaggatccaggccccccatgagtgtcctcttccttcagggatcttacctgttgtccacagagatttctccatctcaggatccatggtgatgttcctcatcatgctgacagtgctgggtggcgtttacatctgtggagtcctgatccatgctctgatcaaccactactcaatcaggtgccctcattgctctgggacaagggtgggatgttggctgggggctgggactcaggagccagccctccctccagaggggaagcagagccagaaggggagggataagccaggaactaggttgtcagggatcatctggggcagacagatcagcccccaggacctgaagctgatgggagcaagagagggtttagagtcggccatggttctaaatagctgtggggtttcttctagcaacttcccttctctttgtgtttataagggatattaa

SEQ ID NO: 4 - DNA, 2115 bp, Homo sapiens
atgctccatgatgggttgactgcacctgatgggtgtggaatctacagcctgaccgggcgggaagtcctgacgcccttcccaggattgggcactgcggcagccccggcacagggcggggcccacctgaagcagtgtgacctgctgaagctgtcccggcggcagaagcagctctgccggagggagcccggcctggctgagaccctgagggatgctgcgcacctcggcctgcttgagtgccagtttcagttccggcatgagcgctggaactgtagcctggagggcaggatgggcctgctcaagagaggcttcaaagagacagctttcctgtacgcggtgtcctctgccgccctcacccacaccctggcccgggcctgcagcgctgggcgcatggagcgctgcacctgtgatgactctccggggctggagagccggcaggcctggcagtggggcgtgtgcggtgacaacctcaagtacagcaccaagtttctgagcaacttcctggggtccaagagaggaaacaaggacctgcgggcacgggcagacgcccacaatacccacgtgggcatcaaggctgtgaagagtggcctcaggaccacgtgtaagtgccatggcgtatcaggctcctgtgccgtgcgcacctgctggaagcagctctccccgttccgtgagacgggccaggtgctgaaactgcgctatgactcggctgtcaaggtgtccagtgccaccaatgaggccttgggccgcctagagctgtgggcccctgccaggcagggcagcctcaccaaaggcctggccccaaggtctggggacctggtgtacatggaggactcacccagcttctgccggcccagcaagtactcacctggcacagcaggtagggtgtgctcccgggaggccagctgcagcagcctgtgctgcgggcggggctatgacacccagagccgcctggtggccttctcctgccactgccaggtgcagtggtgctgctacgtggagtgccagcaatgtgtgcaggaggagcttgtgtacacctgcaagcactagatgggccctgtggggttcccgaggcagtgccagggagccttctttgagagcagccctgggcagaccagggcccgcctgaccgggcgggaagtcctgacgcccttcccaggattgggcactgcggcagccccggcacagggcggggcccacctgaagcagtgtgacctgctgaagctgtcccggcggcagaagcagctctgccggagggagcccggcctggctgagaccctgagggatgctgcgcacctcggcctgcttgagtgccagtttcagttccggcatgagcgctggaactgtagcctggagggcaggatgggcctgctcaagagaggcttcaaagagacagctttcctgtacgcggtgtcctctgccgccctcacccacaccctggcccgggcctgcagcgctgggcgcatggagcgctgcacctgtgatgactctccggggctggagagccggcaggcctggcagtggggcgtgtgcggtgacaacctcaagtacagcaccaagtttctgagcaacttcctggggtccaagagaggaaacaaggacctgcgggcacgggcagacgcccacaatacccacgtgggcatcaaggctgtgaagagtggcctcaggaccacgtgtaagtgccatggcgtatcaggctcctgtgccgtgcgcacctgctggaagcagctctccccgttccgtgagacgggccaggtgctgaaactgcgctatgactcggctgtcaaggtgtccagtgccaccaatgaggccttgggccgcctagagctgtgggcccctgccaggcagggcagcctcaccaaaggcctggccccaaggtctggggacctggtgtacatggaggactcacccagcttctgccggcccagcaagtactcacctggcacagcaggtagggtgtgctcccgggaggccagctgcagcagcctgtgctgcgggcggggctatgacacccagagccgcctggtggccttctcctgccactgccaggtgcagtggtgctgctacgtggagtgccagcaatgtgtgcaggaggagcttgtgtacacctgcaagcactag

SEQ ID NO: 5 - DNA, 1086 bp, Homo sapiens
atgaagcccctgaggaggccccttcccttcatttgcccctcaccaccatccccaaggctcacctgtctccctcctctcgctctctctagcctgaccgggcgggaagtcctgacgcccttcccaggattgggcactgcggcagccccggcacagggcggggcccacctgaagcagtgtgacctgctgaagctgtcccggcggcagaagcagctctgccggagggagcccggcctggctgagaccctgagggatgctgcgcacctcggcctgcttgagtgccagtttcagttccggcatgagcgctggaactgtagcctggagggcaggatgggcctgctcaagagaggcttcaaagagacagctttcctgtacgcggtgtcctctgccgccctcacccacaccctggcccgggcctgcagcgctgggcgcatggagcgctgcacctgtgatgactctccggggctggagagccggcaggcctggcagtggggcgtgtgcggtgacaacctcaagtacagcaccaagtttctgagcaacttcctggggtccaagagaggaaacaaggacctgcgggcacgggcagacgcccacaatacccacgtgggcatcaaggctgtgaagagtggcctcaggaccacgtgtaagtgccatggcgtatcaggctcctgtgccgtgcgcacctgctggaagcagctctccccgttccgtgagacgggccaggtgctgaaactgcgctatgactcggctgtcaaggtgtccagtgccaccaatgaggccttgggccgcctagagctgtgggcccctgccaggcagggcagcctcaccaaaggcctggccccaaggtctggggacctggtgtacatggaggactcacccagcttctgccggcccagcaagtactcacctggcacagcaggtagggtgtgctcccgggaggccagctgcagcagcctgtgctgcgggcggggctatgacacccagagccgcctggtggccttctcctgccactgccaggtgcagtggtgctgctacgtggagtgccagcaatgtgtgcaggaggagcttgtgtacacctgcaagcactag

SEQ ID NO: 6 - DNA, 1098 bp, Homo sapiens
atgtggctgcttttaacaacaacttgtttgatctgtggaactttaaatgctggtggattccttgatttggaaaatgaagtgaatcctgaggtgtggatgaatactagtgaaatcatcatctacaatggctaccccagtgaagagtatgaagtcaccactgaagatgggtatatactccttgtcaacagaattccttatgggcgaacacatgctaggagcacagcagatgcaggttatgatgtatggatgggaaacagtcggggaaacacttggtcaagaagacacaaaacactctcagagacagatgagaaattctgggcctttagttttgatgaaatggccaaatatgatctcccaggagtaatagacttcattgtaaataaaactggtcaggagaaattgtatttcattggacattcacttggcactacaatagggtttgtagccttttccaccatgcctgaactggcacaaagaatcaaaatgaattttgccttgggtcctacgatctcattcaaatatcccacgggcatttttaccaggttttttctacttccaaattccataatcaaggctgtttttggtaccaaaggtttctttttagaagataagaaaacgaagatagcttctaccaaaatctgcaacaataagatactctggttgatatgtagcgaatttatgtccttatgggctggatccaacaagaaaaatatgaatcagagtcgaatggatgtgtatatgtcacatgctcccactggttcatcagtacacaacattctgcatataaaacagctttaccactctgatgaattcagagcttatgactggggaaatgacgctgataatatgaaacattacaatcagagtcatccccctatatatgacctgactgccatgaaagtgcctactgctatttgggctggtggacatgatgtcctcgtaacaccccaggatgtggccaggatactccctcaaatcaagagtcttcattactttaagctattgccagattggaaccactttgattttgtctggggcctcgatgcccctcaacggatgtacagtgaaatcatagctttaatgaaggcatattcctaa

SEQ ID NO: 7 - DNA, 1194 bp, Homo sapiens
atgtggcagcttttagcagcagcatgctggatgcttcttcttggatctatgtatggttatgacaagaaaggaaacaatgcaaaccctgaagctaatatgaatattagccagattatttcttactggggttatccttatgaagagtatgatgttacaacaaaagatggttatatccttggaatttataggattccacatggaagaggatgcccagggaggacagctccaaagcctgctgtgtatttgcagcatggcttaattgcatctgccagtaactggatttgcaacctgcccaacaacagtttggctttccttctggcagatagtggttatgacgtgtggttggggaacagccgaggaaacacttggtccagaaaacaccttaaattgtcaccgaaatcaccggaatactgggccttcagtttggatgagatggctaaatatgaccttccagccacaatcaattttatcatagagaaaactggacagaagcgactctactacgtgggccactcacaaggcaccaccatagcttttatagcattttctacaaacccagaactggctaaaaagattaagatattttttgcactggctccagttgtcacagttaaatacacccaaagtcctatgaaaaaactaacaaccctttccaggcgagtagttaaggtgttgtttggtgacaaaatgttccaccctcatacattgtttgaccaattcattgccaccaaagtgtgcaatcgaaagctattccgtcgtatttgcagcaacttcctatttactctgagtggatttgatccgcaaaacttaaatatgagtcgcttggatgtttatttgtcacacaatcctgcgggaacatctgttcagaatatgctgcactgggctcagctttaccactctgatgaattcagagcttatgactggggaaatgacgctgataatatgaaacattacaatcagagtcatccccctatatatgacctgactgccatgaaagtgcctactgctatttgggctggtggacatgatgtcctcgtaacaccccaggatgtggccaggatactccctcaaatcaagagtcttcattactttaagctattgccagattggaaccactttgattttgtctggggcctcgatgcccctcaacggatgtacagtgaaatcatagctttaatgaaggcatattcctaa

SEQ ID NO: 8 - DNA, 11118 bp, Homo sapiens
atggcgaagcggctctgcgcggggagcgcactgtgtgttcgcggcccccggggccccgcgccgctgctgctggtcgggctggcgctgctgggcgcggcgcgggcgcgggaggaggcgggcggcggcttcagcctgcacccgccctacttcaacctggccgagggcgcccgcatcgccgcctccgcgacctgcggagaggaggccccggcgcgcggctccccgcgccccaccgaggacctttactgcaagctggtagggggccccgtggccggcggcgaccccaaccagaccatccggggccagtactgtgacatctgcacggctgccaacagcaacaaggcacaccccgcgagcaatgccatcgatggcacggagcgctggtggcagagtccaccgctgtcccgcggcctggagtacaacgaggtcaacgtcaccctggacctgggccaggtcttccacgtggcctacgtcctcatcaagtttgccaactcaccccggccggacctctgggtgctggagcggtccatggacttcggccgcacctaccagccctggcagttctttgcctcctccaagagggactgtctggagcggttcgggccacagacgctggagcgcatcacacgggacgacgcggccatctgcaccaccgagtactcacgcatcgtgcccctggagaacggagagatcgtggtgtccctggtgaacggacgtccgggcgccatgaatttctcctactcgccgctgctacgtgagttcaccaaggccaccaacgtccgcctgcgcttcctgcgtaccaacacgctgctgggccatctcatggggaaggcgctgcgggaccccacggtcacccgccggtattattacagcatcaaggatatcagcatcggaggccgctgtgtctgccacggccacgcggatgcctgcgatgccaaagaccccacggacccgttcaggctgcagtgcacctgccagcacaacacctgcgggggcacctgcgaccgctgctgccccggcttcaatcagcagccgtggaagcctgcgactgccaacagtgccaacgagtgccagtcctgtaactgctacggccatgccaccgactgttactacgaccctgaggtggaccggcgccgcgccagccagagcctggatggcacctatcagggtgggggtgtctgtatcgactgccagcaccacaccaccggcgtcaactgtgagcgctgcctgcccggcttctaccgctctcccaaccaccctctcgactcgccccacgtctgccgccgctgcaactgcgagtccgacttcacggatggcacctgcgaggacctgacgggtcgatgctactgccggcccaacttctctggggagcggtgtgacgtgtgtgccgagggcttcacgggcttcccaagctgctacccgacgccctcgtcctccaatgacaccagggagcaggtgctgccagccggccagattgtgaattgtgactgcagcgcggcagggacccagggcaacgcctgccggaaggacccaagggtgggacgctgtctgtgcaaacccaacttccaaggcacccattgtgagctctgcgcgccagggttctacggccccggctgccagccctgccagtgttccagccctggagtggccgatgaccgctgtgaccctgacacaggccagtgcaggtgccgagtgggcttcgagggggccacatgtgatcgctgtgcccccggctactttcacttccctctctgccagttgtgtggctgcagccctgcaggaaccttgcccgagggctgcgatgaggccggccgctgcctatgccagcctgagtttgctggacctcattgtgaccggtgccgccctggctaccatggtttccccaactgccaagcatgcacctgcgaccctcggggagccctggaccagctctgtggggcgggaggtttgtgccgctgccgccccggctacacaggcactgcctgccaggaatgcagccccggctttcacggcttccccagctgtgtcccctgccactgctctgctgaaggctccctgcacgcagcctgtgacccccggagtgggcagtgcagctgccggccccgtgtgacggggctgcggtgtgacacatgtgtgcccggtgcctacaacttcccctactgcgaagctggctcttgccaccctgccggtctggccccagtggatcctgcccttcctgaggcacaggttccctgtatgtgccgggctcacgtggaggggccgagctgtgaccgctgcaaacctgggttctggggactgagccccagcaaccccgagggctgtacccgctgcagctgcgacctcaggggcacactgggtggagttgctgagtgccagccgggcaccggccagtgcttctgcaagccccacgtgtgcggccaggcctgcgcgtcctgcaaggatggcttctttggactggatcaggctgactattttggctgccgcagctgccggtgtgacattggcggtgcactgggccagagctgtgaaccgaggacgggcgtctgccggtgccgccccaacacccagggccccacctgcagcgagcctgcgagggaccactacctcccggacctgcaccacctgcgcctggagctggaggaggctgccacacctgagggtcacgccgtgcgctttggcttcaaccccctcgagttcgagaacttcagctggaggggctacgcgcagatggcacctgtccagcccaggatcgtggccaggctgaacctgacctcccctgaccttttctggctcgtcttccgatacgtcaaccggggggccatgagtgtgagcgggcgggtctctgtgcgagaggagggcaggtcggccacctgcgccaactgcacagcacagagtcagcccgtggccttcccacccagcacggagcctgccttcatcaccgtgccccagaggggcttcggagagccctttgtgctgaaccctggcacctgggccctgcgtgtggaggccgaaggggtgctcctggactacgtggttctgctgcctagcgcatactacgaggcggcgctcctgcagctgcgggtgactgaggcctgcacataccgtccctctgcccagcagtctggcgacaactgcctcctctacacacacctccccctggatggcttcccctcggccgccgggctggaggccctgtgtcgccaggacaacagcctgccccggccctgccccacggagcagctcagcccgtcgcacccgccactgatcacctgcacgggcagtgatgtggacgtccagcttcaagtggcagtgccacagccaggccgctatgccctagtggtggagtacgccaatgaggatgcccgccaggaggtgggcgtggccgtgcacaccccacagcgggccccccagcaggggctgctctccctgcacccctgcctgtacagcaccctgtgccggggcactgcccgggatacccaggaccacctggctgtcttccacctggactcggaggccagcgtgaggctcacagccgaacaggcacgcttcttcctgcacggggtcactctggtgcccattgaggagttcagcccggagttcgtggagccccgggtcagctgcatcagcagccacggcgcctttggccccaacagtgccgcctgtctgccctcgcgcttcccaaagccgccccagcccatcatcctcagggactgccaggtgatcccgctgccgcccggcctcccgctgacccacgcgcaggatctcactccagccatgtccccagctggaccccgacctcggccccccaccgctgtggaccctgatgcagagcccaccctgctgcgtgagccccaggccaccgtggtcttcaccacccatgtgcccacgctgggccgctatgccttcctgctgcacggctaccagccagcccaccccaccttccccgtggaagtcctcatcaacgccggccgcgtgtggcagggccacgccaacgccagcttctgtccacatggctacggctgccgcaccctggtggtgtgtgagggccaggccctgctggacgtgacccacagcgagctcactgtgaccgtgcgtgtgcccaagggccggtggctctggctggattatgtactcgtggtccctgagaacgtctacagctttggctacctccgggaggagcccctggataaatcctatgacttcatcagccactgcgcagcccagggctaccacatcagccccagcagctcatccctgttctgccgaaacgctgctgcttccctctccctcttctataacaacggagcccgtccatgtggctgccacgaagtaggtgctacaggccccacgtgtgagcccttcgggggccagtgtccctgccatgcccatgtcattggccgtgactgctcccgctgtgccaccggatactggggcttccccaactgcaggccctgtgactgcggtgcccgcctctgtgacgagctcacgggccagtgcatctgcccgccacgcaccatcccgcccgactgcctgctgtgccagccccagacctttggctgccaccccctggtcggctgtgaggagtgtaactgctcagggcccggcatccaggagctcacagaccctacctgtgacacagacagcggccagtgcaagtgcagacccaacgtgactgggcgccgctgtgatacctgctctccgggcttccatggctacccccgctgccgcccctgtgactgtcacgaggcgggcactgcgcctggcgtgtgtgaccccctcacagggcagtgctactgtaaggagaacgtgcagggccccaaatgtgaccagtgcagccttgggaccttctcactggatgctgccaaccccaaaggttgcacccgctgcttctgctttggggccacggagcgctgccggagctcgtcctacacccgccaggagttcgtggatatggagggatgggtgctgctgagcactgaccggcaggtggtgccccacgagcggcagccagggacggagatgctccgtgcagacctgcggcacgtgcctgaggctgtgcccgaggctttccccgagctgtactggcaggccccaccctcctacctgggggaccgggtgtcatcctacggtgggaccctccgttatgaactgcactcagagacccagcggggagatgtctttgtccccatggagagcaggccggatgtggtgctgcagggcaaccagatgagcatcacattcctggagccggcataccccacgcctggccacgttcaccgtgggcagctgcagctggtggaggggaacttccggcatacggagacgcgcaacactgtgtcccgcgaggagctcatgatggtgctggccagcctggagcagctgcagatccgtgccctcttctcacagatctcctcggctgtcttcctgcgcagggtggcactggaggtggccagcccagcaggccagggggccctggccagcaatgtggagctgtgcctgtgccccgccagctaccggggggactcatgccaggaatgtgcccccggcttctatcgggacgtcaaaggtctcttcctgggccgatgtgtcccttgtcagtgccatggacactcagaccgctgcctccctggctctggcgtctgtgtggactgccagcacaacaccgaaggggcccactgtgagcgctgccaggctggcttcgtgagcagcagggacgaccccagcgccccctgtgtcagctgcccctgccccctctcagtgccttccaacaacttcgccgagggctgtgtcctgcgaggcggccgcacccagtgcctctgcaaacctggttatgcaggtgcctcctgcgagcggtgtgcgcccggattctttgggaacccactggtgctgggcagctcctgccagccatgcgactgcagcggcaacggtgaccccaacttgctcttcagcgactgcgaccccctgacgggcgcctgccgtggctgcctgcgccacaccactgggccccgctgcgagatctgtgcccccggcttctacggcaacgccctgctgcccggcaactgcacccggtgcgactgtaccccatgtgggacagaggcctgcgacccccacagcgggcactgcctgtgcaaggcgggcgtgactgggcggcgctgtgaccgctgccaggagggacattttggtttcgatggctgcgggggctgccgcccgtgtgcttgtggaccggccgccgagggctccgagtgccacccccagagcggacagtgccactgccgaccagggaccatgggaccccagtgccgcgagtgtgcccctggctactgggggctccctgagcagggctgcaggcgctgccagtgccctgggggccgctgtgaccctcacacgggccgctgcaactgccccccggggctcagcggggagcgctgcgacacctgcagccagcagcatcaggtgcctgttccaggcgggcctgtgggccacagcatccactgtgaagtgtgtgaccactgtgtggtcctgctcctggatgacctggaacgggccggcgccctcctccccgccattcacgagcaactgcgtggcatcaatgccagctccatggcctgggcccgtctgcacaggctgaacgcctccatcgctgacctgcagagccagctccggagccccctgggcccccgccatgagacggcacagcagctggaggtgctggagcagcagagcacaagcctcgggcaggacgcacggcggctaggcggccaggcaggagccccaagaccccccagggccccgggaggctttcacctgtaccaggcgagccaattgctggccggcaccgaggccacactgggccatgcgaagacgctgttggcggccatccgggctgtggaccgcaccctgagcgagctcatgtcccagacgggccacctggggctggccaatgcctcggctccatcaggtgagcagctgctccggacactggccgaggtggagcggctgctctgggagatgcgggcccgggacctgggggccccgcaggcagcagctgaggctgagttggctgcagcacagagattgctggcccgggtgcaggagcagctgagcagcctctgggaggagaaccaggcactggccacacaaacccgcgaccggctggcccagcacgaggccggcctcatggacctgcgagaggctttgaaccgggcagtggacgccacacgggaggcccaggagctcaacagccgcaaccaggagcgcctggaggaagccctgcaaaggaagcaggagctgtcccgggacaatgccaccctgcaggccactctgcatgcggctagggacaccctggccagcgtcttcagattgctgcacagcctggaccaggctaaggaggagctggagcgcctcgccgccagcctggatggggctcggaccccactgctgcagaggatgcagaccttctccccggcgggcagcaagctgcgtctagtggaggccgccgaggcccacgcacagcagctgggccagctggcactcaatctgtccagcatcatcctggacgtcaaccaggaccgcctcacccagagggccatcgaggcctccaacgcctacagccgcatcctgcaggccgtgcaggctgccgaggatgctgctggccaggccctgcagcaggcggaccacacgtgggcgacggtggtgcggcagggcctggtggaccgagcccagcagctcctggccaacagcactgcactagaagaggccatgctccaggaacagcagaggctgggccttgtgtgggctgccctccagggtgccaggacccagctccgagatgtccgggccaagaaggaccagctggaggcgcacatccaggcggcgcaggccatgcttgccatggacacagacgagacaagcaagaagatcgcacatgccaaggctgtggctgctgaagcccaggacaccgccacccgtgtgcagtcccagctgcaggccatgcaggagaatgtggagcggtggcagggccagtacgagggcctgcggggccaggacctgggccaggcagtgcttgacgcaggccactcagtgtccaccctggagaagacgctgccccagctgctggccaagctgagcatcctggagaaccgtggggtgcacaacgccagcctggccctgtccgccagcattggccgcgtgcgagagctcattgcccaggcccggggggctgccagtaaggtcaaggtgcccatgaagttcaacgggcgctcaggggtgcagctgcgcaccccacgggatcttgccgaccttgctgcctacactgccctcaagttctacctgcagggcccagagcctgagcctgggcagggtaccgaggatcgctttgtgatgtacatgggcagccgccaggccactggggactacatgggtgtgtctctgcgtgacaagaaggtgcactgggtgtatcagctgggtgaggcgggccctgcagtcctaagcatcgatgaggacattggggagcagttcgcagctgtcagcctggacaggactctccagtttggccacatgtccgtcacagtggagagacagatgatccaggaaaccaagggtgacacggtggcccctggggcagaggggctgctcaacctgcggccagacgacttcgtcttctacgtcggggggtaccccagtaccttcacgccccctcccctgcttcgcttccccggctaccggggctgcatcgagatggacacgctgaatgaggaggtggtcagcctctacaacttcgagaggaccttccagctggacacggctgtggacaggccttgtgcccgctccaagtcgaccggggacccgtggctcacggacggctcctacctggacggcaccggcttcgcccgcatcagcttcgacagtcagatcagcaccaccaagcgcttcgagcaggagctgcggctcgtgtcctacagcggggtgctcttcttcctgaagcagcagagccagttcctgtgcttggccgtgcaagaaggcagcctcgtgctgttgtatgactttggggctggcctgaaaaaggccgtcccactgcagcccccaccgcccctgacctcggccagcaaggcgatccaggtgttcctgctggggggcagccgcaagcgtgtgctggtgcgtgtggagcgggccacggtgtacagcgtggagcaggacaatgatctggagctggccgacgcctactacctggggggcgtgccgcccgaccagctgcccccgagcctgcgacggctcttccccaccggaggctcagtccgtggctgcgtcaaaggcatcaaggccctgggcaagtatgtggacctcaagcggctgaacacgacaggcgtgagcgccggctgcaccgccgacctgctggtggggcgcgccatgactttccatggccacggcttccttcgcctggcgctctcgaacgtggcaccgctcactggcaacgtctactccggcttcggcttccacagcgcccaggacagtgccctgctctactaccgggcgtccccggatgggctatgccaggtgtccctgcagcagggccgtgtgagcctacagctcctgaggactgaagtgaaaactcaagcgggcttcgccgatggtgccccccattacgtcgccttctacagcaatgccacgggagtctggctgtatgtcgatgaccagctccagcagatgaagccccaccggggaccaccccccgagctccagccgcagcctgaggggcccccgaggctcctcctgggaggcctgcctgagtctggcaccatttacaacttcagtggctgcatcagcaacgtcttcgtgcagcggctcctgggcccacagcgcgtatttgatctgcagcagaacctgggcagcgtcaatgtgagcacgggctgtgcacccgccctgcaagcccagaccccgggcctggggcctagaggactgcaggccaccgcccggaaggcctcccgccgcagccgtcagcccgcccggcatcctgcctgcatgctgcccccacacctcaggaccacccgagactcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgcatcttgggccccctggaggcgggcctgttcttcccaggcagcgggggagttatcactttagacctcccaggagctacactgcctgatgtgggcctggaactggaggtgcggcccctggcagtcaccggactgatcttccacttgggccaggcccggacgcccccctacttgcagttgcaggtgaccgagaagcaagtcctgctgcgggcggatgacggagcaggggagttctccacgtcagtgacccgcccctcagtgctgtgtgatggccagtggcaccggctagcggtgatgaaaagcgggaatgtgctccggctggaggtggacgcgcagagcaaccacaccgtgggccccttgctggcggctgcagctggtgccccagcccctctgtacctcgggggcctgcctgagcccatggccgtgcagccctggccccccgcctactgcggctgcatgaggaggctggcggtgaaccggtcccccgtcgccatgactcgctctgtggaggtccacggggcagtgggggccagtggctgcccagccgcctag

SEQ ID NO: 9 - DNA, 11091 bp, Homo sapiens
atggcgaagcggctctgcgcggggagcgcactgtgtgttcgcggcccccggggccccgcgccgctgctgctggtcgggctggcgctgctgggcgcggcgcgggcgcgggaggaggcgggcggcggcttcagcctgcacccgccctacttcaacctggccgagggcgcccgcatcgccgcctccgcgacctgcggagaggaggccccggcgcgcggctccccgcgccccaccgaggacctttactgcaagctggtagggggccccgtggccggcggcgaccccaaccagaccatccggggccagtactgtgacatctgcacggctgccaacagcaacaaggcacaccccgcgagcaatgccatcgatggcacggagcgctggtggcagagtccaccgctgtcccgcggcctggagtacaacgaggtcaacgtcaccctggacctgggccaggtcttccacgtggcctacgtcctcatcaagtttgccaactcaccccggccggacctctgggtgctggagcggtccatggacttcggccgcacctaccagccctggcagttctttgcctcctccaagagggactgtctggagcggttcgggccacagacgctggagcgcatcacacgggacgacgcggccatctgcaccaccgagtactcacgcatcgtgcccctggagaacggagagatcgtggtgtccctggtgaacggacgtccgggcgccatgaatttctcctactcgccgctgctacgtgagttcaccaaggccaccaacgtccgcctgcgcttcctgcgtaccaacacgctgctgggccatctcatggggaaggcgctgcgggaccccacggtcacccgccggtattattacagcatcaaggatatcagcatcggaggccgctgtgtctgccacggccacgcggatgcctgcgatgccaaagaccccacggacccgttcaggctgcagtgcacctgccagcacaacacctgcgggggcacctgcgaccgctgctgccccggcttcaatcagcagccgtggaagcctgcgactgccaacagtgccaacgagtgccagtcctgtaactgctacggccatgccaccgactgttactacgaccctgaggtggaccggcgccgcgccagccagagcctggatggcacctatcagggtgggggtgtctgtatcgactgccagcaccacaccaccggcgtcaactgtgagcgctgcctgcccggcttctaccgctctcccaaccaccctctcgactcgccccacgtctgccgccgctgcaactgcgagtccgacttcacggatggcacctgcgaggacctgacgggtcgatgctactgccggcccaacttctctggggagcggtgtgacgtgtgtgccgagggcttcacgggcttcccaagctgctacccgacgccctcgtcctccaatgacaccagggagcaggtgctgccagccggccagattgtgaattgtgactgcagcgcggcagggacccagggcaacgcctgccggaaggacccaagggtgggacgctgtctgtgcaaacccaacttccaaggcacccattgtgagctctgcgcgccagggttctacggccccggctgccagccctgccagtgttccagccctggagtggccgatgaccgctgtgaccctgacacaggccagtgcaggtgccgagtgggcttcgagggggccacatgtgatcgctgtgcccccggctactttcacttccctctctgccagttgtgtggctgcagccctgcaggaaccttgcccgagggctgcgatgaggccggccgctgcctatgccagcctgagtttgctggacctcattgtgaccggtgccgccctggctaccatggtttccccaactgccaagcatgcacctgcgaccctcggggagccctggaccagctctgtggggcgggaggtttgtgccgctgccgccccggctacacaggcactgcctgccaggaatgcagccccggctttcacggcttccccagctgtgtcccctgccactgctctgctgaaggctccctgcacgcagcctgtgacccccggagtgggcagtgcagctgccggccccgtgtgacggggctgcggtgtgacacatgtgtgcccggtgcctacaacttcccctactgcgaagctggctcttgccaccctgccggtctggccccagtggatcctgcccttcctgaggcacaggttccctgtatgtgccgggctcacgtggaggggccgagctgtgaccgctgcaaacctgggttctggggactgagccccagcaaccccgagggctgtacccgctgcagctgcgacctcaggggcacactgggtggagttgctgagtgccagccgggcaccggccagtgcttctgcaagccccacgtgtgcggccaggcctgcgcgtcctgcaaggatggcttctttggactggatcaggctgactattttggctgccgcagctgccggtgtgacattggcggtgcactgggccagagctgtgaaccgaggacgggcgtctgccggtgccgccccaacacccagggccccacctgcagcgagcctgcgagggaccactacctcccggacctgcaccacctgcgcctggagctggaggaggctgccacacctgagggtcacgccgtgcgctttggcttcaaccccctcgagttcgagaacttcagctggaggggctacgcgcagatggcacctgtccagcccaggatcgtggccaggctgaacctgacctcccctgaccttttctggctcgtcttccgatacgtcaaccggggggccatgagtgtgagcgggcgggtctctgtgcgagaggagggcaggtcggccacctgcgccaactgcacagcacagagtcagcccgtggccttcccacccagcacggagcctgccttcatcaccgtgccccagaggggcttcggagagccctttgtgctgaaccctggcacctgggccctgcgtgtggaggccgaaggggtgctcctggactacgtggttctgctgcctagcgcatactacgaggcggcgctcctgcagctgcgggtgactgaggcctgcacataccgtccctctgcccagcagtctggcgacaactgcctcctctacacacacctccccctggatggcttcccctcggccgccgggctggaggccctgtgtcgccaggacaacagcctgccccggccctgccccacggagcagctcagcccgtcgcacccgccactgatcacctgcacgggcagtgatgtggacgtccagcttcaagtggcagtgccacagccaggccgctatgccctagtggtggagtacgccaatgaggatgcccgccaggaggtgggcgtggccgtgcacaccccacagcgggccccccagcaggggctgctctccctgcacccctgcctgtacagcaccctgtgccggggcactgcccgggatacccaggaccacctggctgtcttccacctggactcggaggccagcgtgaggctcacagccgaacaggcacgcttcttcctgcacggggtcactctggtgcccattgaggagttcagcccggagttcgtggagccccgggtcagctgcatcagcagccacggcgcctttggccccaacagtgccgcctgtctgccctcgcgcttcccaaagccgccccagcccatcatcctcagggactgccaggtgatcccgctgccgcccggcctcccgctgacccacgcgcaggatctcactccagccatgtccccagctggaccccgacctcggccccccaccgctgtggaccctgatgcagagcccaccctgctgcgtgagccccaggccaccgtggtcttcaccacccatgtgcccacgctgggccgctatgccttcctgctgcacggctaccagccagcccaccccaccttccccgtggaagtcctcatcaacgccggccgcgtgtggcagggccacgccaacgccagcttctgtccacatggctacggctgccgcaccctggtggtgtgtgagggccaggccctgctggacgtgacccacagcgagctcactgtgaccgtgcgtgtgcccaagggccggtggctctggctggattatgtactcgtggtccctgagaacgtctacagctttggctacctccgggaggagcccctggataaatcctatgacttcatcagccactgcgcagcccagggctaccacatcagccccagcagctcatccctgttctgccgaaacgctgctgcttccctctccctcttctataacaacggagcccgtccatgtggctgccacgaagtaggtgctacaggccccacgtgtgagcccttcgggggccagtgtccctgccatgcccatgtcattggccgtgactgctcccgctgtgccaccggatactggggcttccccaactgcaggccctgtgactgcggtgcccgcctctgtgacgagctcacgggccagtgcatctgcccgccacgcaccatcccgcccgactgcctgctgtgccagccccagacctttggctgccaccccctggtcggctgtgaggagtgtaactgctcagggcccggcatccaggagctcacagaccctacctgtgacacagacagcggccagtgcaagtgcagacccaacgtgactgggcgccgctgtgatacctgctctccgggcttccatggctacccccgctgccgcccctgtgactgtcacgaggcgggcactgcgcctggcgtgtgtgaccccctcacagggcagtgctactgtaaggagaacgtgcagggccccaaatgtgaccagtgcagccttgggaccttctcactggatgctgccaaccccaaaggttgcacccgctgcttctgctttggggccacggagcgctgccggagctcgtcctacacccgccaggagttcgtggatatggagggatgggtgctgctgagcactgaccggcaggtggtgccccacgagcggcagccagggacggagatgctccgtgcagacctgcggcacgtgcctgaggctgtgcccgaggctttccccgagctgtactggcaggccccaccctcctacctgggggaccgggtgtcatcctacggtgggaccctccgttatgaactgcactcagagacccagcggggagatgtctttgtccccatggagagcaggccggatgtggtgctgcagggcaaccagatgagcatcacattcctggagccggcataccccacgcctggccacgttcaccgtgggcagctgcagctggtggaggggaacttccggcatacggagacgcgcaacactgtgtcccgcgaggagctcatgatggtgctggccagcctggagcagctgcagatccgtgccctcttctcacagatctcctcggctgtcttcctgcgcagggtggcactggaggtggccagcccagcaggccagggggccctggccagcaatgtggagctgtgcctgtgccccgccagctaccggggggactcatgccaggaatgtgcccccggcttctatcgggacgtcaaaggtctcttcctgggccgatgtgtcccttgtcagtgccatggacactcagaccgctgcctccctggctctggcgtctgtgtggactgccagcacaacaccgaaggggcccactgtgagcgctgccaggctggcttcgtgagcagcagggacgaccccagcgccccctgtgtcagctgcccctgccccctctcagtgccttccaacaacttcgccgagggctgtgtcctgcgaggcggccgcacccagtgcctctgcaaacctggttatgcaggtgcctcctgcgagcggtgtgcgcccggattctttgggaacccactggtgctgggcagctcctgccagccatgcgactgcagcggcaacggtgaccccaacttgctcttcagcgactgcgaccccctgacgggcgcctgccgtggctgcctgcgccacaccactgggccccgctgcgagatctgtgcccccggcttctacggcaacgccctgctgcccggcaactgcacccggtgcgactgtaccccatgtgggacagaggcctgcgacccccacagcgggcactgcctgtgcaaggcgggcgtgactgggcggcgctgtgaccgctgccaggagggacattttggtttcgatggctgcgggggctgccgcccgtgtgcttgtggaccggccgccgagggctccgagtgccacccccagagcggacagtgccactgccgaccagggaccatgggaccccagtgccgcgagtgtgcccctggctactgggggctccctgagcagggctgcaggcgctgccagtgccctgggggccgctgtgaccctcacacgggccgctgcaactgccccccggggctcagcggggagcgctgcgacacctgcagccagcagcatcaggtgcctgttccaggcgggcctgtgggccacagcatccactgtgaagtgtgtgaccactgtgtggtcctgctcctggatgacctggaacgggccggcgccctcctccccgccattcacgagcaactgcgtggcatcaatgccagctccatggcctgggcccgtctgcacaggctgaacgcctccatcgctgacctgcagagccagctccggagccccctgggcccccgccatgagacggcacagcagctggaggtgctggagcagcagagcacaagcctcgggcaggacgcacggcggctaggcggccaggcagccgtggggacccgagaccaggcgagccaattgctggccggcaccgaggccacactgggccatgcgaagacgctgttggcggccatccgggctgtggaccgcaccctgagcgagctcatgtcccagacgggccacctggggctggccaatgcctcggctccatcaggtgagcagctgctccggacactggccgaggtggagcggctgctctgggagatgcgggcccgggacctgggggccccgcaggcagcagctgaggctgagttggctgcagcacagagattgctggcccgggtgcaggagcagctgagcagcctctgggaggagaaccaggcactggccacacaaacccgcgaccggctggcccagcacgaggccggcctcatggacctgcgagaggctttgaaccgggcagtggacgccacacgggaggcccaggagctcaacagccgcaaccaggagcgcctggaggaagccctgcaaaggaagcaggagctgtcccgggacaatgccaccctgcaggccactctgcatgcggctagggacaccctggccagcgtcttcagattgctgcacagcctggaccaggctaaggaggagctggagcgcctcgccgccagcctggatggggctcggaccccactgctgcagaggatgcagaccttctccccggcgggcagcaagctgcgtctagtggaggccgccgaggcccacgcacagcagctgggccagctggcactcaatctgtccagcatcatcctggacgtcaaccaggaccgcctcacccagagggccatcgaggcctccaacgcctacagccgcatcctgcaggccgtgcaggctgccgaggatgctgctggccaggccctgcagcaggcggaccacacgtgggcgacggtggtgcggcagggcctggtggaccgagcccagcagctcctggccaacagcactgcactagaagaggccatgctccaggaacagcagaggctgggccttgtgtgggctgccctccagggtgccaggacccagctccgagatgtccgggccaagaaggaccagctggaggcgcacatccaggcggcgcaggccatgcttgccatggacacagacgagacaagcaagaagatcgcacatgccaaggctgtggctgctgaagcccaggacaccgccacccgtgtgcagtcccagctgcaggccatgcaggagaatgtggagcggtggcagggccagtacgagggcctgcggggccaggacctgggccaggcagtgcttgacgcaggccactcagtgtccaccctggagaagacgctgccccagctgctggccaagctgagcatcctggagaaccgtggggtgcacaacgccagcctggccctgtccgccagcattggccgcgtgcgagagctcattgcccaggcccggggggctgccagtaaggtcaaggtgcccatgaagttcaacgggcgctcaggggtgcagctgcgcaccccacgggatcttgccgaccttgctgcctacactgccctcaagttctacctgcagggcccagagcctgagcctgggcagggtaccgaggatcgctttgtgatgtacatgggcagccgccaggccactggggactacatgggtgtgtctctgcgtgacaagaaggtgcactgggtgtatcagctgggtgaggcgggccctgcagtcctaagcatcgatgaggacattggggagcagttcgcagctgtcagcctggacaggactctccagtttggccacatgtccgtcacagtggagagacagatgatccaggaaaccaagggtgacacggtggcccctggggcagaggggctgctcaacctgcggccagacgacttcgtcttctacgtcggggggtaccccagtaccttcacgccccctcccctgcttcgcttccccggctaccggggctgcatcgagatggacacgctgaatgaggaggtggtcagcctctacaacttcgagaggaccttccagctggacacggctgtggacaggccttgtgcccgctccaagtcgaccggggacccgtggctcacggacggctcctacctggacggcaccggcttcgcccgcatcagcttcgacagtcagatcagcaccaccaagcgcttcgagcaggagctgcggctcgtgtcctacagcggggtgctcttcttcctgaagcagcagagccagttcctgtgcttggccgtgcaagaaggcagcctcgtgctgttgtatgactttggggctggcctgaaaaaggccgtcccactgcagcccccaccgcccctgacctcggccagcaaggcgatccaggtgttcctgctggggggcagccgcaagcgtgtgctggtgcgtgtggagcgggccacggtgtacagcgtggagcaggacaatgatctggagctggccgacgcctactacctggggggcgtgccgcccgaccagctgcccccgagcctgcgacggctcttccccaccggaggctcagtccgtggctgcgtcaaaggcatcaaggccctgggcaagtatgtggacctcaagcggctgaacacgacaggcgtgagcgccggctgcaccgccgacctgctggtggggcgcgccatgactttccatggccacggcttccttcgcctggcgctctcgaacgtggcaccgctcactggcaacgtctactccggcttcggcttccacagcgcccaggacagtgccctgctctactaccgggcgtccccggatgggctatgccaggtgtccctgcagcagggccgtgtgagcctacagctcctgaggactgaagtgaaaactcaagcgggcttcgccgatggtgccccccattacgtcgccttctacagcaatgccacgggagtctggctgtatgtcgatgaccagctccagcagatgaagccccaccggggaccaccccccgagctccagccgcagcctgaggggcccccgaggctcctcctgggaggcctgcctgagtctggcaccatttacaacttcagtggctgcatcagcaacgtcttcgtgcagcggctcctgggcccacagcgcgtatttgatctgcagcagaacctgggcagcgtcaatgtgagcacgggctgtgcacccgccctgcaagcccagaccccgggcctggggcctagaggactgcaggccaccgcccggaaggcctcccgccgcagccgtcagcccgcccggcatcctgcctgcatgctgcccccacacctcaggaccacccgagactcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgcatcttgggccccctggaggcgggcctgttcttcccaggcagcgggggagttatcactttagacctcccaggagctacactgcctgatgtgggcctggaactggaggtgcggcccctggcagtcaccggactgatcttccacttgggccaggcccggacgcccccctacttgcagttgcaggtgaccgagaagcaagtcctgctgcgggcggatgacggagcaggggagttctccacgtcagtgacccgcccctcagtgctgtgtgatggccagtggcaccggctagcggtgatgaaaagcgggaatgtgctccggctggaggtggacgcgcagagcaaccacaccgtgggccccttgctggcggctgcagctggtgccccagcccctctgtacctcgggggcctgcctgagcccatggccgtgcagccctggccccccgcctactgcggctgcatgaggaggctggcggtgaaccggtcccccgtcgccatgactcgctctgtggaggtccacggggcagtgggggccagtggctgcccagccgcctag

SEQ ID NO: 10 - DNA, 1014 bp, Homo sapiens
atgacaaacaacagcggctccaaagccgaactcgttgtgggagggaaatacaaactggtgcggaagatcgggtctggctcctttggagacgtttatctgggcatcaccaccaccaacggcgaggacgtagcagtgaagctggaatctcagaaggtcaagcacccccagttgctgtatgagagcaaactctacacgattcttcaaggtggggttggcatcccccacatgcactggtatggtcaggaaaaagacaacaatgtgctagtcatggaccttctgggacccagcctcgaagacctctttaatttctgttcaagaaggttcaccatgaaaactgtacttatgttagccgaccagatgatcagcagaattgaatacgtgcatacaaagaattttctacaccgagacattaaaccagataacttcctgatgggtactgggcgtcactgtaataagttgttccttattgattttggtttggccaaaaagtacagagacaacaggaccaggcaacacataccgtacagagaagataaacacctcattggcactgtccgatatgccagcatcaatgcacatcttggtattgagcagagccgccgagatgacatggaatccttaggctacgttttcatgtattttaatagaaccagcctgccgtggcaaggactaagggctatgacaaaaaaacaaaaatatgaaaagattagtgagaagaagatgtccacccctgttgaagttttatgtaaggggtttcctgcagaattcgccatgtacttgaactactgtcgtgggctgcgctttgaggaagtcccagattacatgtatctgaggcagctattccgcattcttttcaggaccctgaaccaccaatatgactacacatttgattggacgatgttaaagcagaaagcagcacagcaggcagcctcttccagtgggcagggtcagcaggcccaaacccagacaggcaagcaaactgaaaaaaacaagaataatgtgaaagataactaa

SEQ ID NO: 11 - DNA, 2667 bp, Homo sapiens
atggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacagatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcagtaattgtttaaccatcacttgtctcctcggctgttttataaaaggacaaaacagcaagtgtctgcattgcatctcggtttctcccattagcgaggagcaagacaaaaagacagaggagaaagtgagcctaaggggaaagagaattgctgagtactcctatgggcatcaaaagaaatcaagcaagaagaaaaaaatcagtaagaatgacatccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgaggtgagtgcagctaagtggctctgtgaggttctcccaggtctccttctttag

SEQ ID NO: 12 - DNA, 2568 bp, Homo sapiens
atggagtcgctcctgctgccggtgctgctgctgctggccatactgtggacgcaggctgccgccctcattaatctcaagtactcggtagaagaggagcagcgcgccgggacggtgattgccaacgtggccaaagacgcgcgagaggcgggcttcgcgctggacccccggcaggcttcagcctttcgcgtggtgtccaactcggctccacacctagtggacatcaatcccagctctggcctgctggtcaccaagcagaagattgaccgtgatctgctgtgccgccagagccccaagtgcatcatctcgctcgaggtcatgtccagctcaatggaaatctgcgtgataaaggtggagatcaaggacctgaacgacaatgcgcccagtttcccggcagcacagatcgagctggagatctcggaggcagccagccctggcacgcgcatcccgctggacagcgcttacgatccagactcaggaagctttggcgtgcagacttacgagctcacgcccaacgagctgttcggcctggagatcaagacgcgcggcgacggctcccgctttgccgaactcgtggtggaaaagagcctggaccgcgagacgcagtcgcactacagcttccgaatcactgcgctagacggtggcgacccgccgcgcctgggcaccgttggccttagtatcaaggtgaccgactccaatgacaacaacccggtgtttagcgagtccacctacgcggtgagcgtgccagaaaactcgcctcccaacacacccgtcatccgcctcaacgccagcgatccagacgagggcaccaacggccaggtggtctactccttctatggctacgtcaacgaccgcacgcgcgagctctttcagatcgacccgcacagtggcctggtcactgtcactggcgctttagactacgaagaggggcacgtgtacgaactggacgtgcaggctaaggacttggggcccaattccatcccggcacactgcaaggtcaccgtcagcgtgctggacaccaatgacaatccgccggtcatcaacctgctgtcagtcaacagtgagcttgtggaggtcagcgagagcgcccccccgggctacgtgatcgccttggtgcgggtgtctgatcgcgactcaggcctcaatggacgtgtgcagtgccgtttgctgggcaatgtgccctttcgactgcaggaatatgagagcttctccactattctggtggacggacggctggaccgcgagcagcacgaccaatacaacctcacaattcaggcacgcgacggcggcgtgcccatgctgcagagtgccaagtcctttaccgtgctcatcactgacgaaaatgacaaccacccgcacttttccaagccctactaccaggtcattgtgcaggagaacaacacgcctggcgcctatctgctctctgtgtctgctcgcgaccccgacctgggtctcaacggcagtgtctcctaccagatcgtgccgtcgcaggtgcgggacatgcctgtcttcacctatgtctccatcaatcccaactcaggcgacatctacgcgctgcgatcctttaaccacgagcagaccaaggcgttcgaattcaaggtgctggccaaggacggcggccttccctcactgcaaagcaacgctacggtgcgggtcatcatcctcgacgtcaacgacaacaccccggtcatcacagccccacctctgattaacggcactgccgaggtctacataccccgcaactctggcataggctacctggtgactgttgtcaaggcagaagactacgatgagggcgaaaatggccgagtcacctacgacatgaccgagggcgaccgcggcttctttgaaatagaccaggtcaatggcgaagtcagaaccacccgcaccttcggggagagctccaagtcctcctatgagcttatcgtggtggctcacgaccacggcaagacatctctctctgcctctgctctcgtcctaatctacttgtcccctgctctcgatgcccaagagtcaatgggctctgtgaacttgtccttgattttcattattgccctgggctccattgcgggcatcctctttgtaactatgatcttcgtggcaatcaagtgcaagcgagacaacaaagagatccggacctacaactgcagaattgctgagtactcctatgggcatcaaaagaaatcaagcaagaagaaaaaaatcagtaagaatgacatccgcctggtaccccgggatgtggaggagacagacaagatgaacgttgtcagttgctcttccctgacctcctccctcaactattttgactaccaccagcagacgctgcccctgggctgccgccgctctgagagcactttcctgaatgtggagaaccagaatacccgcaacaccagtgctaaccacatctaccatcactctttcaacagccaggggccccagcagcctgacctgattatcaacggtgtgcctctgcctgagactgaaaactattcttttgactccaactacgtgaatagccgagcccatttaatcaagaggtatgttggtttgcttgcttattgctgcaactaa

SEQ ID NO: 13 - DNA, 990 bp, Homo sapiens
atggtgacgaaggcctttgtcttgttggccatctttgcagaagcctctgcaaaatcgtgtgctccaaataaagcagatgtcattcttgtgttttgctatcccaaaaccatcatcaccaaaatccccgagtgtccctatggatgggaagttcatcagctggccctcggagggctgtgttacaatggggtccacgaaggaggttactaccaatttgtgatcccagatttatcacctaaaaacaagtcctattgtggaacccagtctgagtacaagccacctatctatcacttctacagtcacatcgtttccaatgacaccacagtgattgtaaaaaaccagcctgtcaactactccttctcctgcacctaccactccacctacttggtgaaccaggctgcctttgaccagagagtggccactgttcacgtgaagaacgggagcatgggcacatttgagagccaactgtctctcaacttctacactaatgccaagttctccatcaagaaagaagctccctttgtcctggaggcatccgaaatcggttcagatctgtttgcaggagtggaagccaaagggttaagcattaggtttaaagtggtcttgaacagctgttgggccaccccctcggctgacttcatgtatcccttgcagtggcagctgatcaacaagggctgccccacggatgaaaccgtcctcgtgcatgagaatgggagagatcacagggcaaccttccaattcaatgctttccggttccagaacatccccaaactctccaaggtgtggttacactgtgagacgttcatctgcgacagtgagaaactctcctgcccagtgacctgcgataaacggaagcgcctcctgcgagaccagaccgggggagtcctggtcgtggagctctccctgcggagcaggggattttccagtctctatagcttctcagatgttctccaccacctcatcatgatgttggggatttgtgccgtgttatag

SEQ ID NO: 14 - DNA, 699 bp, Homo sapiens
atgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacaggcctcgaccctgcaggccctttattcaacgggaaacctcaccaagacagattagatcccagtgatgcgcagtttgttgatgtcatccattccgacactgatggtaacgctcctttccttgtggcactgggctacaaggagccattaggaaacatagacttctacccaaatggaggattggatcaacctggctgccccaaaacaatattgggaggaaatgttaaggaaatgatacaggcttcctatatctttttccttaaaaacgactctatggacttaagttcaccgaaggaagtggaatga

SEQ ID NO: 15 - DNA, 1359 bp, Homo sapiens
atgttgagattctacttattcatcagtttgttgtgcttgtcaagatcagacgcagaagaaacatgtccttcattcaccaggctgagctttcacagtgcagtggttggtacgggactaaatgtgaggctgatgctctacacaaggaaaaacctgacctgcgcacaaaccatcaactcctcagcttttgggaacttgaatgtgaccaagaaaaccaccttcattgtccatggattcaggccaacaggctcccctcctgtttggatggatgacttagtaaagggtttgctctctgttgaagacatgaacgtagttgttgttgattggaatcgaggagctacaactttaatatatacccatgcctctagtaagaccagaaaagtagccatggtcttgaaggaatttattgaccagatgttggcagaaggagcttctcttgatgacatttacatgatcggagtaagtctaggagcccacatatctgggtttgttggagagatgtacgatggatggctggggagaattacaggcctcgaccctgcaggccctttattcaacgggaaacctcaccaagacagattagatcccagtgatgcgcagtttgttgatgtcatccattccgacactgatgcactgggctacaaggagccattaggaaacatagacttctacccaaatggaggattggatcaacctggctgccccaaaacaatattgggaggatttcagtattttaaatgtgaccaccagaggtctgtatacctgtacctgtcttccctgagagagagctgcaccatcactgcgtatccctgtgactcctaccaggattataggaatggcaagtgtgtcagctgcggcacgtcacaaaaagagtcctgtccccttctgggctattatgctgataattggaaagaccatctaagggggaaagatcctccaatgacgaaggcattctttgacacagctgaggagagcccattctgcatgtatcattactttgtggatattataacatgggacaagaatgtaagaagaggggacattaccatcaaattgagagacaaagctggaaacacccacagatccaaaatcatcagtaatgaacccaccacatttcagaaatatcaccaagtgagtctacttgcaagatttaatcaagatctggataaagtggctgcaatttccttgatgttctctacaggatctctaataggcccaaggtacaagctcaggattctccgaatgaagttaaggtcccttgcccatccggagaggcctcagctgtgtcggtatgatcttgtcctgatggaaaacgttgaaacagtcttccaacctattctttgcccagagttgcagttgtaa

SEQ ID NO: 16 - DNA, 1353 bp, Homo sapiens
atggggctccggagccaccacctcagcctgggccttctgcttctgtttctactccctgcagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacggaaccaggtgctgaccctgtatctgtggatacggcaggagtggacagatgcctacctacgatgggaccccaatgcctatggtggcctggatgccatccgcatccccagcagtcttgtgtggcggccagacatcgtactctataacaaagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctgcctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcatgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctgtga

SEQ ID NO: 17 - DNA, 768 bp, Homo sapiens
atggttaagggtgagaaaggccccaagggcaagaagatcaccctcaaggtggccaggaattgcatcaaaatcacttttgatgggaaaaagcgccttgacttgagcaagatgggaattaccaccttccccaagtgtattctgcgccttagtgacatggacgagctggaccttagccggaatcttatcaggaagatccctgactccatctccaagttccagaacctccggtggctggacctgcacagcaactacatagacaagctgcctgagtccattggccagatgaccagcctgctctacctcaacgtcagcaacaaccggctgaccagcaacgggctgcccgtggagctgaagcaactcaagaacatccgcgctgtgaacctaggcttgaaccacctggacagcgtgcccaccacactgggggccctgaaggagctccacgaggtagggctccatgacaacctactgaacaacatccccgtgagcatctccaagctccccaagctgaaaaagctcaacataaagcggaacccctttccaaagccaggtgagtcggaaatattcatagactccatcaggaggctggagaacttgtatgttgtggaggagaaggatctgtgtgcggcttgcctgagaaaatgccaaaacgcccgggacaacctgaatagaatcaagaacatggccacgacgacaccgagaaagaccatctttcccaatctgatctcacccaattccatggccaaggactcctgggaagactggaggtga

SEQ ID NO: 18 - DNA, 645 bp, Homo sapiens
atgcaggcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatga

SEQ ID NO: 19 - DNA, 696 bp, Homo sapiens
atgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatga

SEQ ID NO: 20 - DNA, 792 bp, Homo sapiens
atgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatcatgttctcatgcagcatgaaaagctctcaccagaagccaagtggatgctggcagcacatttcttgtaacttcccaggctgcagaacattggctaaatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatga

SEQ ID NO: 21 - DNA, 780 bp, Homo sapiens
atgtatgtattatctccagtggaatttataattctacaacttttatttattcaggccatttccagcagtttaaaaggtttcctttcagctatgagactggctcatagaggctgtaatgttgatacaccagtttcaacgctcacaccagtgaagacttcagaatttgaaaactttaaaactaaaatggttatcacatccaaaaaagactatcctctaagtaagaattttccatattccttggaacatcttcagacttcttactgtgggcttgtccgagttgatatgcgtatgctttgcttaaaaagccttaggaaattagacttgagtcacaaccatataaaaaagcttccagctacaattggagacctcatacaccttcaagaacttaacctgaatgacaatcacttggagtcatttagtgtagccttgtgtcattctacactccagaagtcacttcggagtttggacctcagcaagaacaaaatcaaggcactccctgtgcagttttgccagctccaggaacttaagaatttaaaacttgacgataatgaattgattcaatttccttgcaagataggacaactaataaaccttcgctttttgtcagcagctcgaaataagcttccatttttgcctagtgaatttagaaatttatcccttgaatacttggatctttttggaaatacttttgaacaaccaaaagtccttccagtaataaagctgcaagcaccattaactttattggaatcttctgcacgaaccatattacataataggtaa

SEQ ID NO: 22 - DNA, 1251 bp, Homo sapiens
atgaagctacactgtgaggtggaggtgatcagccggcacttgcccgccttggggcttaggaaccggggcaagggcgtccgagccgtgttgagcctctgtcagcagacttccaggagtcagccgccggtccgagccttcctgctcatctccaccctgaaggacaagcgcgggacccgctatgagctaagggagaacattgagcaattcttcaccaaatttgtagatgaggggaaagccactgttcggttaaaggagcctcctgtggatatctgtctaagtaaggccatttccagcagtttaaaaggtttcctttcagctatgagactggctcatagaggctgtaatgttgatacaccagtttcaacgctcacaccagtgaagacttcagaatttgaaaactttaaaactaaaatggttatcacatccaaaaaagactatcctctaagtaagaattttccatattccttggaacatcttcagacttcttactgtgggcttgtccgagttgatatgcgtatgctttgcttaaaaagccttaggaaattagacttgagtcacaaccatataaaaaagcttccagctacaattggagacctcatacaccttcaagaacttaacctgaatgacaatcacttggagtcatttagtgtagccttgtgtcattctacactccagaagtcacttcggagtttggacctcagcaagaacaaaatcaaggcactccctgtgcagttttgccagctccaggaacttaagaatttaaaacttgacgataatgaattgattcaatttccttgcaagataggacaactaataaaccttcgctttttgtcagcagctcgaaataagcttccatttttgcctagtgaatttagaaatttatcccttgaatacttggatctttttggaaatacttttgaacaaccaaaagtccttccagtaataaagctgcaagcaccattaactttattggaatcttctgcacgaaccatattacataataggaataggattccatatggctctcatatcattccattccatctctgccaagatttggataccgcaaaaatttgtgtttgtggaagattctgtctgaactctttcattcaaggaactactaccatgaatctgcattctgttgcccacactgtggtcttagtagataatttgggtggtactgaagcacctattatctcttatttctgttctctaggctgttatgttaattcctctgatatgttaaagtaa

SEQ ID NO: 23 - protein, 461 aa, Homo sapiens
MLGIWIVAFLFFGTSRGKEVCYERLGCFKDGLPWTRTFSTELVGLPWSPEKINTRFLLYTIHNPNAYQEISAVNSSTIQASYFGTDKITRINIAGWKTDGKWQRDMCNVLLQLEDINCINLDWINGSREYIHAVNNLRVVGAEVAYFIDVLMKKFEYSPSKVHLIGHSLGAHLAGEAGSRIPGLGRITGLDPAGPFFHNTPKEVRLDPSDANFVDVIHTNAARILFELGVGTIDACGHLDFYPNGGKHMPGCEDLITPLLKFNFNAYKKEMASFFDCNHARSYQFYAESILNPDAFIAYPCRSYTSFKAGTCVGCADLLHRIDKIGSHTSHVFLTLSLPFLLVSLYLGWRHKLSVKLSGSEVTQGTVFLRVGGAVRKTGEFAIVSGKLEPGMTYTKLIDADVNVGNITSVQFIWKKHLFEDSQNKLGAEMVINTSGKYGYKSTFCSQDIMGPNILQNLKPC

SEQ ID NO: 24 - protein, 308 aa, Homo sapiens [sequence omitted]

SEQ ID NO: 25 - protein, 457 aa, Homo sapiens
MDLSRPRWSLWRRVFLMASLLACGICQASGQIFITQTLGIKGYRTVVALDKVPEDVQEYSWYWGANDSAGNMIISHKPPSAQQPGPMYTGRERVNREGSLLIRPTALNDTGNYTVRVVAGNETQRATGWLEVLELGSNLGISVNASSLVENMDSVAADCLTNVTNITWYVNDVPTSSSDRMTISPDGKTLVILRVSRYDRTIQCMIESFPEIFQRSERISLTVAYGPDYVLLRSNPDDFNGIVTAEIGSQVEMECICYSFLDLKYHWIHNGSLLNFSDAKMNLSSLAWEQMGRYRCTVENPVTQLIMYMDVRIQAPHECPLPSGILPVVHRDFSISGSMVMFLIMLTVLGGVYICGVLIHALINHYSIRCPHCSGTRVGCWLGAGTQEPALPPEGKQSQKGRDKPGTRLSGIIWGRQISPQDLKLMGAREGLESAMVLNSCGVSSSNFPSLCVYKGY

SEQ ID NO: 26 - protein, 704 aa, Homo sapiens [sequence omitted]

SEQ ID NO: 27 - protein, 361 aa, Homo sapiens
MKPLRRPLPFICPSPPSPRLTCLPPLALSSLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 28 - protein, 365 aa, Homo sapiens
MWLLLTTTCLICGTLNAGGFLDLENEVNPEVWMNTSEIIIYNGYPSEEYEVTTEDGYILLVNRIPYGRTHARSTADAGYDVWMGNSRGNTWSRRHKTLSETDEKFWAFSFDEMAKYDLPGVIDFIVNKTGQEKLYFIGHSLGTTIGFVAFSTMPELAQRIKMNFALGPTISFKYPTGIFTRFFLLPNSIIKAVFGTKGFFLEDKKTKIASTKICNNKILWLICSEFMSLWAGSNKKNMNQSRMDVYMSHAPTGSSVHNILHIKQLYHSDEFRAYDWGNDADNMKHYNQSHPPIYDLTAMKVPTAIWAGGHDVLVTPQDVARILPQIKSLHYFKLLPDWNHFDFVWGLDAPQRMYSEIIALMKAYS

SEQ ID NO: 29 - protein, 397 aa, Homo sapiens
MWQLLAAACWMLLLGSMYGYDKKGNNANPEANMNISQIISYWGYPYEEYDVTTKDGYILGIYRIPHGRGCPGRTAPKPAVYLQHGLIASASNWICNLPNNSLAFLLADSGYDVWLGNSRGNTWSRKHLKLSPKSPEYWAFSLDEMAKYDLPATINFIIEKTGQKRLYYVGHSQGTTIAFIAFSTNPELAKKIKIFFALAPVVTVKYTQSPMKKLTTLSRRVVKVLFGDKMFHPHTLFDQFIATKVCNRKLFRRICSNFLFTLSGFDPQNLNMSRLDVYLSHNPAGTSVQNMLHWAQLYHSDEFRAYDWGNDADNMKHYNQSHPPIYDLTAMKVPTAIWAGGHDVLVTPQDVARILPQIKSLHYFKLLPDWNHFDFVWGLDAPQRMYSEIIALMKAYS

SEQ ID NO: 30 - protein, 3705 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAGAPRPPRAPGGFHLYQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 31 - protein, 3696 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 32 - protein, 337 aa, Homo sapiens
MTNNSGSKAELVVGGKYKLVRKIGSGSFGDVYLGITTTNGEDVAVKLESQKVKHPQLLYESKLYTILQGGVGIPHMHWYGQEKDNNVLVMDLLGPSLEDLFNFCSRRFTMKTVLMLADQMISRIEYVHTKNFLHRDIKPDNFLMGTGRHCNKLFLIDFGLAKKYRDNRTRQHIPYREDKHLIGTVRYASINAHLGIEQSRRDDMESLGYVFMYFNRTSLPWQGLRAMTKKQKYEKISEKKMSTPVEVLCKGFPAEFAMYLNYCRGLRFEEVPDYMYLRQLFRILFRTLNHQYDYTFDWTMLKQKAAQQAASSSGQGQQAQTQTGKQTEKNKNNVKDN

SEQ ID NO: 33 - protein, 888 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCSNCLTITCLLGCFIKGQNSKCLHCISVSPISEEQDKKTEEKVSLRGKRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPEVSAAKWLCEVLPGLLL

SEQ ID NO: 34 - protein, 855 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKRYVGLLAYCCN

SEQ ID NO: 35 - protein, 329 aa, Homo sapiens
MVTKAFVLLAIFAEASAKSCAPNKADVILVFCYPKTIITKIPECPYGWEVHQLALGGLCYNGVHEGGYYQFVIPDLSPKNKSYCGTQSEYKPPIYHFYSHIVSNDTTVIVKNQPVNYSFSCTYHSTYLVNQAAFDQRVATVHVKNGSMGTFESQLSLNFYTNAKFSIKKEAPFVLEASEIGSDLFAGVEAKGLSIRFKVVLNSCWATPSADFMYPLQWQLINKGCPTDETVLVHENGRDHRATFQFNAFRFQNIPKLSKVWLHCETFICDSEKLSCPVTCDKRKRLLRDQTGGVLVVELSLRSRGFSSLYSFSDVLHHLIMMLGICAVL

SEQ ID NO: 36 - protein, 232 aa, Homo sapiens
MLYTRKNLTCAQTINSSAFGNLNVTKKTTFIVHGFRPTGSPPVWMDDLVKGLLSVEDMNVVVVDWNRGATTLIYTHASSKTRKVAMVLKEFIDQMLAEGASLDDIYMIGVSLGAHISGFVGEMYDGWLGRITGLDPAGPLFNGKPHQDRLDPSDAQFVDVIHSDTDGNAPFLVALGYKEPLGNIDFYPNGGLDQPGCPKTILGGNVKEMIQASYIFFLKNDSMDLSSPKEVE

SEQ ID NO: 37 - protein, 452 aa, Homo sapiens [sequence omitted]

SEQ ID NO: 38 - protein, 450 aa, Homo sapiens
MGLRSHHLSLGLLLLFLLPAECLGAEGRLALKLFRDLFANYTSALRPVADTDQTLNVTLEVTLSQIIDMDERNQVLTLYLWIRQEWTDAYLRWDPNAYGGLDAIRIPSSLVWRPDIVLYNKADAQPPGSASTNVVLRHDGAVRWDAPAITRSSCRVDVAAFPFDAQHCGLTFGSWTHGGHQLDVRPRGAAASLADFVENVEWRVLGMPARRRVLTYGCCSEPYPDVTFTLLLRRRAAAYVCNLLLPCVLISLLAPLAFHLPADSGEKVSLGVTVLLALTVFQLLLAESMPPAESVPLIGKYYMATMTMVTFSTALTILIMNLHYCGPSVRPVPAWARALLLGHLARGLCVRERGEPCGQSRPPELSPSPQSPEGGAGPPAGPCHEPRCLCRQEALLHHVATIANTFRSHRAAQRCHEDWKRLARVMDRFFLAIFFSMALVMSLLVLVQAL

SEQ ID NO: 39 - protein, 255 aa, Homo sapiens
MVKGEKGPKGKKITLKVARNCIKITFDGKKRLDLSKMGITTFPKCILRLSDMDELDLSRNLIRKIPDSISKFQNLRWLDLHSNYIDKLPESIGQMTSLLYLNVSNNRLTSNGLPVELKQLKNIRAVNLGLNHLDSVPTTLGALKELHEVGLHDNLLNNIPVSISKLPKLKKLNIKRNPFPKPGESEIFIDSIRRLENLYVVEEKDLCAACLRKCQNARDNLNRIKNMATTTPRKTIFPNLISPNSMAKDSWEDWR

SEQ ID NO: 40 - protein, 214 aa, Homo sapiens
MQAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 41 - protein, 231 aa, Homo sapiens
MMPKHCFLGFLISFFLTGVAGTQSTHESLKPQRVQFQSRNFHNILQWQPGRALTGNSSVYFVQYKIYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 42 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 43 - protein, 259 aa, Homo sapiens [sequence omitted]

SEQ ID NO: 44 - protein, 416 aa, Homo sapiens
MKLHCEVEVISRHLPALGLRNRGKGVRAVLSLCQQTSRSQPPVRAFLLISTLKDKRGTRYELRENIEQFFTKFVDEGKATVRLKEPPVDICLSKAISSSLKGFLSAMRLAHRGCNVDTPVSTLTPVKTSEFENFKTKMVITSKKDYPLSKNFPYSLEHLQTSYCGLVRVDMRMLCLKSLRKLDLSHNHIKKLPATIGDLIHLQELNLNDNHLESFSVALCHSTLQKSLRSLDLSKNKIKALPVQFCQLQELKNLKLDDNELIQFPCKIGQLINLRFLSAARNKLPFLPSEFRNLSLEYLDLFGNTFEQPKVLPVIKLQAPLTLLESSARTILHNRNRIPYGSHIIPFHLCQDLDTAKICVCGRFCLNSFIQGTTTMNLHSVAHTVVLVDNLGGTEAPIISYFCSLGCYVNSSDMLK

(19) United States
(12) Patent Application Publication
(10) Pub. No.: US 2003/0219862 A1
(43) Pub. Date: Nov. 27, 2003

(54) Novel compounds

(76) Inventors: Pankaj Agarwal, King of Prussia, PA (US); John P Cogswell, Research Park Triangle, NC (US); Karen S Kabnick, Lafayette Hill, PA (US); Ying-Ta Lai, Upper Darby, PA (US); Shelley A Martensen, Research Triangle Park, NC (US); Paul R Murdock, Harlow Essex (GB); Safia K Rizvi, Philadelphia, PA (US); Randall F Smith, Lafayette Hill, PA (US); Jay C Strum, Research Triangle Park, NC (US); Zhaoying Xiang, Fort Lee, NJ (US); Qing Xie, Horsham, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/312,088

(22) PCT Filed: Jun. 22, 2001
(86) PCT No.: PCT/US01/19929

(51) Int. Cl. A61K 38/17; C07K 14/47; C12P 21/02; C12N 5/06; C07H 21/04

(52) U.S. Cl. 435/69.1; 435/183; 435/320.1; 435/325; 530/350; 536/23.5; 514/12

(57) ABSTRACT

Polypeptides and polynucleotides of the genes set forth in Table I and methods for producing such polypeptides by recombinant techniques are disclosed. Also disclosed are methods for utilizing polypeptides and polynucleotides of the genes set forth in Table I in diagnostic assays.

### FIELD OF INVENTION

This invention relates to newly identified polypeptides and polynucleotides encoding such polypeptides, to their use in diagnosis and in identifying compounds that may be agonists, antagonists that are potentially useful in therapy, and to production of such polypeptides and polynucleotides. The polynucleotides and polypeptides of the present invention also relate to proteins with signal sequences which allow them to be secreted extracellularly or membrane-associated (hereinafter often referred collectively as secreted proteins or secreted polypeptides).

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superseding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

Proteins and polypeptides that are naturally secreted into blood, lymph and other body fluids, or secreted into the cellular membrane are of primary interest for pharmaceutical research and development. The reason for this interest is the relative ease to target protein therapeutics into their place of action (body fluids or the cellular membrane). The natural pathway for protein secretion into extracellular space is the endoplasmic reticulum in eukaryotes and the inner membrane in prokaryotes (Palade, 1975, Science, 189, 347; Milstein, Brownlee, Harrison, and Mathews, 1972, Nature New Biol., 239, 117; Blobel, and Dobberstein, 1975, J. Cell. Biol., 67, 835). On the other hand, there is no known natural pathway for exporting a protein from the exterior of the cells into the cytosol (with the exception of pinocytosis, a mechanism of snake venom toxin intrusion into cells). Therefore targeting protein therapeutics into cells poses extreme difficulties.

The secreted and membrane-associated proteins include but are not limited to all peptide hormones and their receptors (including but not limited to insulin, growth hormones, chemokines, cytokines, neuropeptides, integrins, kallikreins, lamins, melanins, natriuretic hormones, neuropsin, neurotropins, pituitiary hormones, pleiotropins, prostaglandins, secretogranins, selecting, thromboglobulins, thymosins), the breast and colon cancer gene products, leptin, the obesity gene protein and its receptors, serum albumin, superoxide dismutase, spliceosome proteins, 7TM (transmembrane) proteins also called as G-protein coupled receptors, immunoglobulins, several families of serine proteinases (including but not limited to proteins of the blood coagulation cascade, digestive enzymes), deoxyribonuclease I, etc.

Therapeutics based on secreted or membrane-associated proteins approved by FDA or foreign agencies include but are not limited to insulin, glucagon, growth hormone, chorionic gonadotropin, follicle stimulating hormone, luteinizing hormone, calcitonin, adrenocorticotropic hormone (ACTH), vasopressin, interleukines, interferones, immunoglobulins, lactoferrin (diverse products marketed by several companies), tissue-type plasminogen activator (Alteplase by Genentech), hyaulorindase (Wydase by Wyeth-Ayerst), dornase alpha (Pulmozyme\ by Genentech), Chymodiactin (chymopapain by Knoll), alglucerase (Ceredase by Genzyme), streptokinase (Kabikinase by Pharmacia) (Streptase by Astra), etc. This indicates that secreted and membrane-associated proteins have an established, proven history as therapeutic targets. Clearly, there is a need for identification and characterization of further secreted and membrane-associated proteins which can play a role in preventing, ameliorating or correcting dysfunction or disease, including but not limited to diabetes, breast-, prostate-, colon cancer and other malignant tumors, hyper- and hypotension, obesity, bulimia, anorexia, growth abnormalities, asthma, manic depression, dementia, delirium, mental retardation, Huntington's disease, Tourette's syndrome, schizophrenia, growth, mental or sexual development disorders, and dysfunctions of the blood cascade system including those leading to stroke. The proteins of the present invention which include the signal sequences are also useful to further elucidate the mechanism of protein transport which at present is not entirely understood, and thus can be used as research tools.

### SUMMARY OF THE INVENTION

The present invention relates to particular polypeptides and polynucleotides of the genes set forth in Table I, including recombinant materials and methods for their production. Such polypeptides and polynucleotides are of interest in relation to methods of treatment of certain diseases, including, but not limited to, the diseases set forth in Tables III and V, hereinafter referred to as “diseases of the invention”. In a further aspect, the invention relates to methods for identifying agonists and antagonists (e.g., inhibitors) using the materials provided by the invention, and treating conditions associated with imbalance of polypeptides and/or polynucleotides of the genes set forth in Table I with the identified compounds. In still a further aspect, the invention relates to diagnostic assays for detecting diseases associated with inappropriate activity or levels the genes set forth in Table I. Another aspect of the invention concerns a polynucleotide comprising any of the nucleotide sequences set forth in the Sequence Listing and a polypeptide comprising a polypeptide encoded by the nucleotide sequence. In another aspect, the invention relates to a polypeptide comprising any of the polypeptide sequences set forth in the Sequence Listing and recombinant materials and methods for their production. Another aspect of the invention relates to methods for using such polypeptides and polynucleotides. Such uses include the treatment of diseases, abnormalities and disorders (hereinafter simply referred to as diseases) caused by abnormal expression, production, function and or metabolism of the genes of this invention, and such diseases are readily apparent by those skilled in the art from the homology to other proteins disclosed for each attached sequence. In still another aspect, the invention relates to methods to identify agonists and antagonists using the materials provided by the invention, and treating conditions associated with the imbalance with the identified compounds. Yet another aspect of the invention relates to diagnostic assays for detecting diseases associated with inappropriate activity or levels of the secreted proteins of the present invention.

### DESCRIPTION OF THE INVENTION

In a first aspect, the present invention relates to polypeptides the genes set forth in Table I. Such polypeptides include:

(a) an isolated polypeptide encoded by a polynucleotide comprising a sequence set forth in the Sequence Listing, herein when referring to polynucleotides or polypeptides of the Sequence Listing, a reference is also made to the Sequence Listing referred to in the Sequence Listing;

(b) an isolated polypeptide comprising a polypeptide sequence having at least 95%, 96%, 97%, 98%, or 99% identity to a polypeptide sequence set forth in the Sequence Listing;

(c) an isolated polypeptide comprising a polypeptide sequence set forth in the Sequence Listing;

(d) an isolated polypeptide having at least 95%, 96%, 97%, 98%, or 99% identity to a polypeptide sequence set forth in the Sequence Listing;

(e) a polypeptide sequence set forth in the Sequence Listing; and

(f) an isolated polypeptide having or comprising a polypeptide sequence that has an Identity Index of 0.95, 0.96, 0.97, 0.98, or 0.99 compared to a polypeptide sequence set forth in the Sequence Listing;

(g) fragments and variants of such polypeptides in (a) to (f).

Polypeptides of the present invention are believed to be members of the gene families set forth in Table II. They are therefore of therapeutic and diagnostic interest for the reasons set forth in Tables III and V. The biological properties of the polypeptides and polynucleotides of the genes set forth in Table I are hereinafter referred to as “the biological activity” of polypeptides and polynucleotides of the genes set forth in Table I. Preferably, a polypeptide of the present invention exhibits at least one biological activity of the genes set forth in Table I.

Polypeptides of the present invention also include variants of the aforementioned polypeptides, including all allelic forms and splice variants. Such polypeptides vary from the reference polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or I amino acids are inserted, substituted or deleted, in any combination.

Preferred fragments of polypeptides of the present invention include an isolated polypeptide comprising an amino acid sequence having at least 30, 50 or 100 contiguous amino acids from an amino acid sequence set forth in the Sequence Listing, or an isolated polypeptide comprising an amino acid sequence having at least 30, 50 or 100 contiguous amino acids truncated or deleted from an amino acid sequence set forth in the Sequence Listing. Preferred fragments are biologically active fragments that mediate the biological activity of polypeptides and polynucleotides of the genes set forth in Table I, including those with a similar activity or an improved activity, or with a decreased undesirable activity. Also preferred are those fragments that are antigenic or immunogenic in an animal, especially in a human.

Fragments of a polypeptide of the invention may be employed for producing the corresponding full-length polypeptide by peptide synthesis; therefore, these variants may be employed as intermediates for producing the full-length polypeptides of the invention. A polypeptide of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

In a further aspect, the present invention relates to polynucleotides of the genes set forth in Table I. Such polynucleotides include:

(a) an isolated polynucleotide comprising a polynucleotide sequence having at least 95%, 96%, 97%, 98%, or 99% identity to a polynucleotide sequence set forth in the Sequence Listing;

(b) an isolated polynucleotide comprising a polynucleotide set forth in the Sequence Listing;

(c) an isolated polynucleotide having at least 95%, 96%, 97%, 98%, or 99% identity to a polynucleotide set forth in the Sequence Listing;

(d) an isolated polynucleotide set forth in the Sequence Listing;

(e) an isolated polynucleotide comprising a polynucleotide sequence encoding a polypeptide sequence having at least 95%, 96%, 97%, 98%, or 99% identity to a polypeptide sequence set forth in the Sequence Listing;

(f) an isolated polynucleotide comprising a polynucleotide sequence encoding a polypeptide set forth in the Sequence Listing;

(g) an isolated polynucleotide having a polynucleotide sequence encoding a polypeptide sequence having at least 95%, 96%, 97%, 98%, or 99% identity to a polypeptide sequence set forth in the Sequence Listing;

(h) an isolated polynucleotide encoding a polypeptide set forth in the Sequence Listing;

(i) an isolated polynucleotide having or comprising a polynucleotide sequence that has an Identity Index of 0.95, 0.96, 0.97, 0.98, or 0.99 compared to a polynucleotide sequence set forth in the Sequence Listing;

(j) an isolated polynucleotide having or comprising a polynucleotide sequence encoding a polypeptide sequence that has an Identity Index of 0.95, 0.96, 0.97, 0.98, or 0.99 compared to a polypeptide sequence set forth in the Sequence Listing; and

polynucleotides that are fragments and variants of the above mentioned polynucleotides or that are complementary to above mentioned polynucleotides, over the entire length thereof.

Preferred fragments of polynucleotides of the present invention include an isolated polynucleotide comprising an nucleotide sequence having at least 15, 30, 50 or 100 contiguous nucleotides from a sequence set forth in the Sequence Listing, or an isolated polynucleotide comprising a sequence having at least 30, 50 or 100 contiguous nucleotides truncated or deleted from a sequence set forth in the Sequence Listing.

Preferred variants of polynucleotides of the present invention include splice variants, allelic variants, and polymorphisms, including polynucleotides having one or more single nucleotide polymorphisms (SNPs).

Polynucleotides of the present invention also include polynucleotides encoding polypeptide variants that comprise an amino acid sequence set forth in the Sequence Listing and in which several, for instance from 50 to 30, from 30 to 20, from 20 to 10, from 10 to 5, from 5 to 3, from 3 to 2, from 2 to 1 or 1 amino acid residues are substituted, deleted or added, in any combination.

In a further aspect, the present invention provides polynucleotides that are RNA transcripts of the DNA sequences of the present invention. Accordingly, there is provided an RNA polynucleotide that:

(a) comprises an RNA transcript of the DNA sequence encoding a polypeptide set forth in the Sequence Listing;

(b) is a RNA transcript of a DNA sequence encoding a polypeptide set forth in the Sequence Listing;

(c) comprises an RNA transcript of a DNA sequence set forth in the Sequence Listing; or

(d) is a RNA transcript of a DNA sequence set forth in the Sequence Listing; and RNA polynucleotides that are complementary thereto.

The polynucleotide sequences set forth in the Sequence Listing show homology with the polynucleotide sequences set forth in Table II. A polynucleotide sequence set forth in the Sequence Listing is a cDNA sequence that encodes a polypeptide set forth in the Sequence Listing. A polynucleotide sequence encoding a polypeptide set forth in the Sequence Listing may be identical to a polypeptide encoding a sequence set forth in the Sequence Listing or it may be a sequence other than a sequence set forth in the Sequence Listing, which, as a result of the redundancy (degeneracy) of the genetic code, also encodes a polypeptide set forth in the Sequence Listing. A polypeptide of a sequence set forth in the Sequence Listing is related to other proteins of the gene families set forth in Table II, having homology and/or structural similarity with the polypeptides set forth in Table II. Preferred polypeptides and polynucleotides of the present invention are expected to have, inter alia, similar biological functions/properties to their homologous polypeptides and polynucleotides. Furthermore, preferred polypeptides and polynucleotides of the present invention have at least one activity of the genes set forth in Table I.

Polynucleotides of the present invention may be obtained using standard cloning and screening techniques from a cDNA library derived from mRNA from the tissues set forth in Table IV (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro- protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. A polynucleotide may also contain non-coding 5′ and 3′ sequences, such as transcribed, non-translated sequences, splicing and polyadenylation signals, ribosome binding sites and sequences that stabilize mRNA.

Polynucleotides that are identical, or have sufficient identity to a polynucleotide sequence set forth in the Sequence Listing, may be used as hybridization probes for cDNA and genomic DNA or as primers for a nucleic acid amplification reaction (for instance, PCR). Such probes and primers may be used to isolate full-length cDNAs and genomic clones encoding polypeptides of the present invention and to isolate cDNA and genomic clones of other genes (including genes encoding paralogs from human sources and orthologs and paralogs from other species) that have a high sequence similarity to sequences set forth in the Sequence Listing, typically at least 95% identity. Preferred probes and primers will generally comprise at least 15 nucleotides, preferably, at least 30 nucleotides and may have at least 50, if not at least 100 nucleotides. Particularly preferred probes will have between 30 and 50 nucleotides. Particularly preferred primers will have between 20 and 25 nucleotides.

A polynucleotide encoding a polypeptide of the present invention, including homologs from other species, may be obtained by a process comprising the steps of screening a library under stringent hybridization conditions with a labeled probe having a sequence set forth in the Sequence Listing or a fragment thereof, preferably of at least 15 nucleotides; and isolating full-length cDNA and genomic clones containing the polynucleotide sequence set forth in the Sequence Listing. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 microgram/ml denatured, sheared salmon sperm DNA; followed by washing the filters in 0.1×SSC at about 65° C. Thus the present invention also includes isolated polynucleotides, preferably with a nucleotide sequence of at least 100, obtained by screening a library under stringent hybridization conditions with a labeled probe having the sequence set forth in the Sequence Listing or a fragment thereof, preferably of at least 15 nucleotides.

The skilled artisan will appreciate that, in many cases, an isolated cDNA sequence will be incomplete, in that the region coding for the polypeptide does not extend all the way through to the 5′ terminus. This is a consequence of reverse transcriptase, an enzyme with inherently low “processivity” (a measure of the ability of the enzyme to remain attached to the template during the polymerisation reaction), failing to complete a DNA copy of the mRNA template during first strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al., Proc Nat Acad Sci USA 85, 8998-9002, 1988). Recent modifications of the technique, exemplified by the Marathon (trade mark) technology (Clontech Laboratories Inc.) for example, have significantly simplified the search for longer cDNAs. In the Marathon (trade mark) technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Polynucleotides may be introduced into host cells by methods described in many standard laboratory manuals, such as Davis et al., Basic Methods in Molecular Biology (1986) and Sambrook et al.(ibid). Preferred methods of introducing polynucleotides into host cells include, for instance, calcium phosphate transfection, DEAE-dextran mediated transfection, transvection, micro-injection, cationic lipid-mediated transfection, electroporation, transduction, scrape loading, ballistic introduction or infection.

Representative examples of appropriate hosts include bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., (ibid). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during intracellular synthesis, isolation and/or purification.

Polynucleotides of the present invention may be used as diagnostic reagents, through detecting mutations in the associated gene. Detection of a mutated form of a gene is characterized by the polynucleotides set forth in the Sequence Listing in the cDNA or genomic sequence and which is associated with a dysfunction. Will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques well known in the art.

Nucleic acids for diagnosis may be obtained from a subject's cells, such as from blood, urine, saliva, tissue biopsy or autopsy material. The genomic DNA may be used directly for detection or it may be amplified enzymatically by using PCR, preferably RT-PCR, or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences of the genes set forth in Table I. Perfectly matched sequences can be distinguished from mismatched duplexes by RNase digestion or by differences in melting temperatures. DNA sequence difference may also be detected by alterations in the electrophoretic mobility of DNA fragments in gels, with or without denaturing agents, or by direct DNA sequencing (see, for instance, Myers et al., Science (1985) 230:1242). Sequence changes at specific locations may also be revealed by nuclease protection assays, such as RNase and S1 protection or the chemical cleavage method (see Cotton et al., Proc Natl Acad Sci USA (1985) 85: 4397-4401).

An array of oligonucleotides probes comprising polynucleotide sequences or fragments thereof of the genes set forth in Table I can be constructed to conduct efficient screening of e.g., genetic mutations. Such arrays are preferably high density arrays or grids. Array technology methods are well known and have general applicability and can be used to address a variety of questions in molecular genetics including gene expression, genetic linkage, and genetic variability, see, for example, M. Chee et al., Science, 274, 610-613 (1996) and other references cited therein.

Detection of abnormally decreased or increased levels of polypeptide or mRNA expression may also be used for diagnosing or determining susceptibility of a subject to a disease of the invention. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radio-immunoassays, competitive-binding assays, Western Blot analysis and ELISA assays.

Thus in another aspect, the present invention relates to a diagnostic kit comprising:

(a) a polynucleotide of the present invention, preferably the nucleotide sequence set forth in the Sequence Listing, or a fragment or an RNA transcript thereof;

(b) a nucleotide sequence complementary to that of (a);

(c) a polypeptide of the present invention, preferably the polypeptide set forth in the Sequence Listing or a fragment thereof; or

(d) an antibody to a polypeptide of the present invention, preferably to the polypeptide set forth in the Sequence Listing.

It will be appreciated that in any such kit, (a), (b), (c) or (d) may comprise a substantial component. Such a kit will be of use in diagnosing a disease or susceptibility to a disease, particularly diseases of the invention, amongst others.

The polynucleotide sequences of the present invention are valuable for chromosome localisation studies. The sequences set forth in the Sequence Listing are specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (co-inheritance of physically adjacent genes). Precise human chromosomal localisations for a genomic sequence (gene fragment etc.) can be determined using Radiation Hybrid (RH) Mapping (Walter, M. Spillett, D., Thomas, P., Weissenbach, J., and Goodfellow, P., (1994) A method for constructing radiation hybrid maps of whole genomes, Nature Genetics 7, 22-28). A number of RH panels are available from Research Genetics (Huntsville, Ala., USA) e.g. the GeneBridge4 RH panel (Hum Mol Genet 1996 March;5(3):339-46 A radiation hybrid map of the human genome. Gyapay G, Schnmitt K, Fizames C, Jones H, Vega-Czarny N, Spillett D, Muselet D, Prud'Homme J F, Dib C, Auffray C, Morissette J, Weissenbach J, Goodfellow P N). To determine the chromosomal location of a gene using this panel, 93 PCRs are performed using primers designed from the gene of interest on RH DNAs. Each of these DNAs contains random human genomic fragments maintained in a hamster background (human/hamster hybrid cell lines). These PCRs result in 93 scores indicating the presence or absence of the PCR product of the gene of interest. These scores are compared with scores created using PCR products from genomic sequences of known location. This comparison is conducted at http://www.genome.wi.mit.edu/.

The polynucleotide sequences of the present invention are also valuable tools for tissue expression studies. Such studies allow the determination of expression patterns of polynucleotides of the present invention which may give an indication as to the expression patterns of the encoded polypeptides in tissues, by detecting the mRNAs that encode them. The techniques used are well known in the art and include in situ hydridization techniques to clones arrayed on a grid, such as cDNA microarray hybridization (Schena et al, Science, 270, 467-470, 1995 and Shalon et al, Genome Res, 6, 639-645, 1996) and nucleotide amplification techniques such as PCR. A preferred method uses the TAQMAN (Trade mark) technology available from Perkin Elmer. Results from these studies can provide an indication of the normal function of the polypeptide in the organism. In addition, comparative studies of the normal expression pattern of mRNAs with that of mRNAs encoded by an alternative form of the same gene (for example, one having an alteration in polypeptide coding potential or a regulatory mutation) can provide valuable insights into the role of the polypeptides of the present invention, or that of inappropriate expression thereof in disease. Such inappropriate expression may be of a temporal, spatial or simply quantitative nature.

A further aspect of the present invention relates to antibodies. The polypeptides of the invention or their fragments, or cells expressing them, can be used as immunogens to produce antibodies that are immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

The above-described antibodies may be employed to isolate or to identify clones expressing the polypeptide or to purify the polypeptides by affinity chromatography. Antibodies against polypeptides of the present invention may also be employed to treat diseases of the invention, amongst others.

Polypeptides and polynucleotides of the present invention may also be used as vaccines. Accordingly, in a further aspect, the present invention relates to a method for inducing an immunological response in a mammal that comprises inoculating the mammal with a polypeptide of the present invention, adequate to produce antibody and/or T cell immune response, including, for example, cytokine-producing T cells or cytotoxic T cells, to protect said animal from disease, whether that disease is already established within the individual or not. An immunological response in a mammal may also be induced by a method comprises delivering a polypeptide of the present invention via a vector directing expression of the polynucleotide and coding for the polypeptide in vivo in order to induce such an immunological response to produce antibody to protect said animal from diseases of the invention. One way of administering the vector is by accelerating it into the desired cells as a coating on particles or otherwise. Such nucleic acid vector may comprise DNA, RNA, a modified nucleic acid, or a DNA/RNA hybrid. For use a vaccine, a polypeptide or a nucleic acid vector will be normally provided as a vaccine formulation (composition). The formulation may further comprise a suitable carrier. Since a polypeptide may be broken down in the stomach, it is preferably administered parenterally (for instance, subcutaneous, intramuscular, intravenous, or intra-dermal injection). Formulations suitable for parenteral administration include aqueous and non-aqueous sterile injection solutions that may contain anti-oxidants, buffers, bacteriostats and solutes that render the formulation instonic with the blood of the recipient; and aqueous and non-aqueous sterile suspensions that may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention have one or more biological functions that are of relevance in one or more disease states, in particular the diseases of the invention hereinbefore mentioned. It is therefore useful to identify compounds that stimulate or inhibit the function or level of the polypeptide. Accordingly, in a further aspect, the present invention provides for a method of screening compounds to identify those that stimulate or inhibit the function or level of the polypeptide. Such methods identify agonists or antagonists that may be employed for therapeutic and prophylactic purposes for such diseases of the invention as hereinbefore mentioned. Compounds may be identified from a variety of sources, for example, cells, cell-free preparations, chemical libraries, collections of chemical compounds, and natural product mixtures. Such agonists or antagonists so-identified may be natural or modified substrates, ligands, receptors, enzymes, etc., as the case may be, of the polypeptide; a structural or functional mimetic thereof (see Coligan et al., Current Protocols in Immunology 1(2):Chapter 5 (1991)) or a small molecule. Such small molecules preferably have a molecular weight below 2,000 daltons, more preferably between 300 and 1,000 daltons, and most preferably between 400 and 700 daltons. It is preferred that these small molecules are organic molecules.

The screening method may simply measure the binding of a candidate compound to the polypeptide, or to cells or membranes bearing the polypeptide, or a fusion protein thereof, by means of a label directly or indirectly associated with the candidate compound. Alternatively, the screening method may involve measuring or detecting (qualitatively or quantitatively) the competitive binding of a candidate compound to the polypeptide against a labeled competitor (e.g. agonist or antagonist). Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Further, the screening methods may simply comprise the steps of mixing a candidate compound with a solution containing a polypeptide of the present invention, to form a mixture, measuring an activity of the genes set forth in Table I in the mixture, and comparing activity of the mixture of the genes set forth in Table I to a control mixture which contains no candidate compound.

Polypeptides of the present invention may be employed in conventional low capacity screening methods and also in high-throughput screening (HTS) formats. Such HTS formats include not only the well-established use of 96- and, more recently, 384-well micotiter plates but also emerging methods such as the nanowell method described by Schullek et al, Anal Biochem., 246, 20-29, (1997).

Fusion proteins, such as those made from Fc portion and polypeptide of the genes set forth in Table I, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

A polypeptide of the present invention may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide that compete with the binding of the polypeptide to its receptors, if any. Standard methods for conducting such assays are well understood in the art.

Examples of antagonists of polypeptides of the present invention include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or a small molecule that bind to the polypeptide of the present invention but do not elicit a response, so that the activity of the polypeptide is prevented.

Screening methods may also involve the use of transgenic technology and the genes set forth in Table I. The art of constructing transgenic animals is well established. For example, the genes set forth in Table I may be introduced through microinjection into the male pronucleus of fertilized oocytes, retroviral transfer into pre- or post-implantation embryos, or injection of genetically modified, such as by electroporation, embryonic stem cells into host blastocysts. Particularly useful transgenic animals are so-called “knock-in” animals in which an animal gene is replaced by the human equivalent within the genome of that animal. Knock-in transgenic animals are useful in the drug discovery process, for target validation, where the compound is specific for the human target. Other useful transgenic animals are so-called “knock-out” animals in which the expression of the animal ortholog of a polypeptide of the present invention and encoded by an endogenous DNA sequence in a cell is partially or completely annulled. The gene knock-out may be targeted to specific cells or tissues, may occur only in certain cells or tissues as a consequence of the limitations of the technology, or may occur in all, or substantially all, cells in the animal. Transgenic animal technology also offers a whole animal expression-cloning system in which introduced genes are expressed to give large amounts of polypeptides of the present invention.

Screening kits for use in the above described methods form a further aspect of the present invention. Such screening kits comprise:

(a) a polypeptide of the present invention;

(b) a recombinant cell expressing a polypeptide of the present invention;

(c) a cell membrane expressing a polypeptide of the present invention; or

(d) an antibody to a polypeptide of the present invention;

which polypeptide is preferably that set forth in the Sequence Listing.

It will be appreciated that in any such kit, (a), (b), (c) or (d) may comprise a substantial component.

Glossary

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an

Fab or other immunoglobulin expression library.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Secreted protein activity or secreted polypeptide activity” or “biological activity of the secreted protein or secreted polypeptide” refers to the metabolic or physiologic function of said secreted protein including similar activities or improved activities or these activities with decreased undesirable side-effects. Also included are antigenic and immunogenic activities of said secreted protein.

“Secreted protein gene” refers to a polynucleotide comprising any of the attached nucleotide sequences or allelic variants thereof and/or their complements.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, 1-12, in Post-translational Covalent Modification of Proteins, B. C. Johnson, Ed., Academic Press, New York, 1983; Seifter et al., “Analysis for protein modifications and nonprotein cofactors”, Meth Enzymol, 182, 626-646, 1990, and Rattan et al., “Protein Synthesis: Post-translational Modifications and Aging”, Ann NY Acad Sci, 663, 48-62, 1992).

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucleotide sequence that is shorter than the reference sequence set forth in the Sequence Listing.

“Variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln; Ser, Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required. A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule each of that may encode different amino acid sequences. The term splice variant also refers to the proteins encoded by the above cDNA molecules.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“% Identity”—For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

“Similarity” is a further, more sophisticated measure of the relationship between two polypeptide sequences. In general, “similarity” means a comparison between the amino acids of two polypeptide chains, on a residue by residue basis, taking into account not only exact correspondences between a between pairs of residues, one from each of the sequences being compared (as for identity) but also, where there is not an exact correspondence, whether, on an evolutionary basis, one residue is a likely substitute for the other. This likelihood has an associated “score” from which the “% similarity” of the two sequences can then be determined.

Methods for comparing the identity and similarity of two or more sequences are well known in the art. Thus for instance, programs available in the Wisconsin Sequence Analysis Package, version 9.1 (Devereux J et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J Mol Biol, 147,195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J Mol Biol, 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S F et al, J Mol Biol, 215, 403-410, 1990, Altschul S F et al, Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183, 63-99, 1990; Pearson W R and Lipman D J, Proc Nat Acad Sci USA, 85, 2444-2448,1988, available as part of the Wisconsin Sequence Analysis Package).

Preferably, the BLOSUM62 amino acid substitution matrix (Henikoff S and Henikoff J G, Proc. Nat. Acad Sci. USA, 89, 10915-10919, 1992) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a reference polynucleotide or a polypeptide sequence, the query and the reference sequence being optimally aligned and the parameters of the program set at the default value, as hereinbefore described.

“Identity Index” is a measure of sequence relatedness which may be used to compare a candidate sequence (polynucleotide or polypeptide) and a reference sequence. Thus, for instance, a candidate polynucleotide sequence having, for example, an Identity Index of 0.95 compared to a reference polynucleotide sequence is identical to the reference sequence except that the candidate polynucleotide sequence may include on average up to five differences per each 100 nucleotides of the reference sequence. Such differences are selected from the group consisting of at least one nucleotide deletion, substitution, including transition and transversion, or insertion. These differences may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having an Identity Index of 0.95 compared to a reference polynucleotide sequence, an average of up to 5 in every 100 of the nucleotides of the in the reference sequence may be deleted, substituted or inserted, or any combination thereof, as hereinbefore described. The same applies mutatis mutatidis for other values of the Identity Index, for instance 0.96, 0.97, 0.98 and 0.99.

Similarly, for a polypeptide, a candidate polypeptide sequence having, for example, an Identity Index of 0.95 compared to a reference polypeptide sequence is identical to the reference sequence except that the polypeptide sequence may include an average of up to five differences per each 100 amino acids of the reference sequence. Such differences are selected from the group consisting of at least one amino acid deletion, substitution, including conservative and non-conservative substitution, or insertion. These differences may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a polypeptide sequence having an Identity Index of 0.95 compared to a reference polypeptide sequence, an average of up to 5 in every 100 of the amino acids in the reference sequence may be deleted, substituted or inserted, or any combination thereof, as hereinbefore described. The same applies mutatis mutatidis for other values of the Identity Index, for instance 0.96, 0.97, 0.98 and 0.99.

The relationship between the number of nucleotide or amino acid differences and the Identity Index may be expressed in the following equation:

nₐ≦xₐ−(xₐ•I),

in which:

nₐ is the number of nucleotide or amino acid differences,

xₐ is the total number of nucleotides or amino acids in a sequence set forth in the Sequence Listing,

I is the Identity Index,

• is the symbol for the multiplication operator, and

in which any non-integer product of xₐ and I is rounded down to the nearest integer prior to subtracting it from xₐ.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide that within the same species which is functionally similar.

“Fusion protein” refers to a protein encoded by two, often unrelated, fused genes or fragments thereof. In one example, EP-A-0 464 533-A discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

TABLE I

| | | | Corresponding |
|---|---|---|---|
| | GSK | Nucleic Acid | Protein |
| Gene Name | Gene ID | SEQ ID NO's | SEQ ID NO's |
| sbg237163LIPASE | 237163 | SEQ ID NO: 1 | SEQ ID NO: 23 |
| sbg251170CEAa | 251170 | SEQ ID NO: 2 | SEQ ID NO: 24 |
| | | SEQ ID NO: 3 | SEQ ID NO: 25 |
| sbg389686WNT15a | 389686 | SEQ ID NO: 4 | SEQ ID NO: 26 |
| | | SEQ ID NO: 5 | SEQ ID NO: 27 |
| sbg236015LIPASE | 236015 | SEQ ID NO: 6 | SEQ ID NO: 28 |
| | | SEQ ID NO: 7 | SEQ ID NO: 29 |
| sbg417005 | 417005 | SEQ ID NO: 8 | SEQ ID NO: 30 |
| LAMININ_ALPHA | | SEQ ID NO: 9 | SEQ ID NO: 31 |
| sbg425649KINASEa | 425649 | SEQ ID NO: 10 | SEQ ID NO: 32 |
| sbg419582 | 419582 | SEQ ID NO: 11 | SEQ ID NO: 33 |
| PROTOCADHERIN | | SEQ ID NO: 12 | SEQ ID NO: 34 |
| sbg453915 | 453915 | SEQ ID NO: 13 | SEQ ID NO: 35 |
| TECTORINa | | | |
| SBh385630.antiinflam | 385630 | SEQ ID NO: 14 | SEQ ID NO: 36 |
| | | SEQ ID NO: 15 | SEQ ID NO: 37 |
| sbg471005nAChR | 471005 | SEQ ID NO: 16 | SEQ ID NO: 38 |
| sbg442445PROa | 442445 | SEQ ID NO: 17 | SEQ ID NO: 39 |
| sbg456548CytoRa | 456548 | SEQ ID NO: 18 | SEQ ID NO: 40 |
| | | SEQ ID NO: 19 | SEQ ID NO: 41 |
| sbg456548CytoRa | 456548b | SEQ ID NO: 20 | SEQ ID NO: 42 |
| sbg442358PROa | 442358 | SEQ ID NO: 21 | SEQ ID NO: 43 |
| | | SEQ ID NO: 22 | SEQ ID NO: 44 |

TABLE II

| | | | | Cell |
|---|---|---|---|---|
| | | | | Localization |
| | Gene | Closest Polynuclotide | Closest Polypeptide by | (by |
| Gene Name | Family | by homology | homology | homology) |
| sbg237163 | Pancreatic | GB: AC011328 | Mouse pancreatic lipase | Secreted |
| LIPASE | lipase | Direct submitted (OCT- | related protein 1, gi: | |
| | | 06-1999) Genome | 9256628 | |
| | | Therapeutics | Remington, S. G., | |
| | | Corporation, 100 | Lima, P. H. and Nelson, J. D. | |
| | | Beaver Street, | Invest. Ophthalmol. Vis. | |
| | | Waltham, MA 02453, | Sci. 40 (6), 1081-1090 | |
| | | USA | (1999) | |
| sbg251170CEAa | Carcinoem | GB: AC020914 | Mouse putative protein, | Secreted |
| | bryonic | Submitted (JAN-12- | gi: 12842545 | |
| | antigen | 2000) Production | Carninci, P., Shibata, Y., | |
| | | Sequencing Facility, | Hayatsu, N., Sugahara, Y., | |
| | | DOE Joint | Shibata, K., Itoh, M., | |
| | | Genome Institute, 2800 | Konno, H., Okazaki, Y., | |
| | | Mitchell Drive, Walnut | Muramatsu, M. and | |
| | | Creek, CA 94598, USA | Hayashizaki, Y. | |
| | | | Genome Res. 10 (10), | |
| | | | 1617-1630 (2000). | |
| sbg389686 | WNT15 | GB: AC015855 | Chicken WNT14 protein, | Secreted |
| WNT15a | | Directly submitted (NOV- | gi: 3915306 | |
| | | 17-1999) Whitehead | Bergstein I, Eisenberg L M, | |
| | | Institute/MIT Center | Bhalerao J, Jenkins N A, | |
| | | for Genome Research, | Copeland N G, Osborne | |
| | | 320 Charles Street, | M P, Bowcock A M, Brown | |
| | | Cambridge, MA 02141, | A M; 1997; Genomics | |
| | | USA. | 46: 450-8. | |
| sbg236015LIPASE | Lysosomal | GB: AL358532 | Rat lingual lipase, | Secreted |
| | acid | Directly submitted (DEC- | gi: 126307 | |
| | lipase | 15-2000) by Sanger | Docherty, A. J., | |
| | | Centre, Hinxton, | Bodmer, M. W., Angal, S., | |
| | | Cambridgeshire, CB 10 | Verger, R., Riviere, C., | |
| | | 1SA, UK. | Lowe, P. A., Lyons, A., | |
| | | | Emtage, J. S. and Harris, T. J. | |
| | | | Nucleic Acids Res. 13 (6), | |
| | | | 1891-1903 (1985) | |
| sbg417005LAMININ_{—} | Laminin | GB: AL354836 | Human laminin alpha 5, | Secreted |
| ALPHA | alpha | Direct submitted (MAY- | gi: 12274842 | |
| | | 02-2000) Sanger | Submitted (FEB-14-2001) | |
| | | Centre, Hinxton, | by Sanger Centre, Hinxton, | |
| | | Cambridgeshire, CB 10 | Cambridgeshire, CB 10 | |
| | | 1SA | 1SA, UK. | |
| sbg425649KINASEa | Casein | GB: AL356107 | Human casein kinase I- | Cytosolic |
| | kinase I- | Submitted (MAY-16- | alpha, | |
| | alpha | 2000) by | gi: 2134872 | |
| | | Sanger Centre, | Fish, K. J., | |
| | | Hinxton, | Cegielska, A., | |
| | | Cambridgeshire, CB 10 | Getman, M. E., | |
| | | 1SA, UK. | Landes, G. M. and | |
| | | | Virshup, D. M. | |
| | | | J. Biol. Chem. 270 (25), | |
| | | | 14875-14883 (1995) | |
| sbg419582PROTOCADHERIN | Protocadherin | GB: AL355593 | Human protocadherin 68 | Secreted |
| | | Direct submitted (MAY- | gi: 11433373 | |
| | | 17-2000) Sanger | Submitted (NOV-16-2000) | |
| | | Centre, Hinxton, | by National Center for | |
| | | Cambridgeshire, CB 10 | Biotechnology | |
| | | 1SA, UK. | Information, NIH, | |
| | | | Bethesda, MD 20894, USA | |
| sbg453915TECTORINa | Tectorin | SC: AL157786 | Mouse tectorin beta, | Secreted |
| | Beta | Submitted (MAY-04- | gi: 7363457 | |
| | | 2001) by Sanger | Legan, P. K., Rau, A., | |
| | | Centre, Hinxton, | Keen, J. N. and | |
| | | Cambridgeshire, CB 10 | Richardson, G. P. | |
| | | 1SA, UK. | J. Biol. Chem. 272 (13), | |
| | | | 8791-8801 (1997) | |
| SBh385630. | Lipase | GB: AC015525 | Rabbit lacrimal lipase, | Secreted |
| antiinflam | | Submitted (NOV-16- | gi: 13560884 | |
| | | 1999) by Whitehead | Submitted (FEB-20-2001) | |
| | | Institute/MIT Center | Ophthalmology, Regions | |
| | | for Genome Research, | Hospital, 640 Jackson | |
| | | 320 Charles Street, | Street, St. Paul, MN 55101, | |
| | | Cambridge, MA 02141, | USA | |
| | | USA | | |
| sbg471005nAChR | Nicotinic | GB: AC060812 | Human cholinergic | Membrane- |
| | acetylcholine | Direct submitted | receptor, nicotinic, alpha | bound |
| | receptor | (APR-20-2000) | polypeptide 10, | |
| | | Whitehead | gi: 11138123 | |
| | | Institute/MIT | Lustig, L. R., Peng, H., | |
| | | Center for | Hiel, H., Yamamoto, T. | |
| | | Genome | and Fuchs, P. A. | |
| | | Research, 320 | Genomics 73 (3), 272- | |
| | | Charles Street, | 283 (2001) | |
| | | Cambridge, MA | | |
| | | 02141, USA | | |
| sbg442445PROa | Leucine rich | GB: AC060234 | RIKEN cDNA mouse | Cytosolic |
| | repeat protein | Submitted | 4930442L21 gene | |
| | | (APR-20-2000) | Carninci, P., Shibata, Y., | |
| | | Genome | Hayatsu, N., | |
| | | Therapeutics | Sugahara, Y., Shibata, K., | |
| | | Corporation, 100 | Itoh, M., Konno, H., | |
| | | Beaver Street, | Okazaki, Y., | |
| | | Waltham, | Muramatsu, M. and | |
| | | MA 02453, USA | Hayashizaki, Y. | |
| | | | Genome Res. 10 (10), | |
| | | | 1617-1630 (2000) | |
| sbg456548CytoRa | Cytokine | GB: AL158138 | Human IL20 receptor, | Membrane- |
| | receptor | Submitted (JAN- | gi: 7657691 | bound |
| | | 20-2001) by | Xie M H, Aggarwal S, | |
| | | Sanger Centre, | Ho W H, Foster J, Zhang | |
| | | Hinxton, | Z, Stinson J, Wood W I, | |
| | | Cambridgeshire, | Goddard AD and Gurney | |
| | | CB 10 1SA, UK. | A L. | |
| | | | J. Biol. Chem. 275 (40), | |
| | | | 31335-31339 (2000) | |
| sbg442358PROa | Leucine rich | GB: AL139099 | Human EXMAD-9 | Membrane- |
| | repeat protein | Submitted (MAY- | geneseqp: AAB27231 | bound |
| | | 23-2000) by | Submitted by INCYTE | |
| | | Genoscope- | GENOMICS INC | |
| | | Centre National | Application and | |
| | | de Sequencage: | publication date: | |
| | | BP 191 91006 | WO200068380-A2, NOV- | |
| | | EVRY cedex — | 16-00 | |
| | | FRANCE | | |

TABLE III

| | | Associated |
|---|---|---|
| Gene Name | Uses | Diseases |
| sbg237163 | An embodiment of the invention is the use of sbg237163 | Cancer, infection, |
| LIPASE | LIPASE as replacement enzymes for patients with chronic | autoimmune |
| | pancreatitis. A close homologue of sbg237163 LIPASE | disorder, |
| | is pancreatic lipase. Pancreatic lipase hydrolyzes dietary | hematopoietic |
| | long chain triacylglycerol to free fatty acids and | disorder, wound |
| | monoacylglycerols in the intestinal lumen (Lowe M E, | healing disorders, |
| | Rosenblum J L, and Strauss A W; 1989; J Biol Chem | inflammation. |
| | 264: 20042-8). Pancreatic steatorrhea and pancreatic | |
| | diabetes are the dominant symptoms of patients in a | |
| | certain stage of chronic pancreatitis. In this stage, the | |
| | nutritional state is greatly disturbed and hypoglycemia and | |
| | labile infection are involved. Pancreatic enzyme | |
| | replacement therapy is the principal treatment method for | |
| | pancreatic steatorrhea (Nakamura T, Takeuchi T, and | |
| | Tando Y; 1998; Pancreas 16: 329-36. | |
| sbg251170CEAa | An embodiment of the invention is the use of | Cancer, |
| | sbg251170CEAa as cell-surface molecules mediating | autoimmune |
| | cell-specific interactions in normal and neoplastic cells. A | disorders, wound |
| | close homologue of sbg251170CEAa is | healing disorders, |
| | carcinoembryonic antigen-related cell adhesion molecule | hematopoietic |
| | 6. Carcinoembryonic antigen-related cell adhesion | disorders and |
| | molecule 6 is claimed to function as a cell-surface | infection |
| | molecules mediating cell-specific interactions in normal | |
| | and neoplastic cells (1. Barnett T, Goebel S J, Nothdurft | |
| | M A, Elting J J, Carcinoembryonic antigen family: | |
| | characterization of cDNAs coding for NCA and CEA and | |
| | suggestion of nonrandom sequence variation in their | |
| | conserved loop-domains. Genomics 1988 Jul; 3(1): 59-66. | |
| | 2. Inazawa J, Abe T, Inoue K, Misawa S, Oikawa S, | |
| | Nakazato H, Yoshida M C. Regional assignment of | |
| | nonspecific cross-reacting antigen (NCA) of the CEA | |
| | gene family to chromosome 19 at band q13.2. Cytogenet | |
| | Cell Genet 1989; 52(1-2): 28-31). | |
| sbg389686 | An embodiment of the invention is the use of | Cancer, infection, |
| WNT15a | sbg389686WNT15a in regulation of cell growth and | autoimmune |
| | differentiation. Close homologues of | disorder, |
| | sbg389686WNT15a are Wnt proteins. Wnt proteins are | hematopoietic |
| | involved in critical developmental processes in both | disorder, wound |
| | vertebrates and invertebrates and are implicated in | healing disorders, |
| | regulation of cell growth and differentiation in certain | and inflammation |
| | adult mammalian tissues (Bergstein I, Eisenberg L M, | |
| | Bhalerao J, Jenkins N A, Copeland N G, Osborne M P, | |
| | Bowcock A M, Brown A M; 1997; Genomics 46: 450-8). | |
| | The Wnt gene family consists of at least 15 structurally | |
| | related genes that encode secreted extracellular | |
| | signaling factors. Wnt signaling is involved in many | |
| | mammalian developmental processes, including cell | |
| | proliferation, differentiation and epithelial-mesenchymal | |
| | interactions, through which they contribute to the | |
| | development of tissues and organs such as the limbs, the | |
| | brain, the reproductive tract and the kidney. Evidence | |
| | from tumor expression studies and transgenic animals | |
| | experiments suggests that inappropriate activation of the | |
| | Wnt signaling pathway is a major feature in human | |
| | neoplasia and that oncogenic activation of this pathway | |
| | can occur at many levels. Inappropriate expression of | |
| | the Wnt ligand and Wnt binding proteins have been | |
| | found in a variety of human tumors (Smalley M J, Dale | |
| | T C; 1999; Cancer Metastasis Rev 18: 215-30). | |
| sbg236015LIPASE | An embodiment of the invention is the use of | Cancer, infection, |
| | sbg236015LIPASE for treating lipase deficiency. A | autoimmune |
| | close homologue of sbg236015LIPASE is lysosomal | disorder, |
| | acid lipase. The lysosomal acid lipase catalyzes the | hematopoietic |
| | deacylation of triacylglyceryl and cholesteryl ester core | disorder, wound |
| | lipids of endocytosed low density lipoproteins. This | healing disorders, |
| | activity is deficient in patients with Wolman disease and | inflammation, |
| | cholesteryl ester storage disease, which are caused by a | Wolman disease, |
| | deficiency of lysosomal acid lipase activity, resulting in | and cholesteryl |
| | massive accumulation of cholesteryl ester and | ester storage |
| | triglycerides (Anderson R A, Sando G N; 1991; J Biol | disease |
| | Chem 266: 22479-84). | |
| sbg417005LAMININ_{—} | An embodiment of the invention is the use of | Cancer, infection, |
| ALPHA | sbg417005LAMININ_ALPHA to promote myogenesis | autoimmune |
| | in skeletal muscle, outgrowth of neurites from central | disorder, |
| | and peripheral neurons, and mesenchymal to epithelial | hematopoietic |
| | transitions in kidney. A close homologue of | disorder, wound |
| | sbg417005LAMININ_ALPHA is laminin. Laminins | healing disorders, |
| | trimers, composed of alpha, beta, and gamma chains, are | inflammation, |
| | components of all basal laminae (BLs) throughout the | congenital |
| | bodies. In mammals they play at least three essential | muscular |
| | roles. First, they are major structural elements of BLs, | dystrophy, and |
| | forming one of two self-assembling networks to which | junctional |
| | other glycoproteins and proteoglycans of the BL attach. | epidermolysis |
| | Second, they interact with cell surface components such | bullosa |
| | as dystroglycan to attach cells to the extracellular | |
| | matrix. Third, they are signaling molecules that interact | |
| | with cellular receptors such as the integrins to convey | |
| | important information to the cell interior. The alpha | |
| | chains are ligands for most cellular laminin receptors. | |
| | (Miner J H, Patton B L, Lentz S I, Gilbert D J, Snider W D, | |
| | Jenkins N A, Copeland N G, Sanes J R; 1997; J Cell Biol | |
| | 137: 685-701). | |
| sbg425649KINASEa | An embodiment of the invention is the use of | Cancer, wound |
| | sbg425649KINASEa in DNA replication and repair, | healing disorders, |
| | membrane trafficking, neuroprotective, cytostatic, | autoimmune |
| | cardioactive, immunomodulatory, muscular, vulnerary, | disorders, |
| | gastrointestinal, nephrotropic, anti-infective, | hematopoietic |
| | gynaecological and antibacterial activities, and can be | disorders and |
| | used in gene therapy. Close homologues of | infection |
| | sbg425649KINASEa is mammalian casein kinases I | |
| | (CKI) and human prostate cancer associated protein. | |
| | CKI belongs to a family of serine/threonine protein | |
| | kinases involved in diverse cellular processes including | |
| | DNA replication and repair, membrane trafficking, | |
| | circadian rhythms and Wnt signaling. Human prostate | |
| | cancer associated proteins have neuroprotective, | |
| | cytostatic, cardioactive, immunomodulatory, muscular, | |
| | vulnerary, gastrointestinal, nephrotropic, anti-infective, | |
| | gynaecological and antibacterial activities, and can be | |
| | used in gene therapy. | |
| sbg419582PROTOCADHERIN | An embodiment of the invention is the use of | Cancer, infection, |
| | sbg419582PROTOCADHERIN in functional systems of | autoimmune |
| | the nervous system, and may be involved in the | disorder, |
| | formation of the neural network. A close homologue of | hematopoietic |
| | sbg419582PROTOCADHERIN is protocadherin. The | disorder, wound |
| | expression of protocadherin is developmentally | healing disorders, |
| | regulated in a subset of the functional systems of the | inflammation, |
| | nervous system, and may be involved in the formation | Parkinson's |
| | of the neural network by segregation of the brain nuclei | disease, |
| | and mediation of the axonal connections (Hirano S, Yan | Huntington's |
| | Q, Suzuki S T; 1999; J Neurosci 19: 995-1005). The | chorea, and |
| | members of the cadherin superfamily are divided into | multiple sclerosis |
| | two groups: classical cadherin type and protocadherin | |
| | type. The current cadherins appear to have evolved from | |
| | protocadherin (Suzuki S T; 1996; J Cell Sci 109: 2609-11). | |
| sbg453915TECTORINa | An embodiment of the invention is the use of | Infection, cancer, |
| | sbg453915TECTORINa, a secreted protein, in cellular | wound healing |
| | adhesion. A close homologue of | disorders, |
| | sbg453915TECTORINa is mouse tectorin beta. The | hemotopoietic |
| | beta-tectorin is a protein of 36,074 Da that contains 4 | disorders and |
| | consensus N glycosylation sites and a single zona | autoimmune |
| | pellucida domain. It is similar to components of the | disorders. |
| | sperm-egg adhesion system, and, as such may have a | |
| | similar functional role (Legan P K, Rau A, Keen J N, | |
| | Richardson G P, The mouse tectorins. Modular matrix | |
| | proteins of the inner ear homologous to components of | |
| | the sperm-egg adhesion system. J Biol Chem 1997 Mar | |
| | 28; 272(13): 8791-801). | |
| SBh385630. | An embodiment of the invention is the use of | Lematopoietic |
| antiinflam | SBh385630.antiinflam in gene therapy and are also | disorders, wound |
| | suggested to have cytokine and cell | healing disorders, |
| | proliferation/differentiation activity, immune | viral and bacterial |
| | stimulating (e.g. vaccines) or suppressing activity, | infections, cancer, |
| | haematopoiesis regulating activity, tissue growth | and autoimmune |
| | activity, activin/inhibinactivity, | diseases |
| | chemotactic/chemokinetic activity, haemostatic and | |
| | thrombolytic activity, receptor/ligand activity, anti- | |
| | inflammatory activity, cadherin/tumour invasion | |
| | suppressor activity, and tumour inhibition activity. | |
| | Lipases are also reported to be useful for gene therapy | |
| | (WO9957132-A1; Agostino, M. J., filed by GENETICS | |
| | INST INC.). Close homologues of | |
| | SBh385630.antiinflam include lipases. | |
| sbg471005n | An embodiment of the invention is the use of | Cancer, infection, |
| AChR | sbg471005nAChR in physiological and behavioural | autoimmune |
| | processes of the brain. A close homologue of | disorder, |
| | sbg471005nAChR is neuronal nicotinic acetylcholine | hematopoietic |
| | receptors. Neuronal nicotinic acetylcholine receptors | disorder, wound |
| | are a family of ion channels which are widely | healing disorders, |
| | distributed in the human brain. There are many | inflammation, |
| | subtypes, and each has individual pharmacological and | Alzheimer's |
| | functional profiles. They mediate the effects of nicotine, | disease, |
| | and are involved in a number of physiological and | Parkinson's |
| | behavioural processes. Additionally they may be | disease, and |
| | implicated in a number of pathological conditions such | schizophrenia |
| | as Alzheimer's disease, Parkinson's disease and | |
| | schizophrenia (Paterson D, Nordberg A; 2000; Prog | |
| | Neurobiol 61: 75-111). | |
| sbg442445PROa | An embodiment of the invention is the use of | Inflammation, |
| | sbg442445PROa which may be involved in protein- | autoimmune |
| | protein interation and signal transduction in immune | disorders, asthma, |
| | system. sbg442445PROa was expressed predominantly | allergies |
| | in lung and spleen/lymph. It encodes a protein with | and |
| | leucine rich repeats which may be involved in protein- | sbg442445PROa- |
| | protein interation and signal transduction in immune | associated |
| | systems. | disorders |
| sbg456548CytoRa | The present gene has been cloned. Sybrman data | Chronic and acute |
| | showed its high expression levels in placenta and | inflammation, |
| | moderate levels in spleen and lymph. A close | allergy, arthritis |
| | homologue of sbg456548CytoRa is another Class II | (including |
| | cytokine receptor, ZCYTOR7. An embodiment of the | rheumatoid |
| | invention is the use of sbg456548CytoRa, a decoy | arthritis), |
| | receptor, in the identification of other ligands, the | septicemia, |
| | promotion of anti-microbial activation of these cells, | autoimmune |
| | and/or potentiate the effectiveness of the natural ligand. | diseases (e.g., |
| | Growth factors are known to promote the progression of | inflammatory |
| | cancer. A decoy receptor could interfere with that | bowel disease, |
| | process. Proliferation, survival and differentiation can | psoriasis), |
| | be transduced from activated cytokine receptors (Cell | transplant |
| | Signal. 1998. 10(9): 619-628). Blocking these events | rejection, graft vs. |
| | could be crucial in modulating various diseases. | host disease, |
| | The decoy receptor could potentially interfere with | infection, stroke, |
| | binding of these or other putative ligands, preventing | ischemia, acute |
| | downstream effects (Blood. 1999. 94(6): 1943-1951). | respiratory disease |
| | GM-CSF also has anti-apoptotic activity. A decoy | syndrome, asthma, |
| | receptor might then be able to block GM-CSF's anti- | restenosis, brain |
| | apoptotic actions when appropriate (Mol Biol Cell. | injury, AIDS, bone |
| | 1999. 10(11): 3959-3970). Roles for blocking the | diseases, cancer, |
| | activity of the decoy receptor can be envisioned. GM- | atheroschlerosis, |
| | CSF promotes anti-microbial functions of mature | Alzheimers |
| | neutrophils. Inhibiting the activity of an interfering | disease,, |
| | decoy receptor could promote anti-microbial activation | hematopoietic |
| | of these cells. Furthermore, rhGM-CSF is in wide | disorder, and |
| | clinical use to fight acute myeloid leukemia | wound healing |
| | (Haematologica. 1991. 82(2): 239-245). Inhibition of a | disorder |
| | decoy receptor could potentiate the effectiveness of the | |
| | natural ligand. | |
| sbg442358PROa | An embodiment of the invention is the use of | Cancer, |
| | sbg442358PROa useful in the prevention and treatment | autoimmune |
| | of cancers, cell proliferation, cardiovascular, | disorders, |
| | reproductive, immune, musculoskeletal, developmental | hemotopoietic |
| | and gastrointestinal disorders and inflammation. Close | disorders, wound |
| | homologues of sbg442358PROa are human protein | healing disorders |
| | B27231 and Drosophila LRR47 that also contains | and infections |
| | leucine-rich repeats (LRRs) motifs. LRR has been | |
| | found in a variety of extracellular, membrane and | |
| | cytoplasmic proteins and are believed to mediate | |
| | specific protein-protein interactions and to function in | |
| | cellular adhesion (Ntwasa, M., Buchanan, S. G. and | |
| | Gay, N. J. Biochim. Biophys. Acta 1218 (2), 181-186 | |
| | (1994)). | |

TABLE IV
Quantitative, Tissue-specific, mRNA expression detected using SybrMan

Quantitative, tissue-specific, mRNA expression patterns of the genes were measured using SYBR-
Green Quantitative PCR (Applied Biosystems, Foster City, CA; see Schmittgen T. D. et al.,
Analytical Biochemistry 285: 194-204, 2000) and human cDNAs prepared from various human
tissues. Gene-specific PCR primers were designed using the first nucleic acid sequence listed in the
Sequence List for each gene. Results are presented as the number of copies of each specific gene's
mRNA detected in 1 ng mRNA pool from each tissue. Two replicate mRNA measurements were
made from each tissue RNA.

Tissue-Specific mRNA Expression
(copies per ng mRNA; avg. ± range for 2 data points per tissue)

| | | | | | | | | | | |
|---|---|---|---|---|---|---|---|---|---|---|
| Gene | | | | | | Skeletal | | | | |
| Name | Brain | Heart | Lung | Liver | Kidney | muscle | Intestine | Spleen/lymph | Placenta | Testis |

Gene Name sbg237163LIPASE

| | | | | | | | | | | |
|---|---|---|---|---|---|---|---|---|---|---|
| sbg237163LIPASE | 5 ± 0 | 8 ± 2 | 7 ± 2 | −6 ± 1 | 5 ± 1 | 5 ± 2 | 4 ± 6 | 3 ± 2 | 1 ± 1 | 47 ± 1 |

Gene Name sbg251170CEAa

| | | | | | | | | | | |
|---|---|---|---|---|---|---|---|---|---|---|
| sbg251170CEAa | 3 ± 1 | 19 ± | 30 ± | −5 ± 3 | 3 ± 1 | 5 ± 5 | 21 ± 2 | 33 ± 4 | 22 ± 3 | 14 ± 0 |
| | | 1 | 5 | | | | | | | |

In each gene's first subset table, two replicate measurements of gene of identification (GOI) mRNA
were measured from various human tissues (column 2 and 3). The average GOI mRNA copies of
the two replicates were made from each tissue RNA (column 4). The average amount of 18S rRNA
from each tissue RNA was measured (column 5) and used for normalization. To make each tissue
with the same amount of 50 ng of 18S rRNA, the normalization factor (column 6) was calculated
by dividing 50 ng with the amount of 18S rRNA measured from each tissue (column 5). The
mRNA copies per 50 ng of total RNA were obtained by multipling each GOI normalization factor
and average mRNA copies (column7).
Fold changes shown in each gene's second subset table were only calculated for disease tissues
which have a normal counterpart. There are blanks in the fold change column for all samples that
do not have counterparts. In addition, the fold change calculations are the fold change in the disease
sample as compared to the normal sample. Accordingly, there will not be a fold change calculation
next to any of the normal samples. For patient matched cancer pairs (colon, lung, and breast), each
tumor is compared to its specific normal counterpart. When patient-matched normal/disease pairs
do not exist, each disease sample was compared back to the average of all the normal samples of
that same tissue type. For example, normal brain from the same patient that provided Alzheimer's
brain is not applicable. Three normal brain samples and 4 Alzheimer's brain samples are used in the
fold change. Three normal samples were averaged, and each of the Alzheimer's samples was
compared back to that average.
Abbreviations

| | |
|---|---|
| ALZ | Alzheimer's Disease |
| CT | CLONTECH (1020 East Meadow Circle Palo Alto, CA 94303-4230, USA) |
| KC | Sample prepared by GSK investigator |
| COPD | chronic obstructive pulmonary disease |
| endo | endothelial |
| VEGF | vascular endothelial growth factor |
| bFGF | basic fibroblast growth factor |
| BM | bone marrow |
| osteo | osteoblast |
| OA | osteoarthritis |
| RA | rheumatoid arthritis |
| PBL | peripheral blood lymphocytes |
| PBMNC | peripheral blood mononuclear cells |
| HIV | human immunodeficiency virus |
| HSV | Herpes simplex virus |
| HPV | human papilloma virus |

Gene Name sbg389686WNT15a
Strong expression in Brain and dendritic cells. Brain expression may be from presence of glial cells.
Expression in RA and OA synovium along with dendritic cells suggests a role for this protein in
these diseases. Down regulation in ischemic and dilated heart indicates that replacement of protein
could be therapeutic.

| | | | | | | copies of |
|---|---|---|---|---|---|---|
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg389686WNT15a | (sample 1) | (sample 2) | Copies | (ng) | (ng) | RNA |
| Subcutaneous | 0.00 | 0.00 | 0.00 | 3.06 | 16.34 | 0.00 |
| Adipocytes Zenbio | | | | | | |
| Subcutaneous Adipose | 0.00 | 1.71 | 0.86 | 0.96 | 52.36 | 44.76 |
| Zenbio | | | | | | |
| Adrenal Gland Clontech | 2.29 | 4.18 | 3.24 | 0.61 | 81.97 | 265.16 |
| Whole Brain Clontech | 698.52 | 625.01 | 661.77 | 7.24 | 6.91 | 4570.20 |
| Fetal Brain Clontech | 4.14 | 6.78 | 5.46 | 0.48 | 103.95 | 567.57 |
| Cerebellum Clontech | 2.02 | 3.63 | 2.83 | 2.17 | 23.04 | 65.09 |
| Cervix | 3.16 | 10.14 | 6.65 | 2.42 | 20.66 | 137.40 |
| Colon | 2.48 | 3.44 | 2.96 | 2.71 | 18.45 | 54.61 |
| Endometrium | 2.69 | 5.20 | 3.95 | 0.73 | 68.21 | 269.10 |
| Esophagus | 10.67 | 3.24 | 6.96 | 1.37 | 36.50 | 253.83 |
| Heart Clontech | 9.26 | 6.07 | 7.67 | 1.32 | 37.88 | 290.34 |
| Hypothalamus | 7.10 | 5.16 | 6.13 | 0.32 | 155.28 | 951.86 |
| Ileum | 2.04 | 10.37 | 6.21 | 2.58 | 19.38 | 120.25 |
| Jejunum | 36.78 | 27.16 | 31.97 | 6.60 | 7.58 | 242.20 |
| Kidney | 16.46 | 16.55 | 16.51 | 2.12 | 23.58 | 389.27 |
| Liver | 14.07 | 3.34 | 8.71 | 1.50 | 33.33 | 290.17 |
| Fetal Liver Clontech | 4.60 | 8.89 | 6.75 | 10.40 | 4.81 | 32.43 |
| Lung | 3.11 | 10.49 | 6.80 | 2.57 | 19.46 | 132.30 |
| Mammary Gland | 3.28 | 10.61 | 6.95 | 13.00 | 3.85 | 26.71 |
| Clontech | | | | | | |
| Myometrium | 1.79 | 13.84 | 7.82 | 2.34 | 21.37 | 166.99 |
| Omentum | 1.96 | 2.65 | 2.31 | 3.94 | 12.69 | 29.25 |
| Ovary | 4.50 | 1.71 | 3.11 | 4.34 | 11.52 | 35.77 |
| Pancreas | 3.40 | 2.41 | 2.91 | 0.81 | 61.80 | 179.54 |
| Head of Pancreas | 2.22 | 4.63 | 3.43 | 1.57 | 31.85 | 109.08 |
| Parotid Gland | 5.48 | 2.07 | 3.78 | 5.48 | 9.12 | 34.44 |
| Placenta Clontech | 15.15 | 12.80 | 13.98 | 5.26 | 9.51 | 132.84 |
| Prostate | 3.39 | 7.44 | 5.42 | 3.00 | 16.67 | 90.25 |
| Rectum | 2.98 | 3.94 | 3.46 | 1.23 | 40.65 | 140.65 |
| Salivary Gland | 3.24 | 1.61 | 2.43 | 7.31 | 6.84 | 16.59 |
| Clontech | | | | | | |
| Skeletal Muscle | 2.01 | 1.55 | 1.78 | 1.26 | 39.68 | 70.63 |
| Clontech | | | | | | |
| Skin | 2.69 | 3.45 | 3.07 | 1.21 | 41.32 | 126.86 |
| Small Intestine | 5.39 | 1.67 | 3.53 | 0.98 | 51.07 | 180.29 |
| Clontech | | | | | | |
| Spleen | 3.96 | 2.52 | 3.24 | 4.92 | 10.16 | 32.93 |
| Stomach | 1.08 | 5.33 | 3.21 | 2.73 | 18.32 | 58.70 |
| Testis Clontech | 3.27 | 2.88 | 3.08 | 0.57 | 87.87 | 270.21 |
| Thymus Clontech | 5.43 | 4.42 | 4.93 | 9.89 | 5.06 | 24.90 |
| Thyroid | 2.32 | 3.01 | 2.67 | 2.77 | 18.05 | 48.10 |
| Trachea Clontech | 1.64 | 4.25 | 2.95 | 9.71 | 5.15 | 15.16 |
| Urinary Bladder | 3.63 | 6.81 | 5.22 | 5.47 | 9.14 | 47.71 |
| Uterus | 31.55 | 11.10 | 21.33 | 5.34 | 9.36 | 199.67 |

| | | | copies of | | |
|---|---|---|---|---|---|
| | Reg | | mRNA | | |
| | number | Mean | detected/50 ng | | Fold Change in |
| Sample | (GSK | GOI | total | | Disease |
| sbg389686WNT15a | identifier) | copies | RNA | Sample | Population |
| colon normal GW98-167 | 21941 | 36.16 | 72.32 | colon normal | |
| colon tumor GW98-166 | 21940 | 71.5 | 143.00 | colon tumor | 1.977323009 |
| colon normal GW98-178 | 22080 | 2.09 | 4.18 | colon normal | |
| colon tumor GW98- 177 | 22060 | 9.84 | 19.68 | colon tumor | 4.708133971 |
| colon normal GW98-561 | 23514 | 13.09 | 26.18 | colon normal | |
| colon tumor GW98-560 | 23513 | 15.11 | 30.22 | colon tumor | 1.154316272 |
| colon normal GW98-894 | 24691 | 8.62 | 17.24 | colon normal | |
| colon tumor GW98-893 | 24690 | 5.76 | 11.52 | colon tumor | −1.496527778 |
| lung normal GW98-3 | 20742 | 140.19 | 280.38 | lung normal | |
| lung tumor GW98-2 | 20741 | 1.67 | 3.34 | lung tumor | −83.94610778 |
| lung normal GW97-179 | 20677 | 60.54 | 121.08 | lung normal | |
| lung tumor GW97- 178 | 20676 | 135.62 | 271.24 | lung tumor | 2.240171787 |
| lung normal GW98-165 | 21922 | 257.96 | 515.92 | lung normal | |
| lung tumor GW98-164 | 21921 | 61.69 | 123.38 | lung tumor | −4.181552926 |
| lung normal GW98-282 | 22584 | 49.3 | 98.60 | lung normal | |
| lung tumor GW98-281 | 22583 | 12.39 | 24.78 | lung tumor | −3.979015335 |
| breast normal GW00-392 | 28750 | 71.94 | 71.94 | breast | |
| | | | | normal | |
| breast tumor GW00-391 | 28746 | 41.4 | 82.80 | breast tumor | 1.150959133 |
| breast normal GW00-413 | 28798 | 19.37 | 19.37 | breast | |
| | | | | normal | |
| breast tumor GW00-412 | 28797 | 1.13 | 2.26 | breast tumor | −8.57079646 |
| breast normal GW00- | 27592-95 | 8.19 | 8.19 | breast | |
| 235:238 | | | | normal | |
| breast tumor GW00- | 27588-91 | 38.27 | 38.27 | breast tumor | 4.672771673 |
| 231:234 | | | | | |
| breast normal GW98-621 | 23656 | 77.26 | 154.52 | breast | |
| | | | | normal | |
| breast tumor GW98-620 | 23655 | 37.57 | 75.14 | breast tumor | −2.056428001 |
| brain normal BB99-542 | 25507 | 597.17 | 1194.34 | brain normal | |
| brain normal BB99-406 | 25509 | 104.34 | 208.68 | brain normal | |
| brain normal BB99-904 | 25546 | 282.15 | 564.30 | brain normal | |
| brain stage 5 ALZ BB99- | 25502 | 84.26 | 168.52 | brain stage 5 | −3.891367988 |
| 874 | | | | ALZ | |
| brain stage 5 ALZ BB99- | 25503 | 247.01 | 494.02 | brain stage 5 | −1.327422641 |
| 887 | | | | ALZ | |
| brain stage 5 ALZ BB99- | 25504 | 173.02 | 346.04 | brain stage 5 | −1.895079567 |
| 862 | | | | ALZ | |
| brain stage 5 ALZ BB99- | 25542 | 253.73 | 507.46 | brain stage 5 | −1.292266057 |
| 927 | | | | ALZ | |
| CT lung KC | normal | 146.22 | 292.44 | CT lung | |
| lung 26 KC | normal | 150.46 | 150.46 | lung 26 | |
| lung 27 KC | normal | 0 | 0.00 | lung 27 | |
| lung 24 KC | COPD | 4.76 | 4.76 | lung 24 | −23.36292017 |
| lung 28 KC | COPD | 10.06 | 10.06 | lung 28 | −11.05442346 |
| lung 23 KC | COPD | 2.75 | 2.75 | lung 23 | −40.43909091 |
| lung 25 KC | COPD | 1.93 | 1.93 | lung 25 | |
| asthmatic lung | 29321 | 20.88 | 20.88 | asthmatic | −5.326029693 |
| ODO3112 | | | | lung | |
| asthmatic lung | 29323 | 133.29 | 266.58 | asthmatic | 2.397140481 |
| ODO3433 | | | | lung | |
| asthmatic lung | 29322 | 322.77 | 645.54 | asthmatic | 5.804824315 |
| ODO3397 | | | | lung | |
| asthmatic lung | 29325 | 43.52 | 87.04 | asthmatic | −1.277659697 |
| ODO4928 | | | | lung | |
| endo cells KC | control | 1.89 | 1.89 | endo cells | |
| endo VEGF KC | | 0 | 0.00 | endo VEGF | −1.89 |
| endo bFGF KC | | 1.17 | 1.17 | endo bFGF | −1.615384615 |
| heart Clontech | normal | 153.9 | 307.80 | heart | |
| heart (T-1) ischemic | 29417 | 137.74 | 275.48 | heart T-1 | −1.117322492 |
| heart (T-14) non- | 29422 | 87.79 | 175.58 | heart T-14 | −1.753047044 |
| obstructive DCM | | | | | |
| heart (T-3399) DCM | 29426 | 43.68 | 87.36 | heart T-3399 | −3.523351648 |
| adenoid GW99-269 | 26162 | 17.62 | 35.24 | adenoid | |
| tonsil GW98-280 | 22582 | 52.34 | 104.68 | tonsil | |
| T cells PC00314 | 28453 | 8.45 | 16.90 | T cells | |
| PBMNC KC | | 1.99 | 1.99 | PBMNC | |
| monocyte KC | | 4.74 | 9.48 | monocyte | |
| B cells PC00665 | 28455 | 7.65 | 15.30 | B cells | |
| dendritic cells 28441 | | 194.97 | 389.94 | dendritic | |
| | | | | cells | |
| neutrophils | 28440 | 2.13 | 2.13 | neutrophils | |
| eosinophils | 28446 | 7.25 | 14.50 | eosinophils | |
| BM unstim KC | | 0 | 0.00 | BM unstim | |
| BM stim KC | | 0 | 0.00 | BM stim | 0 |
| osteo dif KC | | 1.48 | 1.48 | osteo dif | |
| osteo undif KC | | 7.41 | 7.41 | osteo undif | 5.006756757 |
| chondrocytes | | 26.64 | 66.60 | chondrocytes | |
| OA Synovium IP12/01 | 29462 | 476.3 | 476.30 | OA | |
| | | | | Synovium | |
| OA Synovium NP10/01 | 29461 | 151.36 | 302.72 | OA | |
| | | | | Synovium | |
| OA Synovium NP57/00 | 28464 | 165.01 | 330.02 | OA | |
| | | | | Synovium | |
| RA Synovium NP03/01 | 28466 | 84.02 | 168.04 | RA | |
| | | | | Synovium | |
| RA Synovium NP71/00 | 28467 | 184.75 | 369.50 | RA | |
| | | | | Synovium | |
| RA Synovium NP45/00 | 28475 | 223.3 | 446.60 | RA | |
| | | | | Synovium | |
| OA bone (biobank) | 29217 | 72.31 | 72.31 | OA bone | |
| | | | | (biobank) | |
| OA bone Sample 1 | J. Emory | 10.46 | 20.92 | OA bone | |
| OA bone Sample 2 | J. Emory | 111.79 | 223.58 | OA bone | |
| Cartilage (pool) | Normal | 215.54 | 431.08 | Cartilage | |
| | | | | (pool) | |
| Cartilage (pool) | OA | 81.85 | 163.70 | Cartilage | −2.633353696 |
| | | | | (pool) | |
| PBL unifected | 28441 | 2.31 | 4.62 | PBL | |
| | | | | unifected | |
| PBL HIV IIIB | 28442 | 2.28 | 4.56 | PBL HIV | −1.013157895 |
| | | | | IIIB | |
| MRC5 uninfected | 29158 | 2.37 | 4.74 | MRC5 | |
| (100%) | | | | uninfected | |
| | | | | (100%) | |
| MRC5 HSV strain F | 29178 | 37.5 | 75.00 | MRC5 HSV | 15.82278481 |
| | | | | strain F | |
| W12 cells | 29179 | 0.93 | 1.86 | W12 cells | |
| Keratinocytes | 29180 | 1.33 | 2.66 | Keratinocytes | |

Gene Name sbg389686WNT15a

| | Fold Change in Disease |
|---|---|
| | Population Relative to |
| Disease tissues | Normal |
| colon tumor | 1.98 |
| colon tumor | 4.71 |
| colon tumor | 1.15 |
| colon tumor | −1.50 |
| lung tumor | −83.95 |
| lung tumor | 2.24 |
| lung tumor | −4.18 |
| lung tumor | −3.98 |
| breast tumor | 1.15 |
| breast tumor | −8.57 |
| breast tumor | 4.67 |
| breast tumor | −2.06 |
| brain stage 5 ALZ | −3.89 |
| brain stage 5 ALZ | −1.33 |
| brain stage 5 ALZ | −1.90 |
| brain stage 5 ALZ | −1.29 |
| lung 24 | −23.36 |
| lung 28 | −11.05 |
| lung 23 | −40.44 |
| asthmatic lung | −5.33 |
| asthmatic lung | 2.40 |
| asthmatic lung | 5.80 |
| asthmatic lung | −1.28 |
| endo VEGF | −1.89 |
| endo bFGF | −1.62 |
| heart T-1 | −1.12 |
| heart T-14 | −1.75 |
| heart T-3399 | −3.52 |
| BM stim | 0.00 |
| osteo undif | 5.01 |
| Cartilage (pool) | −2.63 |
| PBL HIV IIIB | −1.01 |
| MRC5 HSV strain F | 15.82 |

Gene Name sbg236015LIPASE

Strongly expressed in neutrophils and eosinophils suggesting an immune system function. Additional expression is seen in RA and OA synovium and 1/3 OA bone samples. This suggests an involvement of 236015 in RA and OA. The high expression in skin when taken together with expression in neutrophils and eosinophils suggests possible involvement in immune pathologies of the skin ie. Eosinophilia, psoriasis and eczema. The expression in eosinophils also suggests involvement in allergic reactions. Expression in neutrophils suggests role in anti-infectives.

| | | | | | | copies of |
|---|---|---|---|---|---|---|
| | | | | | | mRNA |
| | | | | | 50 ng/ | detected/ |
| | Mean GOI | Mean GOI | Average | 18S | 18S | 50 ng |
| Sample | copies | copies | GOI | rRNA | rRNA | total |
| sbg236015LIPASE | (sample 1) | (sample 2) | Copies | (ng) | (ng) | RNA |
| Subcutaneous | 0.00 | 11.45 | 5.73 | 3.06 | 16.34 | 93.55 |
| Adipocytes Zenbio | | | | | | |
| Subcutaneous Adipose | 0.00 | 1.33 | 0.67 | 0.96 | 52.36 | 34.82 |
| Zenbio | | | | | | |
| Adrenal Gland Clontech | 0.52 | 5.04 | 2.78 | 0.61 | 81.97 | 227.87 |
| Whole Brain Clontech | 15.73 | 14.55 | 15.14 | 7.24 | 6.91 | 104.56 |
| Fetal Brain Clontech | 1.02 | 0.94 | 0.98 | 0.48 | 103.95 | 101.87 |
| Cerebellum Clontech | 0.38 | 0.39 | 0.39 | 2.17 | 23.04 | 8.87 |
| Cervix | 16.33 | 20.03 | 18.18 | 2.42 | 20.66 | 375.62 |
| Colon | 32.41 | 50.89 | 41.65 | 2.71 | 18.45 | 768.45 |
| Endometrium | 0.40 | 0.42 | 0.41 | 0.73 | 68.21 | 27.97 |
| Esophagus | 5.45 | 22.47 | 13.96 | 1.37 | 36.50 | 509.49 |
| Heart Clontech | 0.92 | 0.00 | 0.46 | 1.32 | 37.88 | 17.42 |
| Hypothalamus | 0.50 | 1.59 | 1.05 | 0.32 | 155.28 | 162.27 |
| Ileum | 41.95 | 1.51 | 21.73 | 2.58 | 19.38 | 421.12 |
| Jejunum | 7.59 | 15.40 | 11.50 | 6.60 | 7.58 | 87.08 |
| Kidney | 5.32 | 6.82 | 6.07 | 2.12 | 23.58 | 143.16 |
| Liver | 12.64 | 19.46 | 16.05 | 1.50 | 33.33 | 535.00 |
| Fetal Liver Clontech | 10.02 | 5.90 | 7.96 | 10.40 | 4.81 | 38.27 |
| Lung | 22.86 | 24.78 | 23.82 | 2.57 | 19.46 | 463.42 |
| Mammary Gland | 1.53 | 20.56 | 11.05 | 13.00 | 3.85 | 42.48 |
| Clontech | | | | | | |
| Myometrium | 16.05 | 1.34 | 8.70 | 2.34 | 21.37 | 185.79 |
| Omentum | 8.33 | 9.88 | 9.11 | 3.94 | 12.69 | 115.55 |
| Ovary | 8.22 | 14.40 | 11.31 | 4.34 | 11.52 | 130.30 |
| Pancreas | 0.00 | 1.58 | 0.79 | 0.81 | 61.80 | 48.83 |
| Head of Pancreas | 0.00 | 1.98 | 0.99 | 1.57 | 31.85 | 31.53 |
| Parotid Gland | 5.30 | 11.45 | 8.38 | 5.48 | 9.12 | 76.41 |
| Placenta Clontech | 11.93 | 1.22 | 6.58 | 5.26 | 9.51 | 62.50 |
| Prostate | 0.00 | 0.00 | 0.00 | 3.00 | 16.67 | 0.00 |
| Rectum | 6.96 | 1.27 | 4.12 | 1.23 | 40.65 | 167.28 |
| Salivary Gland | 0.34 | 0.53 | 0.44 | 7.31 | 6.84 | 2.98 |
| Clontech | | | | | | |
| Skeletal Muscle | 176.88 | 0.41 | 88.65 | 1.26 | 39.68 | 3517.66 |
| Clontech | | | | | | |
| Skin | 95.17 | 147.16 | 121.17 | 1.21 | 41.32 | 5006.82 |
| Small Intestine | 0.35 | 1.31 | 0.83 | 0.98 | 51.07 | 42.39 |
| Clontech | | | | | | |
| Spleen | 105.73 | 80.76 | 93.25 | 4.92 | 10.16 | 947.61 |
| Stomach | 0.56 | 3.73 | 2.15 | 2.73 | 18.32 | 39.29 |
| Testis Clontech | 0.79 | 0.78 | 0.79 | 0.57 | 87.87 | 68.98 |
| Thymus Clontech | 22.00 | 22.48 | 22.24 | 9.89 | 5.06 | 112.44 |
| Thyroid | 0.65 | 0.48 | 0.57 | 2.77 | 18.05 | 10.20 |
| Trachea Clontech | 1.20 | 0.00 | 0.60 | 9.71 | 5.15 | 3.09 |
| Urinary Bladder | 5.59 | 8.67 | 7.13 | 5.47 | 9.14 | 65.17 |
| Uterus | 19.26 | 27.10 | 23.18 | 5.34 | 9.36 | 217.04 |

| | | | copies of | | |
|---|---|---|---|---|---|
| | Reg | | mRNA | | |
| | number | Mean | detected/50 ng | | Fold Change in |
| Sample | (GSK | GOI | total | | Disease |
| sbg236015LIPASE | identifier) | copies | RNA | Sample | Population |
| colon normal GW98-167 | 21941 | 58.7 | 117.40 | colon normal | |
| colon tumor GW98-166 | 21940 | 300.92 | 601.84 | colon tumor | 5.126405451 |
| colon normal GW98-178 | 22080 | 8.78 | 17.56 | colon normal | |
| colon tumor GW98-177 | 22060 | 23.74 | 47.48 | colon tumor | 2.703872437 |
| colon normal GW98-561 | 23514 | 27.1 | 54.20 | colon normal | |
| colon tumor GW98-560 | 23513 | 39.16 | 78.32 | colon tumor | 1.44501845 |
| colon normal GW98-894 | 24691 | 10.15 | 20.30 | colon normal | |
| colon tumor GW98-893 | 24690 | 144.58 | 289.16 | colon tumor | 14.24433498 |
| lung normal GW98-3 | 20742 | 165.8 | 331.60 | lung normal | |
| lung tumor GW98-2 | 20741 | 80.9 | 161.80 | lung tumor | −2.049443758 |
| lung normal GW97-179 | 20677 | 37.81 | 75.62 | lung normal | |
| lung tumor GW97-178 | 20676 | 109.72 | 219.44 | lung tumor | 2.90187781 |
| lung normal GW98-165 | 21922 | 150.06 | 300.12 | lung normal | |
| lung tumor GW98-164 | 21921 | 169.73 | 339.46 | lung tumor | 1.131080901 |
| lung normal GW98-282 | 22584 | 489.42 | 978.84 | lung normal | |
| lung tumor GW98-281 | 22583 | 188.22 | 376.44 | lung tumor | −2.600255021 |
| breast normal GW00-392 | 28750 | 44.86 | 44.86 | breast | |
| | | | | normal | |
| breast tumor GW00-391 | 28746 | 46.35 | 92.70 | breast tumor | 2.06642889 |
| breast normal GW00-413 | 28798 | 16.35 | 16.35 | breast | |
| | | | | normal | |
| breast tumor GW00-412 | 28797 | 55.98 | 111.96 | breast tumor | 6.847706422 |
| breast normal GW00- | 27592-95 | 3.84 | 3.84 | breast | |
| 235:238 | | | | normal | |
| breast tumor GW00- | 27588-91 | 35.8 | 35.80 | breast tumor | 9.322916667 |
| 231:234 | | | | | |
| breast normal GW98-621 | 23656 | 12.14 | 24.28 | breast | |
| | | | | normal | |
| breast tumor GW98-620 | 23655 | 44.85 | 89.70 | breast tumor | 3.694398682 |
| brain normal BB99-542 | 25507 | 26.03 | 52.06 | brain normal | |
| brain normal BB99-406 | 25509 | 14.78 | 29.56 | brain normal | |
| brain normal BB99-904 | 25546 | 3.39 | 6.78 | brain normal | |
| brain stage 5 ALZ BB99- | 25502 | 35.71 | 71.42 | brain stage 5 | 2.423755656 |
| 874 | | | | ALZ | |
| brain stage 5 ALZ BB99- | 25503 | 9.11 | 18.22 | brain stage 5 | −1.617270399 |
| 887 | | | | ALZ | |
| brain stage 5 ALZ BB99- | 25504 | 8.18 | 16.36 | brain stage 5 | −1.801140994 |
| 862 | | | | ALZ | |
| brain stage 5 ALZ BB99- | 25542 | 46.37 | 92.74 | brain stage 5 | 3.147285068 |
| 927 | | | | ALZ | |
| CT lung KC | normal | 80.77 | 161.54 | CT lung | |
| lung 26 KC | normal | 233.65 | 233.65 | lung 26 | |
| lung 27 KC | normal | 75.27 | 75.27 | lung 27 | |
| lung 24 KC | COPD | 68.64 | 68.64 | lung 24 | −1.876821096 |
| lung 28 KC | COPD | 94.1 | 94.10 | lung 28 | −1.369022317 |
| lung 23 KC | COPD | 88.48 | 88.48 | lung 23 | −1.455978752 |
| lung 25 KC | normal | 44.84 | 44.84 | lung 25 | |
| asthmatic lung ODO3112 | 29321 | 111.42 | 111.42 | asthmatic | −1.156210734 |
| | | | | lung | |
| asthmatic lung ODO3433 | 29323 | 566.5 | 1133.00 | asthmatic | 8.794876771 |
| | | | | lung | |
| asthmatic lung ODO3397 | 29322 | 262.77 | 525.54 | asthmatic | 4.079487677 |
| | | | | lung | |
| asthmatic lung ODO4928 | 29325 | 367.52 | 735.04 | asthmatic | 5.70572482 |
| | | | | lung | |
| endo cells KC | control | 3.23 | 3.23 | endo cells | |
| endo VEGF KC | | 3.41 | 3.41 | endo VEGF | 1.055727554 |
| endo bFGF KC | | 0 | 0.00 | endo bFGF | −3.23 |
| heart Clontech | normal | 0 | 0.00 | heart | |
| heart (T-1) ischemic | 29417 | 35.96 | 71.92 | heart T-1 | 71.92 |
| heart (T-14) non- | 29422 | 18.72 | 37.44 | heart T-14 | 37.44 |
| obstructive DCM | | | | | |
| heart (T-3399) DCM | 29426 | 37.97 | 75.94 | heart T-3399 | 75.94 |
| adenoid GW99-269 | 26162 | 14.17 | 28.34 | adenoid | |
| tonsil GW98-280 | 22582 | 51.21 | 102.42 | tonsil | |
| T cells PC00314 | 28453 | 111.1 | 222.20 | T cells | |
| PBMNC KC | | 162.01 | 162.01 | PBMNC | |
| monocyte KC | | 90.49 | 180.98 | monocyte | |
| B cells PC00665 | 28455 | 109.71 | 219.42 | B cells | |
| dendritic cells 28441 | | 2.44 | 4.88 | dendritic | |
| | | | | cells | |
| neutrophils | 28440 | 1110.91 | 1110.91 | neutrophils | |
| eosinophils | 28446 | 835.72 | 1671.44 | eosinophils | |
| BM unstim KC | | 181.05 | 181.05 | BM unstim | |
| BM stim KC | | 93.96 | 93.96 | BM stim | −1.92688378 |
| osteo dif KC | | 0 | 0.00 | osteo dif | |
| osteo undif KC | | 0.72 | 0.72 | osteo undif | 0.72 |
| chondrocytes | | 2.03 | 5.08 | chondrocytes | |
| OA Synovium IP12/01 | 29462 | 27.82 | 27.82 | OA | |
| | | | | Synovium | |
| OA Synovium NP10/01 | 29461 | 84.94 | 169.88 | OA | |
| | | | | Synovium | |
| OA Synovium NP57/00 | 28464 | 46.58 | 93.16 | OA | |
| | | | | Synovium | |
| RA Synovium NP03/01 | 28466 | 248.24 | 496.48 | RA | |
| | | | | Synovium | |
| RA Synovium NP71/00 | 28467 | 148.32 | 296.64 | RA | |
| | | | | Synovium | |
| RA Synovium NP45/00 | 28475 | 260.28 | 520.56 | RA | |
| | | | | Synovium | |
| OA bone (biobank) | 29217 | 10.27 | 10.27 | OA bone | |
| | | | | (biobank) | |
| OA bone Sample 1 | J. Emory | 17.32 | 34.64 | OA bone | |
| OA bone Sample 2 | J. Emory | 657.01 | 1314.02 | OA bone | |
| Cartilage (pool) | Normal | 59.17 | 118.34 | Cartilage | |
| | | | | (pool) | |
| Cartilage (pool) | OA | 23.33 | 46.66 | Cartilage | −2.53621946 |
| | | | | (pool) | |
| PBL unifected | 28441 | 23.51 | 47.02 | PBL | |
| | | | | unifected | |
| PBL HIV IIIB | 28442 | 5.86 | 11.72 | PBL HIV | −4.011945392 |
| | | | | IIIB | |
| MRC5 uninfected | 29158 | 3.79 | 7.58 | MRC5 | |
| (100%) | | | | uninfected | |
| | | | | (100%) | |
| MRC5 HSV strain F | 29178 | 80.19 | 160.38 | MRC5 HSV | 21.15831135 |
| | | | | strain F | |
| W12 cells | 29179 | 95.42 | 190.84 | W12 cells | |
| Keratinocytes | 29180 | 16.18 | 32.36 | Keratinocytes | |

Gene Name sbg236015LIPASE

| | Fold Change in Disease |
|---|---|
| | Population Relative to |
| Disease tissues | Normal |
| colon tumor | 5.13 |
| colon tumor | 2.70 |
| colon tumor | 1.45 |
| colon tumor | 14.24 |
| lung tumor | −2.05 |
| lung tumor | 2.90 |
| lung tumor | 1.13 |
| lung tumor | −2.60 |
| breast tumor | 2.07 |
| breast tumor | 6.85 |
| breast tumor | 9.32 |
| breast tumor | 3.69 |
| brain stage 5 ALZ | 2.42 |
| brain stage 5 ALZ | −1.62 |
| brain stage 5 ALZ | −1.80 |
| brain stage 5 ALZ | 3.15 |
| lung 24 | −1.88 |
| lung 28 | −1.37 |
| lung 23 | −1.46 |
| asthmatic lung | −1.16 |
| asthmatic lung | 8.79 |
| asthmatic lung | 4.08 |
| asthmatic lung | 5.71 |
| endo VEGF | 1.06 |
| endo bFGF | −3.23 |
| heart T-1 | 71.92 |
| heart T-14 | 37.44 |
| heart T-3399 | 75.94 |
| BM stim | −1.93 |
| osteo undif | 0.72 |
| Cartilage (pool) | −2.54 |
| PBL HIV IIIB | −4.01 |
| MRC5 HSV strain F | 21.16 |

Gene Name sbg417005LAMININ

Expression in adenoid, tonsil and B-cells with corroborating expression in RA/OA samples and asthmatic lung (1/4) suggests involvement in these diseases. Strong expression in brain with overexpression in Alzheimer's disease indicates a role in AD. Down regulation in HSV infected cells suggests potential host cell factor. Expression in colon and lung normal/tumor pairs without corroborating expression in normal tissues suggests immune cell infiltrates.

| | | | | | | copies |
|---|---|---|---|---|---|---|
| | | | | | | of |
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg417005LAMININ | (sample 1) | (sample 2) | Copies | (ng) | (ng) | RNA |
| Subcutaneous | 60.2785303 | 73.59679955 | 66.94 | 3.06 | 16.34 | 1093.75 |
| Adipocytes Zenbio | | | | | | |
| Subcutaneous Adipose | 3.032572965 | 1.985862153 | 2.51 | 0.96 | 52.36 | 131.37 |
| Zenbio | | | | | | |
| Adrenal Gland | 0.965703497 | 0.965703497 | 0.97 | 0.61 | 81.97 | 79.16 |
| Clontech | | | | | | |
| Whole Brain Clontech | 4131.557992 | 6997.879078 | 5564.72 | 7.24 | 6.91 | 38430.38 |
| Fetal Brain Clontech | 0.965703497 | 3.268211325 | 2.12 | 0.48 | 103.95 | 220.06 |
| Cerebellum Clontech | 3.301057867 | 17.3966665 | 10.35 | 2.17 | 23.04 | 238.45 |
| Cervix | 5.920484049 | 7.517891571 | 6.72 | 2.42 | 20.66 | 138.83 |
| Colon | 35.48962684 | 22.53180605 | 29.01 | 2.71 | 18.45 | 535.25 |
| Endometrium | 11.59757492 | 0.965703497 | 6.28 | 0.73 | 68.21 | 428.49 |
| Esophagus | 7.098528857 | 3.523216475 | 5.31 | 1.37 | 36.50 | 193.83 |
| Heart Clontech | 0.965703497 | 5.368977287 | 3.17 | 1.32 | 37.88 | 119.98 |
| Hypothalamus | 0.965703497 | 0.965703497 | 0.97 | 0.32 | 155.28 | 149.95 |
| Ileum | 30.81006847 | 14.15032296 | 22.48 | 2.58 | 19.38 | 435.66 |
| Jejunum | 44.08994058 | 30.29386314 | 37.19 | 6.60 | 7.58 | 281.76 |
| Kidney | 9.424973981 | 15.68529125 | 12.56 | 2.12 | 23.58 | 296.11 |
| Liver | 3.742288161 | 0.965703497 | 2.35 | 1.50 | 33.33 | 78.47 |
| Fetal Liver Clontech | 94.45949484 | 93.8962252 | 94.18 | 10.40 | 4.81 | 452.78 |
| Lung | 13.84782444 | 19.95367566 | 16.90 | 2.57 | 19.46 | 328.81 |
| Mammary Gland | 107.7956161 | 95.02632495 | 101.41 | 13.00 | 3.85 | 390.04 |
| Clontech | | | | | | |
| Myometrium | 12.50117866 | 14.93742804 | 13.72 | 2.34 | 21.37 | 293.15 |
| Omentum | 13.998213 | 22.03816357 | 18.02 | 3.94 | 12.69 | 228.66 |
| Ovary | 0.965703497 | 0.965703497 | 0.97 | 4.34 | 11.52 | 11.13 |
| Pancreas | 2.254750425 | 0.965703497 | 1.61 | 0.81 | 61.80 | 99.52 |
| Head of Pancreas | 0.965703497 | 0.965703497 | 0.97 | 1.57 | 31.85 | 30.75 |
| Parotid Gland | 25.8930892 | 14.85668173 | 20.37 | 5.48 | 9.12 | 185.90 |
| Placenta Clontech | 83.84029668 | 95.02632495 | 89.43 | 5.26 | 9.51 | 850.13 |
| Prostate | 8.047386733 | 15.18245262 | 11.61 | 3.00 | 16.67 | 193.58 |
| Rectum | 10.53572882 | 20.06385011 | 15.30 | 1.23 | 40.65 | 621.94 |
| Salivary Gland | 62.43024331 | 57.19623352 | 59.81 | 7.31 | 6.84 | 409.12 |
| Clontech | | | | | | |
| Skeletal Muscle | 1.376746214 | 0.965703497 | 1.17 | 1.26 | 39.68 | 46.48 |
| Clontech | | | | | | |
| Skin | 0.965703497 | 0.965703497 | 0.97 | 1.21 | 41.32 | 39.91 |
| Small Intestine | 0.965703497 | 0.965703497 | 0.97 | 0.98 | 51.07 | 49.32 |
| Clontech | | | | | | |
| Spleen | 0.965703497 | 5.740147492 | 3.35 | 4.92 | 10.16 | 34.07 |
| Stomach | 0.965703497 | 0.965703497 | 0.97 | 2.73 | 18.32 | 17.69 |
| Testis Clontech | 0.965703497 | 0.965703497 | 0.97 | 0.57 | 87.87 | 84.86 |
| Thymus Clontech | 258.7386545 | 207.7169358 | 233.23 | 9.89 | 5.06 | 1179.11 |
| Thyroid | 12.56849785 | 19.09489343 | 15.83 | 2.77 | 18.05 | 285.77 |
| Trachea Clontech | 24.35330878 | 31.87047641 | 28.11 | 9.71 | 5.15 | 144.76 |
| Urinary Bladder | 51.81831091 | 57.53035871 | 54.67 | 5.47 | 9.14 | 499.77 |
| Uterus | 13.12099559 | 14.61718971 | 13.87 | 5.34 | 9.36 | 129.86 |

| | | | copies of | | |
|---|---|---|---|---|---|
| | | | mRNA | | |
| | Reg | | detected/ | | |
| | number | | 50 ng | | Fold Change |
| Sample | (GSK | Mean GOI | total | | in Disease |
| sbg417005LAMININ | identifier) | copies | RNA | Sample | Population |
| colon normal GW98-167 | 21941 | 15446.92728 | 30893.85 | colon normal | |
| colon tumor GW98-166 | 21940 | 23910.90415 | 47821.81 | colon tumor | 1.547939193 |
| colon normal GW98-178 | 22080 | 14621.97321 | 29243.95 | colon normal | |
| colon tumor GW98-177 | 22060 | 2058.30396 | 4116.61 | colon tumor | −7.10389403 |
| colon normal GW98-561 | 23514 | 5590.900474 | 11181.80 | colon normal | |
| colon tumor GW98-560 | 23513 | 12318.10362 | 24636.21 | colon tumor | 2.203241442 |
| colon normal GW98-894 | 24691 | 4478.692403 | 8957.38 | colon normal | |
| colon tumor GW98-893 | 24690 | 7546.100944 | 15092.20 | colon tumor | 1.684889308 |
| lung normal GW98-3 | 20742 | 23910.90415 | 47821.81 | lung normal | |
| lung tumor GW98-2 | 20741 | 35021.23317 | 70042.47 | lung tumor | 1.464655328 |
| lung normal GW97-179 | 20677 | 23341.61421 | 46683.23 | lung normal | |
| lung tumor GW97-178 | 20676 | 24103.90252 | 48207.81 | lung tumor | 1.032657909 |
| lung normal GW98-165 | 21922 | 18374.41273 | 36748.83 | lung normal | |
| lung tumor GW98-164 | 21921 | 34735.19726 | 69470.39 | lung tumor | 1.890411289 |
| lung normal GW98-282 | 22584 | 3002.298467 | 6004.60 | lung normal | |
| lung tumor GW98-281 | 22583 | 3519.560955 | 7039.12 | lung tumor | 1.172288829 |
| breast normal GW00-392 | 28750 | 5978.671937 | 5978.67 | breast | |
| | | | | normal | |
| breast tumor GW00-391 | 28746 | 5674.721186 | 11349.44 | breast tumor | 1.898321649 |
| breast normal GW00-413 | 28798 | 1523.643258 | 1523.64 | breast | |
| | | | | normal | |
| breast tumor GW00-412 | 28797 | 956.0902914 | 1912.18 | breast tumor | 1.255005444 |
| breast normal GW00- | 27592-95 | 760.6128764 | 760.61 | breast | |
| 235:238 | | | | normal | |
| breast tumor GW00- | 27588-91 | 4192.50003 | 4192.50 | breast tumor | 5.51200244 |
| 231:234 | | | | | |
| breast normal GW98-621 | 23656 | 5674.721186 | 11349.44 | breast | |
| | | | | normal | |
| breast tumor GW98-620 | 23655 | 8017.202071 | 16034.40 | breast tumor | 1.412792243 |
| brain normal BB99-542 | 25507 | 791.7818289 | 1583.56 | brain normal | |
| brain normal BB99-406 | 25509 | 524.990001 | 1049.98 | brain normal | |
| brain normal BB99-904 | 25546 | 396.8655236 | 793.73 | brain normal | |
| brain stage 5 ALZ BB99- | 25502 | 3203.498645 | 6407.00 | brain stage 5 | 5.608243725 |
| 874 | | | | ALZ | |
| brain stage 5 ALZ BB99- | 25503 | 3925.505917 | 7851.01 | brain stage 5 | 6.872234505 |
| 887 | | | | ALZ | |
| brain stage 5 ALZ BB99- | 25504 | 1502.651942 | 3005.30 | brain stage 5 | 2.630635833 |
| 862 | | | | ALZ | |
| brain stage 5 ALZ BB99- | 25542 | 1555.711325 | 3111.42 | brain stage 5 | 2.723524884 |
| 927 | | | | ALZ | |
| CT lung KC | normal | 3730.249874 | 7460.50 | CT lung | |
| lung 26 KC | normal | 286.3143862 | 286.31 | lung 26 | |
| lung 27 KC | normal | 72.30560941 | 72.31 | lung 27 | |
| lung 24 KC | COPD | 28.47771374 | 28.48 | lung 24 | −69.25877363 |
| lung 28 KC | COPD | 66.98006875 | 66.98 | lung 28 | −29.44654382 |
| lung 23 KC | COPD | 57.53035871 | 57.53 | lung 23 | −34.28331708 |
| lung 25 KC | COPD | 70.20637402 | 70.21 | lung 25 | |
| asthmatic lung | 29321 | 2304.915385 | 2304.92 | asthmatic | 1.168624722 |
| ODO3112 | | | | lung | |
| asthmatic lung | 29323 | 3112.377018 | 6224.75 | asthmatic | 3.156038395 |
| ODO3433 | | | | lung | |
| asthmatic lung | 29322 | 21892.2071 | 43784.41 | asthmatic | 22.19931768 |
| ODO3397 | | | | lung | |
| asthmatic lung | 29325 | 5268.438364 | 10536.88 | asthmatic | 5.34234563 |
| ODO4928 | | | | lung | |
| endo cells KC | control | 396.8655236 | 396.87 | endo cells | |
| endo VEGF KC | | 157.1987188 | 157.20 | endo VEGF | −2.524610421 |
| endo bFGF KC | | 518.1542863 | 518.15 | endo bFGF | 1.305616778 |
| heart Clontech | normal | 1865.302957 | 3730.61 | heart | |
| heart (T-1) ischemic | 29417 | 3757.505456 | 7515.01 | heart T-1 | 2.014421005 |
| heart (T-14) non- | 29422 | 1633.333543 | 3266.67 | heart T-14 | −1.142022072 |
| obstructive DCM | | | | | |
| heart (T-3399) DCM | 29426 | 2938.226492 | 5876.45 | heart T-3399 | 1.575200683 |
| adenoid GW99-269 | 26162 | 1238.725105 | 2477.45 | adenoid | |
| tonsil GW98-280 | 22582 | 2288.625236 | 4577.25 | tonsil | |
| T cells PC00314 | 28453 | 61.34444995 | 122.69 | T cells | |
| PBMNC KC | | 5.341492957 | 5.34 | PBMNC | |
| monocyte KC | | 3.576686692 | 7.15 | monocyte | |
| B cells PC00665 | 28455 | 716.2601536 | 1432.52 | B cells | |
| dendritic cells 28441 | | 32.23243314 | 64.46 | dendritic | |
| | | | | cells | |
| neutrophils | 28440 | 32.9693996 | 32.97 | neutrophils | |
| eosinophils | 28446 | 1.444144312 | 2.89 | eosinophils | |
| BM unstim KC | | 5.951115795 | 5.95 | BM unstim | |
| BM stim KC | | 11.72233235 | 11.72 | BM stim | 1.969770503 |
| osteo dif KC | | 10.20495465 | 10.20 | osteo dif | |
| osteo undif KC | | 8.526098078 | 8.53 | osteo undif | −1.196907959 |
| chondrocytes | | 14621.97321 | 36554.93 | chondrocytes | |
| OA Synovium IP12/01 | 29462 | 5549.480142 | 5549.48 | OA | |
| | | | | Synovium | |
| OA Synovium NP10/01 | 29461 | 3545.197127 | 7090.39 | OA | |
| | | | | Synovium | |
| OA Synovium NP57/00 | 28464 | 4223.325454 | 8446.65 | OA | |
| | | | | Synovium | |
| RA Synovium NP03/01 | 28466 | 1221.845309 | 2443.69 | RA | |
| | | | | Synovium | |
| RA Synovium NP71/00 | 28467 | 4892.67872 | 9785.36 | RA | |
| | | | | Synovium | |
| RA Synovium NP45/00 | 28475 | 1080.396739 | 2160.79 | RA | |
| | | | | Synovium | |
| OA bone (biobank) | 29217 | 995.7612933 | 995.76 | OA bone | |
| | | | | (biobank) | |
| OA bone Sample 1 | J. Emory | 982.3483914 | 1964.70 | OA bone | |
| OA bone Sample 2 | J. Emory | 472.8535333 | 945.71 | OA bone | |
| Cartilage (pool) | Normal | 1213.496434 | 2426.99 | Cartilage | |
| | | | | (pool) | |
| Cartilage (pool) | OA | 697.4302173 | 1394.86 | Cartilage | −1.73995391 |
| | | | | (pool) | |
| PBL unifected | 28441 | 161.1142664 | 322.23 | PBL | |
| | | | | unifected | |
| PBL HIV IIIB | 28442 | 191.5686557 | 383.14 | PBL HIV | 1.189023542 |
| | | | | IIIB | |
| MRC5 uninfected | 29158 | 5934.220593 | 11868.44 | MRC5 | |
| (100%) | | | | uninfected | |
| | | | | (100%) | |
| MRC5 HSV strain F | 29178 | 50.63206269 | 101.26 | MRC5 HSV | −117.2028213 |
| | | | | strain F | |
| W12 cells | 29179 | 13843.2955 | 27686.59 | W12 cells | |
| Keratinocytes | 29180 | 11849.9156 | 23699.83 | Keratinocytes | |

Gene Name sbg417005LAMININ

| | Fold Change in Disease |
|---|---|
| | Population Relative to |
| Disease tissues | Normal |
| colon tumor | 1.55 |
| colon tumor | −7.10 |
| colon tumor | 2.20 |
| colon tumor | 1.68 |
| lung tumor | 1.46 |
| lung tumor | 1.03 |
| lung tumor | 1.89 |
| lung tumor | 1.17 |
| breast tumor | 1.90 |
| breast tumor | 1.26 |
| breast tumor | 5.51 |
| breast tumor | 1.41 |
| brain stage 5 ALZ | 5.61 |
| brain stage 5 ALZ | 6.87 |
| brain stage 5 ALZ | 2.63 |
| brain stage 5 ALZ | 2.72 |
| lung 24 | −69.26 |
| lung 28 | −29.45 |
| lung 23 | −34.28 |
| asthmatic lung | 1.17 |
| asthmatic lung | 3.16 |
| asthmatic lung | 22.20 |
| asthmatic lung | 5.34 |
| endo VEGF | −2.52 |
| endo bFGF | 1.31 |
| heart T-1 | 2.01 |
| heart T-14 | −1.14 |
| heart T-3399 | 1.58 |
| BM stim | 1.97 |
| osteo undif | −1.20 |
| Cartilage (pool) | −1.74 |
| PBL HIV IIIB | 1.19 |
| MRC5 HSV strain F | −117.20 |

Gene Name sbg425649KINASEa

Strongly expressed in neutrophils and eosinophils suggesting function in immune system such as involvement in allergic reactions and anti-infective. Lower expression in T-cells. Expression in 2/3 OA bone samples indicate a role in OA. Strongly expressed in rectum and skeletal muscle, unknown function.

| | | | | | | copies of |
|---|---|---|---|---|---|---|
| | | | | | | mRNA |
| | | | | | 50 | detected/ |
| | Mean GOI | Mean GOI | Average | 18S | ng/18S | 50 ng |
| Sample | copies | copies | GOI | rRNA | rRNA | total |
| sbg425649KINASEa | (sample 1) | (sample 2) | Copies | (ng) | (ng) | RNA |
| Subcutaneous | 0.00 | 0.03 | 0.02 | 3.06 | 16.34 | 0.25 |
| Adipocytes Zenbio | | | | | | |
| Subcutaneous Adipose | 0.00 | 0.00 | 0.00 | 0.96 | 52.36 | 0.00 |
| Zenbio | | | | | | |
| Adrenal Gland Clontech | 0.23 | 0.00 | 0.12 | 0.61 | 81.97 | 9.43 |
| Whole Brain Clontech | 163.64 | 47.63 | 105.64 | 7.24 | 6.91 | 729.52 |
| Fetal Brain Clontech | 0.47 | 0.00 | 0.24 | 0.48 | 103.95 | 24.43 |
| Cerebellum Clontech | 0.00 | 0.00 | 0.00 | 2.17 | 23.04 | 0.00 |
| Cervix | 5.54 | 0.00 | 2.77 | 2.42 | 20.66 | 57.23 |
| Colon | 0.70 | 0.00 | 0.35 | 2.71 | 18.45 | 6.46 |
| Endometrium | 0.33 | 0.06 | 0.20 | 0.73 | 68.21 | 13.30 |
| Esophagus | 0.35 | 0.47 | 0.41 | 1.37 | 36.50 | 14.96 |
| Heart Clontech | 0.00 | 0.00 | 0.00 | 1.32 | 37.88 | 0.00 |
| Hypothalamus | 0.00 | 0.00 | 0.00 | 0.32 | 155.28 | 0.00 |
| Ileum | 0.00 | 4.49 | 2.25 | 2.58 | 19.38 | 43.51 |
| Jejunum | 0.29 | 0.73 | 0.51 | 6.60 | 7.58 | 3.86 |
| Kidney | 0.00 | 0.00 | 0.00 | 2.12 | 23.58 | 0.00 |
| Liver | 10.48 | 5.64 | 8.06 | 1.50 | 33.33 | 268.67 |
| Fetal Liver Clontech | 8.56 | 0.00 | 4.28 | 10.40 | 4.81 | 20.58 |
| Lung | 0.00 | 0.00 | 0.00 | 2.57 | 19.46 | 0.00 |
| Mammary Gland | 0.00 | 0.00 | 0.00 | 13.00 | 3.85 | 0.00 |
| Clontech | | | | | | |
| Myometrium | 8.61 | 5.00 | 6.81 | 2.34 | 21.37 | 145.41 |
| Omentum | 0.23 | 10.99 | 5.61 | 3.94 | 12.69 | 71.19 |
| Ovary | 4.48 | 4.62 | 4.55 | 4.34 | 11.52 | 52.42 |
| Pancreas | 0.27 | 0.00 | 0.14 | 0.81 | 61.80 | 8.34 |
| Head of Pancreas | 0.11 | 0.04 | 0.08 | 1.57 | 31.85 | 2.39 |
| Parotid Gland | 0.69 | 4.51 | 2.60 | 5.48 | 9.12 | 23.72 |
| Placenta Clontech | 10.58 | 0.14 | 5.36 | 5.26 | 9.51 | 50.95 |
| Prostate | 9.74 | 6.18 | 7.96 | 3.00 | 16.67 | 132.67 |
| Rectum | 225.51 | 76.99 | 151.25 | 1.23 | 40.65 | 6148.37 |
| Salivary Gland | 60.93 | 67.22 | 64.08 | 7.31 | 6.84 | 438.27 |
| Clontech | | | | | | |
| Skeletal Muscle | 749.28 | 29.78 | 389.53 | 1.26 | 39.68 | 15457.54 |
| Clontech | | | | | | |
| Skin | 0.00 | 4.46 | 2.23 | 1.21 | 41.32 | 92.15 |
| Small Intestine | 0.73 | 0.00 | 0.37 | 0.98 | 51.07 | 18.64 |
| Clontech | | | | | | |
| Spleen | 4.10 | 8.60 | 6.35 | 4.92 | 10.16 | 64.53 |
| Stomach | 4.24 | 19.28 | 11.76 | 2.73 | 18.32 | 215.38 |
| Testis Clontech | 10.11 | 6.34 | 8.23 | 0.57 | 87.87 | 722.76 |
| Thymus Clontech | 2.79 | 5.35 | 4.07 | 9.89 | 5.06 | 20.58 |
| Thyroid | 0.00 | 0.06 | 0.03 | 2.77 | 18.05 | 0.54 |
| Trachea Clontech | 5.24 | 14.14 | 9.69 | 9.71 | 5.15 | 49.90 |
| Urinary Bladder | 0.09 | 0.00 | 0.05 | 5.47 | 9.14 | 0.41 |
| Uterus | 27.26 | 7.61 | 17.44 | 5.34 | 9.36 | 163.25 |

| | | | copies of | | |
|---|---|---|---|---|---|
| | | | mRNA | | |
| | Reg | | detected/ | | |
| | number | Mean | 50 ng | | Fold Change |
| Sample | (GSK | GOI | total | | in Disease |
| sbg425649KINASEa | identifier) | copies | RNA | Sample | Population |
| colon normal GW98-167 | 21941 | 11.11 | 22.22 | colon normal | |
| colon tumor GW98-166 | 21940 | 7.3 | 14.60 | colon tumor | −1.521917808 |
| colon normal GW98-178 | 22080 | 0 | 0.00 | colon normal | |
| colon tumor GW98-177 | 22060 | 2.57 | 5.14 | colon tumor | 5.14 |
| colon normal GW98-561 | 23514 | 0 | 0.00 | colon normal | |
| colon tumor GW98-560 | 23513 | 0 | 0.00 | colon tumor | 0 |
| colon normal GW98-894 | 24691 | 2.71 | 5.42 | colon normal | |
| colon tumor GW98-893 | 24690 | 8.51 | 17.02 | colon tumor | 3.140221402 |
| lung normal GW98-3 | 20742 | 1.78 | 3.56 | lung normal | |
| lung tumor GW98-2 | 20741 | 0 | 0.00 | lung tumor | −3.56 |
| lung normal GW97-179 | 20677 | 3.18 | 6.36 | lung normal | |
| lung tumor GW97-178 | 20676 | 2.64 | 5.28 | lung tumor | −1.204545455 |
| lung normal GW98-165 | 21922 | 6.46 | 12.92 | lung normal | |
| lung tumor GW98-164 | 21921 | 19.99 | 39.98 | lung tumor | 3.094427245 |
| lung normal GW98-282 | 22584 | 31.56 | 63.12 | lung normal | |
| lung tumor GW98-281 | 22583 | 7.47 | 14.94 | lung tumor | −4.224899598 |
| breast normal GW00-392 | 28750 | 5.68 | 5.68 | breast | |
| | | | | normal | |
| breast tumor GW00-391 | 28746 | 2.87 | 5.74 | breast tumor | 1.01056338 |
| breast normal GW00-413 | 28798 | 1.66 | 1.66 | breast | |
| | | | | normal | |
| breast tumor GW00-412 | 28797 | 1.99 | 3.98 | breast tumor | 2.397590361 |
| breast normal GW00- | 27592-95 | 0 | 0.00 | breast | |
| 235:238 | | | | normal | |
| breast tumor GW00- | 27588-91 | 2.19 | 2.19 | breast tumor | 2.19 |
| 231:234 | | | | | |
| breast normal GW98-621 | 23656 | 4.72 | 9.44 | breast | |
| | | | | normal | |
| breast tumor GW98-620 | 23655 | 0 | 0.00 | breast tumor | −9.44 |
| brain normal BB99-542 | 25507 | 28.9 | 57.80 | brain normal | |
| brain normal BB99-406 | 25509 | 24.84 | 49.68 | brain normal | |
| brain normal BB99-904 | 25546 | 6.92 | 13.84 | brain normal | |
| brain stage 5 ALZBB99- | 25502 | 23.65 | 47.30 | brain stage 5 | 1.169634026 |
| 874 | | | | ALZ | |
| brain stage 5 ALZ BB99- | 25503 | 28.68 | 57.36 | brain stage 5 | 1.418397626 |
| 887 | | | | ALZ | |
| brain stage 5 ALZ BB99- | 25504 | 18.18 | 36.36 | brain stages 5 | −1.112211221 |
| 862 | | | | ALZ | |
| brain stage 5 ALZ BB99- | 25542 | 14.18 | 28.36 | brain stage 5 | −1.425952045 |
| 927 | | | | ALZ | |
| CT lung KC | normal | 29.45 | 58.90 | CT lung | |
| lung 26 KC | normal | 2.47 | 2.47 | lung 26 | |
| lung 27 KC | normal | 0 | 0.00 | lung 27 | |
| lung 24 KC | COPD | 0 | 0.00 | lung 24 | −15.3425 |
| lung 28 KC | COPD | 0.3 | 0.30 | lung 28 | −51.14166667 |
| lung 23 KC | COPD | 0 | 0.00 | lung 23 | −15.3425 |
| lung 25 KC | COPD | 0 | 0.00 | lung 25 | |
| asthmatic lung | 29321 | 3.24 | 3.24 | asthmatic | −4.735339506 |
| ODO3112 | | | | lung | |
| asthmatic lung | 29323 | 88.32 | 176.64 | asthmatic | 11.51311716 |
| ODO3433 | | | | lung | |
| asthmatic lung | 29322 | 55.65 | 111.30 | asthmatic | 7.254358807 |
| ODO3397 | | | | lung | |
| asthmatic lung | 29325 | 50.64 | 101.28 | asthmatic | 6.601270979 |
| ODO4928 | | | | lung | |
| endo cells KC | control | 0 | 0.00 | endo cells | |
| endo VEGF KC | | 0 | 0.00 | endo VEGF | 0 |
| endo bFGF KC | | 0 | 0.00 | endo bFGF | 0 |
| heart Clontech | normal | 15.26 | 30.52 | heart | |
| heart (T-1 ) ischemic | 29417 | 0 | 0.00 | heart T-1 | −30.52 |
| heart (T-14) non- | 29422 | 3.69 | 7.38 | heart T-14 | −4.135501355 |
| obstructive DCM | | | | | |
| heart (T-3399) DCM | 29426 | 0 | 0.00 | heart T-3399 | −30.52 |
| adenoid GW99-269 | 26162 | 0 | 0.00 | adenoid | |
| tonsil GW98-280 | 22582 | 3.65 | 7.30 | tonsil | |
| T cells PC00314 | 28453 | 167.51 | 335.02 | T cells | |
| PBMNC KC | | 2.5 | 2.50 | PBMNC | |
| monocyte KC | | 2.37 | 4.74 | monocyte | |
| B cells PC00665 | 28455 | 0 | 0.00 | B cells | |
| dendritic cells 28441 | | 0 | 0.00 | dendritic | |
| | | | | cells | |
| neutrophils | 28440 | 1576.76 | 1576.76 | neutrophils | |
| eosinophils | 28446 | 755.1 | 1510.20 | eosinophils | |
| BM unstim KC | | 14.87 | 14.87 | BM unstim | |
| BM stim KC | | 45.45 | 45.45 | BM stim | 3.056489576 |
| osteo dif KC | | 0 | 0.00 | osteo dif | |
| osteo undif KC | | 0 | 0.00 | osteo undif | 0 |
| chondrocytes | | 7.48 | 18.70 | chondrocytes | |
| OA Synovium IP12/01 | 29462 | 17.79 | 17.79 | OA | |
| | | | | Synovium | |
| OA Synovium NP10/01 | 29461 | 14.09 | 28.18 | OA | |
| | | | | Synovium | |
| OA Synovium NP57/00 | 28464 | 11.97 | 23.94 | OA | |
| | | | | Synovium | |
| RA Synovium NP03/01 | 28466 | 6.84 | 13.68 | RA | |
| | | | | Synovium | |
| RA Synovium NP71/00 | 28467 | 22.88 | 45.76 | RA | |
| | | | | Synovium | |
| RA Synovium NP45/00 | 28475 | 1.64 | 3.28 | RA | |
| | | | | Synovium | |
| OA bone (biobank) | 29217 | 370.22 | 370.22 | OA bone | |
| | | | | (biobank) | |
| OA bone Sample 1 | J. Emory | 3.21 | 6.42 | OA bone | |
| OA bone Sample 2 | J. Emory | 311.65 | 623.30 | OA bone | |
| Cartilage (pool) | Normal | 32.23 | 64.46 | Cartilage | |
| | | | | (pool) | |
| Cartilage (pool) | OA | 2.87 | 5.74 | Cartilage | −11.22996516 |
| | | | | (pool) | |
| PBL unifected | 28441 | 4.18 | 8.36 | PBL | |
| | | | | unifected | |
| PBL HIV IIIB | 28442 | 0 | 0.00 | PBL HIV | −8.36 |
| | | | | IIIB | |
| MRC5 uninfected | 29158 | 4.4 | 8.80 | MRC5 | |
| (100%) | | | | uninfected | |
| | | | | (100%) | |
| MRC5 HSV strain F | 29178 | 11.46 | 22.92 | MRC5 HSV | 2.604545455 |
| | | | | strain F | |
| W12 cells | 29179 | 0 | 0.00 | W12 cells | |
| Keratinocytes | 29180 | 0 | 0.00 | Keratinocytes | |

Gene Name sbg425649KINASEa

| | Fold Change in Disease |
|---|---|
| | Population Relative to |
| Disease tissues | Normal |
| colon tumor | −1.52 |
| colon tumor | 5.14 |
| colon tumor | 0.00 |
| colon tumor | 3.14 |
| lung tumor | −3.56 |
| lung tumor | −1.20 |
| lung tumor | 3.09 |
| lung tumor | −4.22 |
| breast tumor | 1.01 |
| breast tumor | 2.40 |
| breast tumor | 2.19 |
| breast tumor | −9.44 |
| brain stage 5 ALZ | 1.17 |
| brain stage 5 ALZ | 1.42 |
| brain stage 5 ALZ | −1.11 |
| brain stage 5 ALZ | −1.43 |
| lung 24 | −15.34 |
| lung 28 | −51.14 |
| lung 23 | −15.34 |
| asthmatic lung | −4.74 |
| asthmatic lung | 11.51 |
| asthmatic lung | 7.25 |
| asthmatic lung | 6.60 |
| endo VEGF | 0.00 |
| endo bFGF | 0.00 |
| heart T-1 | −30.52 |
| heart T-14 | −4.14 |
| heart T-3399 | −30.52 |
| BM stim | 3.06 |
| osteo undif | 0.00 |
| Cartilage (pool) | −11.23 |
| PBL HIV IIIB | −8.36 |
| MRC5 HSV strain F | 2.60 |

Gene Name sbg419582PROTOCADHERIN

Brain specific expression. No correlation with Alzheimer's disease. Low expression in RA and OA synovium but no corroborating expression in immune cells. Slightly upregulated in heart disease. Overexpressed in lung (1/4) and breast (1/4) tumors.

| | | | | | | copies of |
|---|---|---|---|---|---|---|
| | | | | | | mRNA |
| | | | | | 50 ng/ | detected/ |
| Sample | Mean GOI | Mean GOI | Average | 18S | 18S | 50 ng |
| sbg419582PROTOCA | copies | copies | GOI | rRNA | rRNA | total |
| DHERIN | (sample 1) | (sample 2) | Copies | (ng) | (ng) | RNA |
| Subcutaneous | 18.18 | 23.43 | 20.81 | 3.06 | 16.34 | 339.95 |
| Adipocytes Zenbio | | | | | | |
| Subcutaneous Adipose | 0.11 | 0.33 | 0.22 | 0.96 | 52.36 | 11.52 |
| Zenbio | | | | | | |
| Adrenal Gland Clontech | 1.8 | 1.06 | 1.43 | 0.61 | 81.97 | 117.21 |
| Whole Brain Clontech | 10913.92 | 10314.42 | 10614.17 | 7.24 | 6.91 | 73302.28 |
| Fetal Brain Clontech | 0.31 | 4.68 | 2.50 | 0.48 | 103.95 | 259.36 |
| Cerebellum Clontech | 0.1 | 4.58 | 2.34 | 2.17 | 23.04 | 53.92 |
| Cervix | 0.22 | 1.22 | 0.72 | 2.42 | 20.66 | 14.88 |
| Colon | 0.31 | 13.73 | 7.02 | 2.71 | 18.45 | 129.52 |
| Endometrium | 0.1 | 0.58 | 0.34 | 0.73 | 68.21 | 23.19 |
| Esophagus | 2.21 | 1.96 | 2.09 | 1.37 | 36.50 | 76.09 |
| Heart Clontech | 0.32 | 0 | 0.16 | 1.32 | 37.88 | 6.06 |
| Hypothalamus | 0.15 | 1.2 | 0.68 | 0.32 | 155.28 | 104.81 |
| Ileum | 2.77 | 1.03 | 1.90 | 2.58 | 19.38 | 36.82 |
| Jejunum | 0.26 | 1.18 | 0.72 | 6.60 | 7.58 | 5.45 |
| Kidney | 1.99 | 0.28 | 1.14 | 2.12 | 23.58 | 26.77 |
| Liver | 7.59 | 12.42 | 10.01 | 1.50 | 33.33 | 333.50 |
| Fetal Liver Clontech | 18.75 | 11.04 | 14.90 | 10.40 | 4.81 | 71.61 |
| Lung | 7.19 | 0.71 | 3.95 | 2.57 | 19.46 | 76.85 |
| Mammary Gland | 88.14 | 97.88 | 93.01 | 13.00 | 3.85 | 357.73 |
| Clontech | | | | | | |
| Myometrium | 0.51 | 4.8 | 2.66 | 2.34 | 21.37 | 56.73 |
| Omentum | 7.52 | 2.19 | 4.86 | 3.94 | 12.69 | 61.61 |
| Ovary | 13.46 | 4.84 | 9.15 | 4.34 | 11.52 | 105.41 |
| Pancreas | 0.49 | 1.02 | 0.76 | 0.81 | 61.80 | 46.66 |
| Head of Pancreas | 0.29 | 0.15 | 0.22 | 1.57 | 31.85 | 7.01 |
| Parotid Gland | 6.09 | 6.19 | 6.14 | 5.48 | 9.12 | 56.02 |
| Placenta Clontech | 10.67 | 2.35 | 6.51 | 5.26 | 9.51 | 61.88 |
| Prostate | 2.02 | 3.59 | 2.81 | 3.00 | 16.67 | 46.75 |
| Rectum | 0.54 | 7.25 | 3.90 | 1.23 | 40.65 | 158.33 |
| Salivary Gland | 20.51 | 13.73 | 17.12 | 7.31 | 6.84 | 117.10 |
| Clontech | | | | | | |
| Skeletal Muscle | 1.06 | 0.79 | 0.93 | 1.26 | 39.68 | 36.71 |
| Clontech | | | | | | |
| Skin | 13.09 | 0.6 | 6.85 | 1.21 | 41.32 | 282.85 |
| Small Intestine | 0.11 | 2.47 | 1.29 | 0.98 | 51.07 | 65.88 |
| Clontech | | | | | | |
| Spleen | 1.05 | 11 | 6.03 | 4.92 | 10.16 | 61.23 |
| Stomach | 0.95 | 1.3 | 1.13 | 2.73 | 18.32 | 20.60 |
| Testis Clontech | 2.82 | 3.19 | 3.01 | 0.57 | 87.87 | 264.06 |
| Thymus Clontech | 117.82 | 118.81 | 118.32 | 9.89 | 5.06 | 598.15 |
| Thyroid | 2.34 | 2.29 | 2.32 | 2.77 | 18.05 | 41.79 |
| Trachea Clontech | 8.72 | 9.37 | 9.05 | 9.71 | 5.15 | 46.58 |
| Urinary Bladder | 14.23 | 16.82 | 15.53 | 5.47 | 9.14 | 141.91 |
| Uterus | 1.49 | 27.26 | 14.38 | 5.34 | 9.36 | 134.60 |

| | | | copies of | | |
|---|---|---|---|---|---|
| | Reg | | mRNA | | |
| Sample | number | Mean | detected/50 ng | | Fold Change in |
| sbg419582PROTOCA | (GSK | GOI | total | | Disease |
| DHERIN | identifier) | copies | RNA | Sample | Population |
| colon normal GW98-167 | 21941 | 464.48 | 928.96 | colon normal | |
| colon tumor GW98-166 | 21940 | 84.22 | 168.44 | colon tumor | −5.515079554 |
| colon normal GW98-178 | 22080 | 32.8 | 65.60 | colon normal | |
| colon tumor GW98-177 | 22060 | 44.71 | 89.42 | colon tumor | 1.363109756 |
| colon normal GW98-561 | 23514 | 135.5 | 271.00 | colon normal | |
| colon tumor GW98-560 | 23513 | 78.51 | 157.02 | colon tumor | −1.72589479 |
| colon normal GW98-894 | 24691 | 454.16 | 908.32 | colon normal | |
| colon tumor GW98-893 | 24690 | 51.37 | 102.74 | colon tumor | −8.840957757 |
| lung normal GW98-3 | 20742 | 60.35 | 120.70 | lung normal | |
| lung tumor GW98-2 | 20741 | 101.98 | 203.96 | lung tumor | 1.689809445 |
| lung normal GW97-179 | 20677 | 264 | 528.00 | lung normal | |
| lung tumor GW97-178 | 20676 | 78.49 | 156.98 | lung tumor | −3.363485794 |
| lung normal GW98-165 | 21922 | 88.19 | 176.38 | lung normal | |
| lung tumor GW98-164 | 21921 | 7554.58 | 15109.16 | lung tumor | 85.66254677 |
| lung normal GW98-282 | 22584 | 344.2 | 688.40 | lung normal | |
| lung tumor GW98-281 | 22583 | 45.51 | 91.02 | lung tumor | −7.563172929 |
| breast normal GW00-392 | 28750 | 132.43 | 132.43 | breast | |
| | | | | normal | |
| breast tumor GW00-391 | 28746 | 98.14 | 196.28 | breast tumor | 1.482141509 |
| breast normal GW00-413 | 28798 | 154.37 | 154.37 | breast | |
| | | | | normal | |
| breast tumor GW00-412 | 28797 | 1289.09 | 2578.18 | breast tumor | 16.70130207 |
| breast normal GW00- | 27592-95 | 18.63 | 18.63 | breast | |
| 235:238 | | | | normal | |
| breast tumor GW00- | 27588-91 | 133.52 | 133.52 | breast tumor | 7.166935051 |
| 231:234 | | | | | |
| breast normal GW98-621 | 23656 | 1334.91 | 2669.82 | breast | |
| | | | | normal | |
| breast tumor GW98-620 | 23655 | 212.39 | 424.78 | breast tumor | −6.285182918 |
| brain normal BB99-542 | 25507 | 6816.47 | 13632.94 | brain normal | |
| brain normal BB99-406 | 25509 | 1984.48 | 3968.96 | brain normal | |
| brain normal BB99-904 | 25546 | 2805.82 | 5611.64 | brain normal | |
| brain stage 5 ALZ BB99- | 25502 | 467.59 | 935.18 | brain stage 5 | −8.274178946 |
| 874 | | | | ALZ | |
| brain stage 5 ALZ BB99- | 25503 | 3104.22 | 6208.44 | brain stage 5 | −1.24634315 |
| 887 | | | | ALZ | |
| brain stage 5 ALZ BB99- | 25504 | 1889.81 | 3779.62 | brain stage 5 | −2.047255191 |
| 862 | | | | ALZ | |
| brain stage 5 ALZ BB99- | 25542 | 2902.29 | 5804.58 | brain stage 5 | −1.333058837 |
| 927 | | | | ALZ | |
| CT lung KC | normal | 103.32 | 206.64 | CT lung | |
| lung 26 KC | normal | 1.13 | 1.13 | lung 26 | |
| lung 27 KC | normal | 1.51 | 1.51 | lung 27 | |
| lung 24 KC | COPD | 1.47 | 1.47 | lung 24 | −35.82312925 |
| lung 28 KC | COPD | 0 | 0.00 | lung 28 | −52.66 |
| lung 23 KC | COPD | 1.91 | 1.91 | lung 23 | −27.57068063 |
| lung 25 KC | COPD | 1.36 | 1.36 | lung 25 | |
| asthmatic lung | 29321 | 2.68 | 2.68 | asthmatic | −19.64925373 |
| ODO3112 | | | | lung | |
| asthmatic lung | 29323 | 3.25 | 6.50 | asthmatic | −8.101538462 |
| ODO3433 | | | | lung | |
| asthmatic lung | 29322 | 26.23 | 52.46 | asthmatic | −1.003812429 |
| ODO3397 | | | | lung | |
| asthmatic lung | 29325 | 7.15 | 14.30 | asthmatic | −3.682517483 |
| ODO4928 | | | | lung | |
| endo cells KC | control | 15.9 | 15.90 | endo cells | |
| endo VEGF KC | | 8.26 | 8.26 | endo VEGF | −1.924939467 |
| endo bFGF KC | | 2.01 | 2.01 | endo bFGF | −7.910447761 |
| heart Clontech | normal | 7.9 | 15.80 | heart | |
| heart (T-1) ischemic | 29417 | 67.47 | 134.94 | heart T-1 | 8.540506329 |
| heart (T-14) non- | 29422 | 106.83 | 213.66 | heart T-14 | 13.52278481 |
| obstructive DCM | | | | | |
| heart (T-3399) DCM | 29426 | 425.28 | 850.56 | heart T-3399 | 53.83291139 |
| adenoid GW99-269 | 26162 | 15.98 | 31.96 | adenoid | |
| tonsil GW98-280 | 22582 | 17.95 | 35.90 | tonsil | |
| T cells PC00314 | 28453 | 3.18 | 6.36 | T cells | |
| PBMNC KC | | 0 | 0.00 | PBMNC | |
| monocyte KC | | 0.81 | 1.62 | monocyte | |
| B cells PC00665 | 28455 | 2.74 | 5.48 | B cells | |
| dendritic cells 28441 | | 0 | 0.00 | dendritic | |
| | | | | cells | |
| neutrophils | 28440 | 0 | 0.00 | neutrophils | |
| eosinophils | 28446 | 0 | 0.00 | eosinophils | |
| BM unstim KC | | 0 | 0.00 | BM unstim | |
| BM stim KC | | 0 | 0.00 | BM stim | 0 |
| osteo dif KC | | 2.34 | 2.34 | osteo dif | |
| osteo undif KC | | 0 | 0.00 | osteo undif | −2.34 |
| chondrocytes | | 145.14 | 362.85 | chondrocytes | |
| OA Synovium IP12/01 | 29462 | 320.78 | 320.78 | OA | |
| | | | | Synovium | |
| OA Synovium NP10/01 | 29461 | 396.85 | 793.70 | OA | |
| | | | | Synovium | |
| OA Synovium NP57/00 | 28464 | 329.87 | 659.74 | OA | |
| | | | | Synovium | |
| RA Synovium NP03/01 | 28466 | 103.85 | 207.70 | RA | |
| | | | | Synovium | |
| RA Synovium NP71/00 | 28467 | 617.72 | 1235.44 | RA | |
| | | | | Synovium | |
| RA Synovium NP45/00 | 28475 | 63.13 | 126.26 | RA | |
| | | | | Synovium | |
| OA bone (biobank) | 29217 | 3.19 | 3.19 | OA bone | |
| | | | | (biobank) | |
| OA bone Sample 1 | J. Emory | 126.87 | 253.74 | OA bone | |
| OA bone Sample 2 | J. Emory | 44.76 | 89.52 | OA bone | |
| Cartilage (pool) | Normal | 502.66 | 1005.32 | Cartilage | |
| | | | | (pool) | |
| Cartilage (pool) | OA | 206.76 | 413.52 | Cartilage | −2.431127878 |
| | | | | (pool) | |
| PBL unifected | 28441 | 0 | 0.00 | PBL | |
| | | | | unifected | |
| PBL HIV IIIB | 28442 | 0 | 0.00 | PBL HIV | 0 |
| | | | | IIIB | |
| MRC5 uninfected | 29158 | 0 | 0.00 | MRC5 | |
| (100%) | | | | uninfected | |
| | | | | (100%) | |
| MRC5 HSV strain F | 29178 | 17.73 | 35.46 | MRC5 HSV | 35.46 |
| | | | | strain F | |
| W12 cells | 29179 | 0.62 | 1.24 | W12 cells | |
| Keratinocytes | 29180 | 22.63 | 45.26 | Keratinocytes | |

Gene Name sbg419582PROTOCADHERIN

| | Fold Change in Disease |
|---|---|
| | Population Relative to |
| Disease tissues | Normal |
| colon tumor | −5.52 |
| colon tumor | 1.36 |
| colon tumor | −1.73 |
| colon tumor | −8.84 |
| lung tumor | 1.69 |
| lung tumor | −3.36 |
| lung tumor | 85.66 |
| lung tumor | −7.56 |
| breast tumor | 1.48 |
| breast tumor | 16.70 |
| breast tumor | 7.17 |
| breast tumor | −6.29 |
| brain stage 5 ALZ | −8.27 |
| brain stage 5 ALZ | −1.25 |
| brain stage 5 ALZ | −2.05 |
| brain stage 5 ALZ | −1.33 |
| lung 24 | −35.82 |
| lung 28 | −52.66 |
| lung 23 | −27.57 |
| asthmatic lung | −19.65 |
| asthmatic lung | −8.10 |
| asthmatic lung | −1.00 |
| asthmatic lung | −3.68 |
| endo VEGF | −1.92 |
| endo bFGF | −7.91 |
| heart T-1 | 8.54 |
| heart T-14 | 13.52 |
| heart T-3399 | 53.83 |
| BM stim | 0.00 |
| osteo undif | −2.34 |
| Cartilage (pool) | −2.43 |
| PBL HIV IIIB | 0.00 |
| MRC5 HSV strain F | 35.46 |

Gene Name sbg453915TECTORINa

Very low expression overall. Expression in female reproductive tissues suggests a protein that may be secreted by these tissue types.

| | | | | | | copies of |
|---|---|---|---|---|---|---|
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg453915TECTORINa | (sample 1) | (sample 2) | Copies | (ng) | (ng) | RNA |
| Subcutaneous | 2.70 | 5.41 | 4.06 | 3.06 | 16.34 | 66.26 |
| Adipocytes Zenbio | | | | | | |
| Subcutaneous Adipose | 0.00 | 0.00 | 0.00 | 0.96 | 52.36 | 0.00 |
| Zenbio | | | | | | |
| Adrenal Gland Clontech | 3.75 | 5.67 | 4.71 | 0.61 | 81.97 | 386.07 |
| Whole Brain Clontech | 22.57 | 27.88 | 25.23 | 7.24 | 6.91 | 174.21 |
| Fetal Brain Clontech | 2.42 | 1.80 | 2.11 | 0.48 | 103.95 | 219.33 |
| Cerebellum Clontech | 0.00 | 1.93 | 0.97 | 2.17 | 23.04 | 22.24 |
| Cervix | 2.90 | 2.10 | 2.50 | 2.42 | 20.66 | 51.65 |
| Colon | 11.19 | 2.68 | 6.94 | 2.71 | 18.45 | 127.95 |
| Endometrium | 4.79 | 19.31 | 12.05 | 0.73 | 68.21 | 821.96 |
| Esophagus | 2.06 | 2.93 | 2.50 | 1.37 | 36.50 | 91.06 |
| Heart Clontech | 5.42 | 7.31 | 6.37 | 1.32 | 37.88 | 241.10 |
| Hypothalamus | 0.00 | 3.70 | 1.85 | 0.32 | 155.28 | 287.27 |
| Ileum | 3.72 | 18.75 | 11.24 | 2.58 | 19.38 | 217.73 |
| Jejunum | 28.49 | 49.80 | 39.15 | 6.60 | 7.58 | 296.55 |
| Kidney | 2.12 | 4.37 | 3.25 | 2.12 | 23.58 | 76.53 |
| Liver | 15.74 | 39.80 | 27.77 | 1.50 | 33.33 | 925.67 |
| Fetal Liver Clontech | 27.96 | 26.14 | 27.05 | 10.40 | 4.81 | 130.05 |
| Lung | 0.00 | 2.37 | 1.19 | 2.57 | 19.46 | 23.05 |
| Mammary Gland | 19.68 | 19.22 | 19.45 | 13.00 | 3.85 | 74.81 |
| Clontech | | | | | | |
| Myometrium | 3.40 | 1.71 | 2.56 | 2.34 | 21.37 | 54.59 |
| Omentum | 14.33 | 138.99 | 76.66 | 3.94 | 12.69 | 972.84 |
| Ovary | 46.55 | 37.80 | 42.18 | 4.34 | 11.52 | 485.89 |
| Pancreas | 4.26 | 2.19 | 3.23 | 0.81 | 61.80 | 199.32 |
| Head of Pancreas | 1.93 | 1.52 | 1.73 | 1.57 | 31.85 | 54.94 |
| Parotid Gland | 4.04 | 5.93 | 4.99 | 5.48 | 9.12 | 45.48 |
| Placenta Clontech | 3.69 | 15.48 | 9.59 | 5.26 | 9.51 | 91.11 |
| Prostate | 7.94 | 28.75 | 18.35 | 3.00 | 16.67 | 305.75 |
| Rectum | 11.09 | 3.41 | 7.25 | 1.23 | 40.65 | 294.72 |
| Salivary Gland | 0.00 | 1.45 | 0.73 | 7.31 | 6.84 | 4.96 |
| Clontech | | | | | | |
| Skeletal Muscle | 4.76 | 0.00 | 2.38 | 1.26 | 39.68 | 94.44 |
| Clontech | | | | | | |
| Skin | 0.00 | 1.39 | 0.70 | 1.21 | 41.32 | 28.72 |
| Small Intestine | 2.20 | 1.41 | 1.81 | 0.98 | 51.07 | 92.19 |
| Clontech | | | | | | |
| Spleen | 7.15 | 8.12 | 7.64 | 4.92 | 10.16 | 77.59 |
| Stomach | 1.98 | 0.00 | 0.99 | 2.73 | 18.32 | 18.13 |
| Testis Clontech | 6.83 | 2.61 | 4.72 | 0.57 | 87.87 | 414.76 |
| Thymus Clontech | 0.00 | 0.00 | 0.00 | 9.89 | 5.06 | 0.00 |
| Thyroid | 2.38 | 1.88 | 2.13 | 2.77 | 18.05 | 38.45 |
| Trachea Clontech | 1.71 | 9.25 | 5.48 | 9.71 | 5.15 | 28.22 |
| Urinary Bladder | 3.72 | 8.22 | 5.97 | 5.47 | 9.14 | 54.57 |
| Uterus | 74.31 | 73.54 | 73.93 | 5.34 | 9.36 | 692.18 |

| | | | copies of | | |
|---|---|---|---|---|---|
| | Reg | | mRNA | | |
| | number | Mean | detected/50 ng | | Fold Change in |
| Sample | (GSK | GOI | total | | Disease |
| sbg453915TECTORINa | identifier) | copies | RNA | Sample | Population |
| colon normal GW98-167 | 21941 | 131.15 | 262.30 | colon normal | |
| colon tumor GW98-166 | 21940 | 85.76 | 171.52 | colon tumor | −1.529267724 |
| colon normal GW98-178 | 22080 | 1.82 | 3.64 | colon normal | |
| colon tumor GW98-177 | 22060 | 10.14 | 20.28 | colon tumor | 5.571428571 |
| colon normal GW98-561 | 23514 | 14.25 | 28.50 | colon normal | |
| colon tumor GW98-560 | 23513 | 9.89 | 19.78 | colon tumor | −1.440849343 |
| colon normal GW98-894 | 24691 | 32.05 | 64.10 | colon normal | |
| colon tumor GW98-893 | 24690 | 53.06 | 106.12 | colon tumor | 1.655538222 |
| lung normal GW98-3 | 20742 | 6.9 | 13.80 | lung normal | |
| lung tumor GW98-2 | 20741 | 0.81 | 1.62 | lung tumor | −8.518518519 |
| lung normal GW97-179 | 20677 | 1.19 | 2.38 | lung normal | |
| lung tumor GW97-178 | 20676 | 0 | 0.00 | lung tumor | −2.38 |
| lung normal GW98-165 | 21922 | 0.91 | 1.82 | lung normal | |
| lung tumor GW98-164 | 21921 | 5.99 | 11.98 | lung tumor | 6.582417582 |
| lung normal GW98-282 | 22584 | 5.93 | 11.86 | lung normal | |
| lung tumor GW98-281 | 22583 | 1.54 | 3.08 | lung tumor | −3.850649351 |
| breast normal GW00-392 | 28750 | 6.88 | 6.88 | breast | |
| | | | | normal | |
| breast tumor GW00-391 | 28746 | 4.24 | 8.48 | breast tumor | 1.23255814 |
| breast normal GW00-413 | 28798 | 0 | 0.00 | breast | |
| | | | | normal | |
| breast tumor GW00-412 | 28797 | 13.96 | 27.92 | breast tumor | 27.92 |
| breast normal GW00- | 27592-95 | 14.42 | 14.42 | breast | |
| 235:238 | | | | normal | |
| breast tumor GW00- | 27588-91 | 0 | 0.00 | breast tumor | −14.42 |
| 231:234 | | | | | |
| breast normal GW98-621 | 23656 | 5.81 | 11.62 | breast | |
| | | | | normal | |
| breast tumor GW98-620 | 23655 | 0 | 0.00 | breast tumor | −11.62 |
| brain normal BB99-542 | 25507 | 20.59 | 41.18 | brain normal | |
| brain normal BB99-406 | 25509 | 15.98 | 31.96 | brain normal | |
| brain normal BB99-904 | 25546 | 2.38 | 4.76 | brain normal | |
| brain stage 5 ALZ BB99- | 25502 | 25.45 | 50.90 | brain stage 5 | 1.960205392 |
| 874 | | | | ALZ | |
| brain stage 5 ALZ BB99- | 25503 | 35.78 | 71.56 | brain stage 5 | 2.755840822 |
| 887 | | | | ALZ | |
| brain stage 5 ALZ BB99- | 25504 | 13.83 | 27.66 | brain stage 5 | 1.06521181 |
| 862 | | | | ALZ | |
| brain stage 5 ALZ BB99- | 25542 | 21.67 | 43.34 | brain stage 5 | 1.669062901 |
| 927 | | | | ALZ | |
| CT lung KC | normal | 6.52 | 13.04 | CT lung | |
| lung 26 KC | normal | 2.1 | 2.10 | lung 26 | |
| lung 27 KC | normal | 0.84 | 0.84 | lung 27 | |
| lung 24 KC | COPD | 1.25 | 1.25 | lung 24 | −3.432 |
| lung 28 KC | COPD | 0 | 0.00 | lung 28 | −4.29 |
| lung 23 KC | COPD | 1.16 | 1.16 | lung 23 | −3.698275862 |
| lung 25 KC | COPD | 1.18 | 1.18 | lung 25 | |
| asthmatic lung ODO3112 | 29321 | 4.9 | 4.90 | asthmatic | 1.142191142 |
| | | | | lung | |
| asthmatic lung ODO3433 | 29323 | 0.83 | 1.66 | asthmatic | −2.584337349 |
| | | | | lung | |
| asthmatic lung ODO3397 | 29322 | 2.46 | 4.92 | asthmatic | 1.146853147 |
| | | | | lung | |
| asthmatic lung ODO4928 | 29325 | 6 | 12.00 | asthmatic | 2.797202797 |
| | | | | lung | |
| endo cells KC | control | 2.52 | 2.52 | endo cells | |
| endo VEGF KC | | 1.28 | 1.28 | endo VEGF | −1.96875 |
| endo bFGF KC | | 0 | 0.00 | endo bFGF | −2.52 |
| heart Clontech | normal | 0 | 0.00 | heart | |
| heart (T-1) ischemic | 29417 | 3.58 | 7.16 | heart T-1 | 7.16 |
| heart (T-14) non- | 29422 | 0 | 0.00 | heart T-14 | 0 |
| obstructive DCM | | | | | |
| heart (T-3399)DCM | 29426 | 0 | 0.00 | heart T-3399 | 0 |
| adenoid GW99-269 | 26162 | 2.29 | 4.58 | adenoid | |
| tonsil GW98-280 | 22582 | 1.85 | 3.70 | tonsil | |
| T cells PC00314 | 28453 | 4.29 | 8.58 | T cells | |
| PBMNC KC | | 0 | 0.00 | PBMNC | |
| monocyte KC | | 3.39 | 6.78 | monocyte | |
| B cells PC00665 | 28455 | 6.04 | 12.08 | B cells | |
| dendritic cells 28441 | | 0.83 | 1.66 | dendritic | |
| | | | | cells | |
| neutrophils | 28440 | 34.69 | 34.69 | neutrophils | |
| eosinophils | 28446 | 2.86 | 5.72 | eosinophils | |
| BM unstim KC | | 0 | 0.00 | BM unstim | |
| BM stim KC | | 12.8 | 12.80 | BM stim | 12.8 |
| osteo dif KC | | 0 | 0.00 | osteo dif | |
| osteo undif KC | | 0 | 0.00 | osteo undif | 0 |
| chondrocytes | | 4.78 | 11.95 | chondrocytes | |
| OA Synovium IP12/01 | 29462 | 18.31 | 18.31 | OA | |
| | | | | Synovium | |
| OA Synovium NP10/01 | 29461 | 0 | 0.00 | OA | |
| | | | | Synovium | |
| OA Synovium NP57/00 | 28464 | 11.46 | 22.92 | OA | |
| | | | | Synovium | |
| RA Synovium NP03/01 | 28466 | 0.87 | 1.74 | RA | |
| | | | | Synovium | |
| RA Synovium NP71/00 | 28467 | 26.95 | 53.90 | RA | |
| | | | | Synovium | |
| RA Synovium NP45/00 | 28475 | 18.91 | 37.82 | RA | |
| | | | | Synovium | |
| OA bone (biobank) | 29217 | 0 | 0.00 | OA bone | |
| | | | | (biobank) | |
| OA bone Sample 1 | J. Emory | 8.66 | 17.32 | OA bone | |
| OA bone Sample 2 | J. Emory | 7.8 | 15.60 | OA bone | |
| Cartilage (pool) | Normal | 16.93 | 33.86 | Cartilage | |
| | | | | (pool) | |
| Cartilage (pool) | OA | 6.39 | 12.78 | Cartilage | −2.649452269 |
| | | | | (pool) | |
| PBL unifected | 28441 | 0 | 0.00 | PBL | |
| | | | | unifected | |
| PBL HIV IIIB | 28442 | 1.15 | 2.30 | PBL HIV | 2.3 |
| | | | | IIIB | |
| MRC5 uninfected | 29158 | 0 | 0.00 | MRC5 | |
| (100%) | | | | uninfected | |
| | | | | (100%) | |
| MRC5 HSV strain F | 29178 | 70.84 | 141.68 | MRC5 HSV | 141.68 |
| | | | | strain F | |
| W12 cells | 29179 | 5.59 | 11.18 | W12 cells | |
| Keratinocytes | 29180 | 0 | 0.00 | Keratinocytes | |

Gene Name sbg453915TECTORINa

| | Fold Change in Disease |
|---|---|
| | Population Relative to |
| Disease tissues | Normal |
| colon tumor | −1.53 |
| colon tumor | 5.57 |
| colon tumor | −1.44 |
| colon tumor | 1.66 |
| lung tumor | −8.52 |
| lung tumor | −2.38 |
| lung tumor | 6.58 |
| lung tumor | −3.85 |
| breast tumor | 1.23 |
| breast tumor | 27.92 |
| breast tumor | −14.42 |
| breast tumor | −11.62 |
| brain stage 5 ALZ | 1.96 |
| brain stage 5 ALZ | 2.76 |
| brain stage 5 ALZ | 1.07 |
| brain stage 5 ALZ | 1.67 |
| lung 24 | −3.43 |
| lung 28 | −4.29 |
| lung 23 | −3.70 |
| asthmatic lung | 1.14 |
| asthmatic lung | −2.58 |
| asthmatic lung | 1.15 |
| asthmatic lung | 2.80 |
| endo VEGF | −1.97 |
| endo bFGF | −2.52 |
| heart T-1 | 7.16 |
| heart T-14 | 0.00 |
| heart T-3399 | 0.00 |
| BM stim | 12.80 |
| osteo undif | 0.00 |
| Cartilage (pool) | −2.65 |
| PBL HIV IIIB | 2.30 |
| MRC5 HSV strain F | 141.68 |

Gene Name SBh385630.antiinflam

Some expression in adenoid, tonsils and T-cells suggesting a role in the immune system. Expression in GI tissues suggests a role in the digestive system and potential role in diseases of the GI system such as EBD. Overexpression in lung (1/4) and colon tumors (1/4) suggesting a role in lung and colon cancer. Increased expression in ischemic and dilated heart samples indicating a role in Cardiovascular diseases that are consistent with cardiac hypertropby. Expression in whole brain but not localized to hypothalamus, cerebellum or cortex.

| | | | | | | copies of |
|---|---|---|---|---|---|---|
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| SBh385630.antiinflam | (sample 1) | (sample 2) | Copies | (ng) | (ng) | RNA |
| Subcutaneous | 0.00 | 6.41 | 3.21 | 3.06 | 16.34 | 52.37 |
| Adipocytes Zenbio | | | | | | |
| Subcutaneous Adipose | 0.00 | 0.00 | 0.00 | 0.96 | 52.36 | 0.00 |
| Zenbio | | | | | | |
| Adrenal Gland Clontech | 8.40 | 0.00 | 4.20 | 0.61 | 81.97 | 344.26 |
| Whole Brain Clontech | 817.17 | 466.76 | 641.97 | 7.24 | 6.91 | 4433.46 |
| Fetal Brain Clontech | 3.80 | 0.00 | 1.90 | 0.48 | 103.95 | 197.51 |
| Cerebellum Clontech | 6.66 | 0.00 | 3.33 | 2.17 | 23.04 | 76.73 |
| Cervix | 11.99 | 12.30 | 12.15 | 2.42 | 20.66 | 250.93 |
| Colon | 55.51 | 211.32 | 133.42 | 2.71 | 18.45 | 2461.53 |
| Endometrium | 0.00 | 0.00 | 0.00 | 0.73 | 68.21 | 0.00 |
| Esophagus | 11.75 | 30.29 | 21.02 | 1.37 | 36.50 | 767.15 |
| Heart Clontech | 0.00 | 0.00 | 0.00 | 1.32 | 37.88 | 0.00 |
| Hypothalamus | 0.00 | 0.00 | 0.00 | 0.32 | 155.28 | 0.00 |
| Ileum | 40.37 | 42.85 | 41.61 | 2.58 | 19.38 | 806.40 |
| Jejunum | 200.19 | 263.82 | 232.01 | 6.60 | 7.58 | 1757.61 |
| Kidney | 18.38 | 34.53 | 26.46 | 2.12 | 23.58 | 623.94 |
| Liver | 11.00 | 17.20 | 14.10 | 1.50 | 33.33 | 470.00 |
| Fetal Liver Clontech | 150.74 | 123.93 | 137.34 | 10.40 | 4.81 | 660.26 |
| Lung | 82.73 | 77.24 | 79.99 | 2.57 | 19.46 | 1556.13 |
| Mammary Gland | 161.37 | 155.19 | 158.28 | 13.00 | 3.85 | 608.77 |
| Clontech | | | | | | |
| Myometrium | 5.79 | 9.38 | 7.59 | 2.34 | 21.37 | 162.07 |
| Omentum | 36.14 | 46.80 | 41.47 | 3.94 | 12.69 | 526.27 |
| Ovary | 59.25 | 44.29 | 51.77 | 4.34 | 11.52 | 596.43 |
| Pancreas | 6.29 | 6.70 | 6.50 | 0.81 | 61.80 | 401.42 |
| Head of Pancreas | 0.00 | 26.25 | 13.13 | 1.57 | 31.85 | 417.99 |
| Parotid Gland | 8.77 | 52.96 | 30.87 | 5.48 | 9.12 | 281.61 |
| Placenta Clontech | 4.11 | 0.00 | 2.06 | 5.26 | 9.51 | 19.53 |
| Prostate | 100.91 | 49.99 | 75.45 | 3.00 | 16.67 | 1257.50 |
| Rectum | 180.24 | 305.61 | 242.93 | 1.23 | 40.65 | 9875.00 |
| Salivary Gland Clontech | 49.36 | 70.01 | 59.69 | 7.31 | 6.84 | 408.24 |
| Skeletal Muscle | 0.00 | 0.00 | 0.00 | 1.26 | 39.68 | 0.00 |
| Clontech | | | | | | |
| Skin | 18.00 | 3.22 | 10.61 | 1.21 | 41.32 | 438.43 |
| Small Intestine Clontech | 3.90 | 2.55 | 3.23 | 0.98 | 51.07 | 164.71 |
| Spleen | 9.67 | 5.60 | 7.64 | 4.92 | 10.16 | 77.59 |
| Stomach | 32.34 | 83.60 | 57.97 | 2.73 | 18.32 | 1061.72 |
| Testis Clontech | 3.53 | 0.00 | 1.77 | 0.57 | 87.87 | 155.10 |
| Thymus Clontech | 73.66 | 60.02 | 66.84 | 9.89 | 5.06 | 337.92 |
| Thyroid | 15.87 | 12.31 | 14.09 | 2.77 | 18.05 | 254.33 |
| Trachea Clontech | 98.68 | 187.11 | 142.90 | 9.71 | 5.15 | 735.81 |
| Urinary Bladder | 118.92 | 101.91 | 110.42 | 5.47 | 9.14 | 1009.28 |
| Uterus | 9.03 | 24.21 | 16.62 | 5.34 | 9.36 | 155.62 |

| | | | copies of | | |
|---|---|---|---|---|---|
| | Reg | | mRNA | | |
| | number | Mean | detected/50 ng | | Fold Change in |
| Sample | (GSK | GOI | total | | Disease |
| SBh385630.antiinflam | identifier) | copies | RNA | Sample | Population |
| colon normal GW98-167 | 21941 | 6479.77 | 12959.54 | colon normal | |
| colon tumor GW98-166 | 21940 | 7824.02 | 15648.04 | colon tumor | 1.207453351 |
| colon normal GW98-178 | 22080 | 343.81 | 687.62 | colon normal | |
| colon tumor GW98-177 | 22060 | 3011.93 | 6023.86 | colon tumor | 8.760449085 |
| colon normal GW98-561 | 23514 | 5457.38 | 10914.76 | colon normal | |
| colon tumor GW98-560 | 23513 | 4017.14 | 8034.28 | colon tumor | −1.358523726 |
| colon normal GW98-894 | 24691 | 14903.68 | 29807.36 | colon normal | |
| colon tumor GW98-893 | 24690 | 4814.19 | 9628.38 | colon tumor | −3.095781429 |
| lung normal GW98-3 | 20742 | 3731.84 | 7463.68 | lung normal | |
| lung tumor GW98-2 | 20741 | 719.6 | 1439.20 | lung tumor | −5.185992218 |
| lung normal GW97-179 | 20677 | 1090.56 | 2181.12 | lung normal | |
| lung tumor GW97-178 | 20676 | 6187.22 | 12374.44 | lung tumor | 5.673433832 |
| lung normal GW98-165 | 21922 | 8416.82 | 16833.64 | lung normal | |
| lung tumor GW98-164 | 21921 | 4405.14 | 8810.28 | lung tumor | −1.910681613 |
| lung normal GW98-282 | 22584 | 2033.26 | 4066.52 | lung normal | |
| lung tumor GW98-281 | 22583 | 1785.69 | 3571.38 | lung tumor | −1.138641086 |
| breast normal GW00-392 | 28750 | 1583.49 | 1583.49 | breast | |
| | | | | normal | |
| breast tumor GW00-391 | 28746 | 1334.89 | 2669.78 | breast tumor | 1.686010016 |
| breast normal GW00-413 | 28798 | 1225.92 | 1225.92 | breast | |
| | | | | normal | |
| breast tumor GW00-412 | 28797 | 1213.71 | 2427.42 | breast tumor | 1.980080266 |
| breast normal GW00- | 27592-95 | 862.26 | 862.26 | breast | |
| 235:238 | | | | normal | |
| breast tumor GW00- | 27588-91 | 1766.08 | 1766.08 | breast tumor | 2.048198919 |
| 231:234 | | | | | |
| breast normal GW98-621 | 23656 | 1420.57 | 2841.14 | breast | |
| | | | | normal | |
| breast tumor GW98-620 | 23655 | 760.05 | 1520.10 | breast tumor | −1.869048089 |
| brain normal BB99-542 | 25507 | 679.48 | 1358.96 | brain normal | |
| brain normal BB99-406 | 25509 | 423.69 | 847.38 | brain normal | |
| brain normal BB99-904 | 25546 | 401.34 | 802.68 | brain normal | |
| brain stage 5 ALZ BB99- | 25502 | 264.51 | 529.02 | brain stage 5 | −1.895971167 |
| 874 | | | | ALZ | |
| brain stage 5 ALZ BB99- | 25503 | 648.88 | 1297.76 | brain stage 5 | 1.293869765 |
| 887 | | | | ALZ | |
| brain stage 5 ALZ BB99- | 25504 | 234.97 | 469.94 | brain stage 5 | −2.134329205 |
| 862 | | | | ALZ | |
| brain stage 5 ALZ BB99- | 25542 | 404.55 | 809.10 | brain stage 5 | −1.239657232 |
| 927 | | | | ALZ | |
| CT lung KC | normal | 6620.85 | 13241.70 | CT lung | |
| lung 26 KC | normal | 320.43 | 320.43 | lung 26 | |
| lung 27 KC | normal | 164.59 | 164.59 | lung 27 | |
| lung 24 KC | COPD | 141.57 | 141.57 | lung 24 | −25.25392032 |
| lung 28 KC | COPD | 323.8 | 323.80 | lung 28 | −11.04137585 |
| lung 23 KC | COPD | 363.35 | 363.35 | lung 23 | −9.839541764 |
| lung 25 KC | COPD | 574.07 | 574.07 | lung 25 | |
| asthmatic lung | 29321 | 6073.99 | 6073.99 | asthmatic | 1.698924325 |
| ODO3112 | | | | lung | |
| asthmatic lung | 29323 | 4568.41 | 9136.82 | asthmatic | 2.555612662 |
| ODO3433 | | | | lung | |
| asthmatic lung | 29322 | 17389.11 | 34778.22 | asthmatic | 9.727636026 |
| ODO3397 | | | | lung | |
| asthmatic lung | 29325 | 4719.27 | 9438.54 | asthmatic | 2.640005203 |
| ODO4928 | | | | lung | |
| endo cells KC | control | 0 | 0.00 | endo cells | |
| endo VEGF KC | | 0 | 0.00 | endo VEGF | 0 |
| endo bFGF KC | | 0 | 0.00 | endo bFGF | 0 |
| heart Clontech | normal | 10.63 | 21.26 | heart | |
| heart (T-1) ischemic | 29417 | 599.01 | 1198.02 | heart T-1 | 56.3508937 |
| heart (T-14) non- | 29422 | 666.41 | 1332.82 | heart T-14 | 62.69143932 |
| obstructive DCM | | | | | |
| heart (T-3399) DCM | 29426 | 142.85 | 285.70 | heart T-3399 | 13.43838194 |
| adenoid GW99-269 | 26162 | 1138 | 2276.00 | adenoid | |
| tonsil GW98-280 | 22582 | 561.57 | 1123.14 | tonsil | |
| T cells PC00314 | 28453 | 736.27 | 1472.54 | T cells | |
| PBMNC KC | | 0 | 0.00 | PBMNC | |
| monocyte KC | | 30.38 | 60.76 | monocyte | |
| B cells PG00665 | 28455 | 204.15 | 408.30 | B cells | |
| dendritic cells 28441 | | 57.66 | 115.32 | dendritic | |
| | | | | cells | |
| neutrophils | 28440 | 13.3 | 13.30 | neutrophils | |
| eosinophils | 28446 | 5.71 | 11.42 | eosinophils | |
| BM unstim KC | | 0 | 0.00 | BM unstim | |
| BM stim KC | | 50.38 | 50.38 | BM stim | 50.38 |
| osteo dif KC | | 8.62 | 8.62 | osteo dif | |
| osteo undif KC | | 0 | 0.00 | osteo undif | −8.62 |
| chondrocytes | | 14.98 | 37.45 | chondrocytes | |
| OA Synovium IP12/01 | 29462 | 134.63 | 134.63 | OA | |
| | | | | Synovium | |
| OA Synovium NP10/01 | 29461 | 73.89 | 147.78 | OA | |
| | | | | Synovium | |
| OA Synovium NP57/00 | 28464 | 106.98 | 213.96 | OA | |
| | | | | Synovium | |
| RA Synovium NP03/01 | 28466 | 26.59 | 53.18 | RA | |
| | | | | Synovium | |
| RA Synovium NP71/00 | 28467 | 60.88 | 121.76 | RA | |
| | | | | Synovium | |
| RA Synovium NP45/00 | 28475 | 60.81 | 121.62 | RA | |
| | | | | Synovium | |
| OA bone (biobank) | 29217 | 98.18 | 98.18 | OA bone | |
| | | | | (biobank) | |
| OA bone Sample 1 | J. Emory | 78.3 | 156.60 | OA bone | |
| OA bone Sample 2 | J. Emory | 107.7 | 215.40 | OA bone | |
| Cartilage (pool) | Normal | 72.21 | 144.42 | Cartilage | |
| | | | | (pool) | |
| Cartilage (pool) | OA | 48.61 | 97.22 | Cartilage | −1.485496811 |
| | | | | (pool) | |
| PBL unifected | 28441 | 30.22 | 60.44 | PBL | |
| | | | | unifected | |
| PBL HIV IIIB | 28442 | 21.89 | 43.78 | PBL HIV | −1.380539059 |
| | | | | IIIB | |
| MRC5 uninfected | 29158 | 10.74 | 21.48 | MRC5 | |
| (100%) | | | | uninfected | |
| | | | | (100%) | |
| MRC5 HSV strain F | 29178 | 171.23 | 342.46 | MRC5 HSV | 15.94320298 |
| | | | | strain F | |
| W12 cells | 29179 | 1143.85 | 2287.70 | W12 cells | |
| Keratinocytes | 29180 | 388.06 | 776.12 | Keratinocytes | |

Gene Name SBh385630.antiinflam

| | Fold Change in Disease |
|---|---|
| | Population Relative to |
| Disease tissues | Normal |
| colon tumor | 1.21 |
| colon tumor | 8.76 |
| colon tumor | −1.36 |
| colon tumor | −3.10 |
| lung tumor | −5.19 |
| lung tumor | 5.67 |
| lung tumor | −1.91 |
| lung tumor | −1.14 |
| breast tumor | 1.69 |
| breast tumor | 1.98 |
| breast tumor | 2.05 |
| breast tumor | −1.87 |
| brain stage 5 ALZ | −1.90 |
| brain stage 5 ALZ | 1.29 |
| brain stage 5 ALZ | −2.13 |
| brain stage 5 ALZ | −1.24 |
| lung 24 | −25.25 |
| lung 28 | −11.04 |
| lung 23 | −9.84 |
| asthmatic lung | 1.70 |
| asthmatic lung | 2.56 |
| asthmatic lung | 9.73 |
| asthmatic lung | 2.64 |
| endo VEGF | 0.00 |
| endo bFGF | 0.00 |
| heart T-1 | 56.35 |
| heart T-14 | 62.69 |
| heart T-3399 | 13.44 |
| BM stim | 50.38 |
| osteo undif | −8.62 |
| Cartilage (pool) | −1.49 |
| PBL HIV IIIB | −1.38 |
| MRC5 HSV strain F | 15.94 |

Gene Name sbg471005nAChR

Expressed in immune cells with corroborating expression in OA and RA synovium suggesting a role in this disease.

High expression in whole brain but not present in cortex, cerebellum, or hypothalamus suggesting localized brain expression.

| | | | | | | copies |
|---|---|---|---|---|---|---|
| | | | | | | of |
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg471005nAChR | (sample 1) | (sample 2) | Copies | (ng) | (ng) | RNA |
| Subcutaneous | 32.42 | 2.90 | 17.66 | 3.06 | 16.34 | 288.56 |
| Adipocytes Zenbio | | | | | | |
| Subcutaneous Adipose | 0.00 | 0.00 | 0.00 | 0.96 | 52.36 | 0.00 |
| Zenbio | | | | | | |
| Adrenal Gland Clontech | 0.00 | 0.00 | 0.00 | 0.61 | 81.97 | 0.00 |
| Whole Brain Clontech | 1606.00 | 1058.07 | 1332.04 | 7.24 | 6.91 | 9199.14 |
| Fetal Brain Clontech | 0.00 | 6.34 | 3.17 | 0.48 | 103.95 | 329.52 |
| Cerebellum Clontech | 10.65 | 0.00 | 5.33 | 2.17 | 23.04 | 122.70 |
| Cervix | 0.00 | 0.00 | 0.00 | 2.42 | 20.66 | 0.00 |
| Colon | 0.00 | 0.00 | 0.00 | 2.71 | 18.45 | 0.00 |
| Endometrium | 0.00 | 0.00 | 0.00 | 0.73 | 68.21 | 0.00 |
| Esophagus | 0.00 | 2.52 | 1.26 | 1.37 | 36.50 | 45.99 |
| Heart Clontech | 4.05 | 0.00 | 2.03 | 1.32 | 37.88 | 76.70 |
| Hypothalamus | 2.24 | 0.00 | 1.12 | 0.32 | 155.28 | 173.91 |
| Ileum | 0.00 | 0.00 | 0.00 | 2.58 | 19.38 | 0.00 |
| Jejunum | 20.32 | 41.44 | 30.88 | 6.60 | 7.58 | 233.94 |
| Kidney | 14.56 | 0.00 | 7.28 | 2.12 | 23.58 | 171.70 |
| Liver | 3.55 | 10.72 | 7.14 | 1.50 | 33.33 | 237.83 |
| Fetal Liver Clontech | 127.95 | 116.81 | 122.38 | 10.40 | 4.81 | 588.37 |
| Lung | 12.79 | 0.00 | 6.40 | 2.57 | 19.46 | 124.42 |
| Mammary Gland | 30.53 | 24.12 | 27.33 | 13.00 | 3.85 | 105.10 |
| Clontech | | | | | | |
| Myometrium | 0.00 | 7.10 | 3.55 | 2.34 | 21.37 | 75.85 |
| Omentum | 8.15 | 0.00 | 4.08 | 3.94 | 12.69 | 51.71 |
| Ovary | 18.27 | 7.02 | 12.65 | 4.34 | 11.52 | 145.68 |
| Pancreas | 0.00 | 0.00 | 0.00 | 0.81 | 61.80 | 0.00 |
| Head of Pancreas | 0.00 | 0.00 | 0.00 | 1.57 | 31.85 | 0.00 |
| Parotid Gland | 0.00 | 0.00 | 0.00 | 5.48 | 9.12 | 0.00 |
| Placenta Clontech | 9.17 | 0.00 | 4.59 | 5.26 | 9.51 | 43.58 |
| Prostate | 0.00 | 1.35 | 0.68 | 3.00 | 16.67 | 11.25 |
| Rectum | 0.00 | 0.00 | 0.00 | 1.23 | 40.65 | 0.00 |
| Salivary Gland | 0.00 | 11.84 | 5.92 | 7.31 | 6.84 | 40.49 |
| Clontech | | | | | | |
| Skeletal Muscle | 6.09 | 7.36 | 6.73 | 1.26 | 39.68 | 266.87 |
| Clontech | | | | | | |
| Skin | 0.00 | 0.00 | 0.00 | 1.21 | 41.32 | 0.00 |
| Small Intestine | 0.00 | 0.00 | 0.00 | 0.98 | 51.07 | 0.00 |
| Clontech | | | | | | |
| Spleen | 5.20 | 7.36 | 6.28 | 4.92 | 10.16 | 63.82 |
| Stomach | 12.85 | 6.38 | 9.62 | 2.73 | 18.32 | 176.10 |
| Testis Clontech | 0.00 | 2.25 | 1.13 | 0.57 | 87.87 | 98.86 |
| Thymus Clontech | 177.85 | 168.23 | 173.04 | 9.89 | 5.06 | 874.82 |
| Thyroid | 6.44 | 0.00 | 3.22 | 2.77 | 18.05 | 58.12 |
| Trachea Clontech | 5.07 | 0.00 | 2.54 | 9.71 | 5.15 | 13.05 |
| Urinary Bladder | 0.00 | 0.00 | 0.00 | 5.47 | 9.14 | 0.00 |
| Uterus | 29.20 | 10.39 | 19.80 | 5.34 | 9.36 | 185.35 |

| | | | copies of | | |
|---|---|---|---|---|---|
| | Reg | | mRNA | | |
| | number | Mean | detected/50 ng | | Fold Change |
| Sample | (GSK | GOI | total | | in Disease |
| sbg471005nAChR | identifier) | copies | RNA | Sample | Population |
| colon normal GW98-167 | 21941 | 1530.09 | 3060.18 | colon normal | |
| colon tumor GW98-166 | 21940 | 617.15 | 1234.30 | colon tumor | −2.479283805 |
| colon normal GW98-178 | 22080 | 406.03 | 812.06 | colon normal | |
| colon tumor GW98-177 | 22060 | 1231.53 | 2463.06 | colon tumor | 3.033101002 |
| colon normal GW98-561 | 23514 | 844.37 | 1688.74 | colon normal | |
| colon tumor GW98-560 | 23513 | 633.99 | 1267.98 | colon tumor | −1.331834887 |
| colon normal GW98-894 | 24691 | 1130.51 | 2261.02 | colon normal | |
| colon tumor GW98-893 | 24690 | 721.29 | 1442.58 | colon tumor | −1.567344619 |
| lung normal GW98-3 | 20742 | 2433.65 | 4867.30 | lung normal | |
| lung tumor GW98-2 | 20741 | 334.04 | 668.08 | lung tumor | −7.28550473 |
| lung normal GW97-179 | 20677 | 823.51 | 1647.02 | lung normal | |
| lung tumor GW97-178 | 20676 | 1492 | 2984.00 | lung tumor | 1.811756991 |
| lung normal GW98-165 | 21922 | 829.65 | 1659.30 | lung normal | |
| lung tumor GW98-164 | 21921 | 595.31 | 1190.62 | lung tumor | −1.393643648 |
| lung normal GW98-282 | 22584 | 357.69 | 715.38 | lung normal | |
| lung tumor GW98-281 | 22583 | 256.76 | 513.52 | lung tumor | −1.393090824 |
| breast normal GW00-392 | 28750 | 357.44 | 357.44 | breast | |
| | | | | normal | |
| breast tumor GW00-391 | 28746 | 280.98 | 561.96 | breast tumor | 1.572179946 |
| breast normal GW00-413 | 28798 | 286.18 | 286.18 | breast | |
| | | | | normal | |
| breast tumor GW00-412 | 28797 | 195.5 | 391.00 | breast tumor | 1.366272975 |
| breast normal GW00- | 27592-95 | 161.68 | 161.68 | breast | |
| 235:238 | | | | normal | |
| breast tumor GW00- | 27588-91 | 217.83 | 217.83 | breast tumor | 1.347290945 |
| 231:234 | | | | | |
| breast normal GW98-621 | 23656 | 531.53 | 1063.06 | breast | |
| | | | | normal | |
| breast tumor GW98-620 | 23655 | 556.17 | 1112.34 | breast tumor | 1.046356744 |
| brain normal BB99-542 | 25507 | 143.72 | 287.44 | brain normal | |
| brain normal BB99-406 | 25509 | 569.17 | 1138.34 | brain normal | |
| brain normal BB99-904 | 25546 | 106.85 | 213.70 | brain normal | |
| brain stage 5 ALZ BB99- | 25502 | 286.37 | 572.74 | brain stage 5 | 1.048027423 |
| 874 | | | | ALZ | |
| brain stage 5 ALZ BB99- | 25503 | 746.74 | 1493.48 | brain stage 5 | 2.732842121 |
| 887 | | | | ALZ | |
| brain stage 5 ALZ BB99- | 25504 | 382.97 | 765.94 | brain stage 5 | 1.401554151 |
| 862 | | | | ALZ | |
| brain stage 5 ALZ BB99- | 25542 | 367.49 | 734.98 | brain stage 5 | 1.344902042 |
| 927 | | | | ALZ | |
| CT lung KC | normal | 175.41 | 350.82 | CT lung | |
| lung 26 KC | normal | 20.66 | 20.66 | lung 26 | |
| lung 27 KC | normal | 13.06 | 13.06 | lung 27 | |
| lung 24 KC | COPD | 15.89 | 15.89 | lung 24 | −6.182662052 |
| lung 28 KC | COPD | 7.34 | 7.34 | lung 28 | −13.38453678 |
| lung 23 KC | COPD | 22.3 | 22.30 | lung 23 | −4.405493274 |
| lung 25 KC | COPD | 8.43 | 8.43 | lung 25 | |
| asthmatic lung | 29321 | 264.47 | 264.47 | asthmatic | 2.692012113 |
| ODO3112 | | | | lung | |
| asthmatic lung | 29323 | 442.3 | 884.60 | asthmatic | 9.004249688 |
| ODO3433 | | | | lung | |
| asthmatic lung | 29322 | 670.04 | 1340.08 | asthmatic | 13.64053236 |
| ODO3397 | | | | lung | |
| asthmatic lung | 29325 | 414.13 | 828.26 | asthmatic | 8.430770797 |
| ODO4928 | | | | lung | |
| endo cells KC | control | 66.94 | 66.94 | endo cells | |
| endo VEGF KC | | 18.49 | 18.49 | endo VEGF | −3.620335316 |
| endo bFGF KC | | 15.93 | 15.93 | endo bFGF | −4.202134338 |
| heart Clontech | normal | 180.76 | 361.52 | heart | |
| heart (T-1) ischemic | 29417 | 161.9 | 323.80 | heart T-1 | −1.116491662 |
| heart (T-14) non- | 29422 | 141.03 | 282.06 | heart T-14 | −1.281713111 |
| obstructive DCM | | | | | |
| heart (T-3399) DCM | 29426 | 321.32 | 642.64 | heart T-3399 | 1.777605665 |
| adenoid GW99-269 | 26162 | 193.61 | 387.22 | adenoid | |
| tonsil GW98-280 | 22582 | 625.4 | 1250.80 | tonsil | |
| T cells PC00314 | 28453 | 140.44 | 280.88 | T cells | |
| PBMNC KC | | 0 | 0.00 | PBMNC | |
| monocyte KC | | 0 | 0.00 | monocyte | |
| B cells PC00665 | 28455 | 476.72 | 953.44 | B cells | |
| dendritic cells 28441 | | 205.79 | 411.58 | dendritic | |
| | | | | cells | |
| neutrophils | 28440 | 1366.99 | 1366.99 | neutrophils | |
| eosinophils | 28446 | 316.57 | 633.14 | eosinophils | |
| BM unstim KC | | 29.41 | 29.41 | BM unstim | |
| BM stim KC | | 46.03 | 46.03 | BM stim | 1.565113907 |
| osteo dif KC | | 17.47 | 17.47 | osteo dif | |
| osteo undif KC | | 1.87 | 1.87 | osteo undif | −9.342245989 |
| chondrocytes | | 735.88 | 1839.70 | chondrocytes | |
| OA Synovium IP12/01 | 29462 | 686.8 | 686.80 | OA | |
| | | | | Synovium | |
| OA Synovium NP10/01 | 29461 | 4887.16 | 9774.32 | OA | |
| | | | | Synovium | |
| OA Synovium NP57/00 | 28464 | 721.49 | 1442.98 | OA | |
| | | | | Synovium | |
| RA Synovium NP03/01 | 28466 | 383.33 | 766.66 | RA | |
| | | | | Synovium | |
| RA Synovium NP71/00 | 28467 | 780.94 | 1561.88 | RA | |
| | | | | Synovium | |
| RA Synovium NP45/00 | 28475 | 543.62 | 1087.24 | RA | |
| | | | | Synovium | |
| OA bone (biobank) | 29217 | 780.12 | 780.12 | OA bone | |
| | | | | (biobank) | |
| OA bone Sample 1 | J. Emory | 361.65 | 723.30 | OA bone | |
| OA bone Sample 2 | J. Emory | 197.57 | 395.14 | OA bone | |
| Cartilage (pool) | Normal | 220.7 | 441.40 | Cartilage | |
| | | | | (pool) | |
| Cartilage (pool) | OA | 75.52 | 151.04 | Cartilage | −2.922404661 |
| | | | | (pool) | |
| PBL unifected | 28441 | 1745.81 | 3491.62 | PBL | |
| | | | | unifected | |
| PBL HIV IIIB | 28442 | 832.4 | 1664.80 | PBL HIV | −2.097321 |
| | | | | IIIB | |
| MRC5 uninfected | 29158 | 147.92 | 295.84 | MRC5 | |
| (100%) | | | | uninfected | |
| | | | | (100%) | |
| MRC5 HSV strain F | 29178 | 146 | 292.00 | MRC5 HSV | −1.013150685 |
| | | | | strain F | |
| W12 cells | 29179 | 304.27 | 608.54 | W12 cells | |
| Keratinocytes | 29180 | 139.44 | 278.88 | Keratinocytes | |

Gene Name sbg471005nAChR

| | Fold Change in Disease |
|---|---|
| | Population Relative to |
| Disease tissues | Normal |
| colon tumor | −2.48 |
| colon tumor | 3.03 |
| colon tumor | −1.33 |
| colon tumor | −1.57 |
| lung tumor | −7.29 |
| lung tumor | 1.81 |
| lung tumor | −1.39 |
| lung tumor | −1.39 |
| breast tumor | 1.57 |
| breast tumor | 1.37 |
| breast tumor | 1.35 |
| breast tumor | 1.05 |
| brain stage 5 ALZ | 1.05 |
| brain stage 5 ALZ | 2.73 |
| brain stage 5 ALZ | 1.40 |
| brain stage 5 ALZ | 1.34 |
| lung 24 | −6.18 |
| lung 28 | −13.38 |
| lung 23 | −4.41 |
| asthmatic lung | 2.69 |
| asthmatic lung | 9.00 |
| asthmatic lung | 13.64 |
| asthmatic lung | 8.43 |
| endo VEGF | −3.62 |
| endo bFGF | −4.20 |
| heart T-1 | −1.12 |
| heart T-14 | −1.28 |
| heart T-3399 | 1.78 |
| BM stim | 1.57 |
| osteo undif | −9.34 |
| Cartilage (pool) | −2.92 |
| PBL HIV IIIB | −2.10 |
| MRC5 HSV strain F | −1.01 |

Gene Name sbg442445PROa

Strong expression in B-cells with expression in other immune cell types indicate function in immune system. Corroborating expression in RA and OA samples indicate role in disease. 2× increase in cells infected with HIV suggests possible marker in HIV infection. Expression in whole brain but not cortex or cerebellum suggests localized expression in brain.

| | | | | | | copies |
|---|---|---|---|---|---|---|
| | | | | | | of |
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg442445PROa | (sample 1) | (sample 2) | Copies | (ng) | (ng) | RNA |
| Subcutaneous | 1.13 | 3.82 | 2.48 | 3.06 | 16.34 | 40.44 |
| Adipocytes Zenbio | | | | | | |
| Subcutaneous Adipose | 0.63 | 0 | 0.32 | 0.96 | 52.36 | 16.49 |
| Zenbio | | | | | | |
| Adrenal Gland Clontech | 0.64 | 0.74 | 0.69 | 0.61 | 81.97 | 56.56 |
| Whole Brain Clontech | 368.87 | 396.51 | 382.69 | 7.24 | 6.91 | 2642.89 |
| Fetal Brain Clontech | 1.57 | 2.5 | 2.04 | 0.48 | 103.95 | 211.54 |
| Cerebellum Clontech | 1.63 | 0 | 0.82 | 2.17 | 23.04 | 18.78 |
| Cervix | 4.57 | 5.6 | 5.09 | 2.42 | 20.66 | 105.06 |
| Colon | 18.13 | 7.38 | 12.76 | 2.71 | 18.45 | 235.33 |
| Endometrium | 4.23 | 0 | 2.12 | 0.73 | 68.21 | 144.27 |
| Esophagus | 6.85 | 12.66 | 9.76 | 1.37 | 36.50 | 356.02 |
| Heart Clontech | 12.83 | 1.44 | 7.14 | 1.32 | 37.88 | 270.27 |
| Hypothalamus | 0.58 | 7.26 | 3.92 | 0.32 | 155.28 | 608.70 |
| Ileum | 22.89 | 6.34 | 14.62 | 2.58 | 19.38 | 283.24 |
| Jejunum | 6.67 | 36.71 | 21.69 | 6.60 | 7.58 | 164.32 |
| Kidney | 2.82 | 6.28 | 4.55 | 2.12 | 23.58 | 107.31 |
| Liver | 11.21 | 1.24 | 6.23 | 1.50 | 33.33 | 207.50 |
| Fetal Liver Clontech | 118 | 135.81 | 126.91 | 10.40 | 4.81 | 610.12 |
| Lung | 13.95 | 37.87 | 25.91 | 2.57 | 19.46 | 504.09 |
| Mammary Gland | 15.77 | 11.19 | 13.48 | 13.00 | 3.85 | 51.85 |
| Clontech | | | | | | |
| Myometriun | 16.26 | 49.21 | 32.74 | 2.34 | 21.37 | 699.47 |
| Omeatum | 16.64 | 25.59 | 21.12 | 3.94 | 12.69 | 267.96 |
| Ovary | 4.98 | 7.48 | 6.23 | 4.34 | 11.52 | 71.77 |
| Pancreas | 1.23 | 0 | 0.62 | 0.81 | 61.80 | 38.01 |
| Head of Pancreas | 3.57 | 0 | 1.79 | 1.57 | 31.85 | 56.85 |
| Parotid Gland | 0.59 | 0 | 0.30 | 5.48 | 9.12 | 2.69 |
| Placenta Clontech | 2.67 | 2.75 | 2.71 | 5.26 | 9.51 | 25.76 |
| Prostate | 9.23 | 7.92 | 8.58 | 3.00 | 16.67 | 142.92 |
| Rectum | 2.62 | 4.28 | 3.45 | 1.23 | 40.65 | 140.24 |
| Salivary Gland | 1.02 | 14.59 | 7.81 | 7.31 | 6.84 | 53.39 |
| Clontech | | | | | | |
| Skeletal Muscle | 0 | 0.98 | 0.49 | 1.26 | 39.68 | 19.44 |
| Clontech | | | | | | |
| Skin | 2.72 | 0 | 1.36 | 1.21 | 41.32 | 56.20 |
| Small Intestine | 0.99 | 1 | 1.00 | 0.98 | 51.07 | 50.82 |
| Clontech | | | | | | |
| Spleen | 31.29 | 42.16 | 36.73 | 4.92 | 10.16 | 373.22 |
| Stomach | 15.74 | | 7.87 | 2.73 | 18.32 | 144.14 |
| Testis Clontech | 4.63 | 2.77 | 3.70 | 0.57 | 87.87 | 325.13 |
| Thymus Clontech | 503.91 | 615.6 | 559.76 | 9.89 | 5.06 | 2829.90 |
| Thyroid | 0.75 | 10.38 | 5.57 | 2.77 | 18.05 | 100.45 |
| Trachea Clontech | 65.95 | 52.98 | 59.47 | 9.71 | 5.15 | 306.20 |
| Urinary Bladder | 9.1 | 3.76 | 6.43 | 5.47 | 9.14 | 58.78 |
| Uterus | 13.88 | 4.35 | 9.12 | 5.34 | 9.36 | 85.35 |

| | | | copies of | | |
|---|---|---|---|---|---|
| | Reg | | mRNA | | |
| | number | Mean | detected/50 ng | | Fold Change |
| Sample | (GSK | GOI | total | | in Disease |
| sbg442445PROa | identifier) | copies | RNA | Sample | Population |
| colon normal GW98-167 | 21941 | 392.89 | 785.78 | colon normal | |
| colon tumor GW98-166 | 21940 | 466.75 | 933.50 | colon tumor | 1.18799155 |
| colon normal GW98-178 | 22080 | 113.54 | 227.08 | colon normal | |
| colon tumor GW98-177 | 22060 | 43.88 | 87.76 | colon tumor | −2.587511395 |
| colon normal GW98-561 | 23514 | 335.16 | 670.32 | colon normal | |
| colon tumor GW98-560 | 23513 | 173.85 | 347.70 | colon tumor | −1.927868852 |
| colon normal GW98-894 | 24691 | 288.76 | 577.52 | colon normal | |
| colon tumor GW98-893 | 24690 | 164.44 | 328.88 | colon tumor | −1.756020433 |
| lung normal GW98-3 | 20742 | 2119.16 | 4238.32 | lung normal | |
| lung tumor GW98-2 | 20741 | 33.63 | 67.26 | lung tumor | −63.01397562 |
| lung normal GW97-179 | 20677 | 1213.42 | 2426.84 | lung normal | |
| lung tumor GW97-178 | 20676 | 2011.79 | 4023.58 | lung tumor | 1.657950256 |
| lung normal GW98-165 | 21922 | 2088.93 | 4177.86 | lung normal | |
| lung tumor GW98-164 | 21921 | 862.54 | 1725.08 | lung tumor | −2.421835509 |
| lung normal GW98-282 | 22584 | 499.54 | 999.08 | lung normal | |
| lung tumor GW98-281 | 22583 | 946.36 | 1892.72 | lung tumor | 1.894462906 |
| breast normal GW00-392 | 28750 | 208.96 | 208.96 | breast normal | |
| breast tumor GW00-391 | 28746 | 259.34 | 518.68 | breast tumor | 2.48219755 |
| breast normal GW00-413 | 28798 | 65.02 | 65.02 | breast normal | |
| breast tumor GW00-412 | 28797 | 493.02 | 986.04 | breast tumor | 15.16517994 |
| breast normal GW00- | 27592-95 | 24.18 | 24.18 | breast normal | |
| 235:238 | | | | | |
| breast tumor GW00- | 27588-91 | 126.63 | 126.63 | breast tumor | 5.236972705 |
| 231:234 | | | | | |
| breast normal GW98-621 | 23656 | 536.09 | 1072.18 | breast normal | |
| breast tumor GW98-620 | 23655 | 203.7 | 407.40 | breast tumor | −2.631762396 |
| brain normal BB99-542 | 25507 | 88.47 | 176.94 | brain normal | |
| brain normal BB99-406 | 25509 | 147.87 | 295.74 | brain normal | |
| brain normal BB99-904 | 25546 | 35.13 | 70.26 | brain normal | |
| brain stage 5 ALZ BB99- | 25502 | 75.02 | 150.04 | brain stage 5 | −1.206211677 |
| 874 | | | | ALZ | |
| brain stage 5 ALZ BB99- | 25503 | 189 | 378.00 | brain stage 5 | 2.088628578 |
| 887 | | | | ALZ | |
| brain stage 5 ALZ BB99- | 25504 | 131.38 | 262.76 | brain stage 5 | 1.451873135 |
| 862 | | | | ALZ | |
| brain stage 5 ALZ BB99- | 25542 | 36.77 | 73.54 | brain stage 5 | −2.46097362 |
| 927 | | | | ALZ | |
| CT lung KC | normal | 1441.16 | 2882.32 | CT lung | |
| lung 26 KC | normal | 69.7 | 69.70 | lung 26 | |
| lung 27 KC | normal | 59.95 | 59.95 | lung 27 | |
| lung 24 KC | COPD | 5.33 | 5.33 | lung 24 | −142.0727017 |
| lung 28 KC | COPD | 30.24 | 30.24 | lung 28 | −25.04125331 |
| lung 23 KC | COPD | 52.96 | 52.96 | lung 23 | −14.29847998 |
| lung 25 KC | COPD | 17.02 | 17.02 | lung 25 | |
| asthmatic lung | 29321 | 309.94 | 309.94 | asthmatic | −2.44320675 |
| ODO3112 | | | | lung | |
| asthmatic lung | 29323 | 532.32 | 1064.64 | asthmatic | 1.405933991 |
| ODO3433 | | | | lung | |
| asthmatic lung | 29322 | 1159.05 | 2318.10 | asthmatic | 3.061218426 |
| ODO3397 | | | | lung | |
| asthmatic lung | 29325 | 873.73 | 1747.46 | asthmatic | 2.307647103 |
| ODO4928 | | | | lung | |
| endo cells KC | control | 0 | 0.00 | endo cells | |
| endo VEGF KC | | 0.93 | 0.93 | endo VEGF | 0.93 |
| endo bFGF KC | | 5.16 | 5.16 | endo bFGF | 5.16 |
| heart Clontech | normal | 43.01 | 86.02 | heart | |
| heart (T-1) ischemic | 29417 | 81.55 | 163.10 | heart T-1 | 1.896070681 |
| heart (T-14) non- | 29422 | 51.64 | 103.28 | heart T-14 | 1.200651011 |
| obstructive DCM | | | | | |
| heart (T-3399) DCM | 29426 | 90.27 | 180.54 | heart T-3399 | 2.098814229 |
| adenoid GW99-269 | 26162 | 982.05 | 1964.10 | adenoid | |
| tonsil GW98-280 | 22582 | 3981.71 | 7963.42 | tonsil | |
| T cells PC00314 | 28453 | 265.95 | 531.90 | T cells | |
| PBMNC KC | | 40.89 | 40.89 | PBMNC | |
| monocyte KC | | 62.92 | 125.84 | monocyte | |
| B cells PC00665 | 28455 | 9045.58 | 18091.16 | B cells | |
| dendritic cells 28441 | | 267.47 | 534.94 | dendritic cells | |
| neutrophils | 28440 | 1212.1 | 1212.10 | neutrophils | |
| eosinophils | 28446 | 1563.76 | 3127.52 | eosinophils | |
| BM unstim KC | | 56.55 | 56.55 | BM unstim | |
| BM stim KC | | 27.4 | 27.40 | BM stim | −2.063868613 |
| osteo dif KC | | 0 | 0.00 | osteo dif | |
| osteo undif KC | | 0 | 0.00 | osteo undif | 0 |
| chondrocytes | | 0.92 | 2.30 | chondrocytes | |
| OA Synovium IP12/01 | 29462 | 524.44 | 524.44 | OA | |
| | | | | Synovium | |
| OA Synovium NP10/01 | 29461 | 191.8 | 383.60 | OA | |
| | | | | Synovium | |
| OA Synovium NP57/00 | 28464 | 461.09 | 922.18 | OA | |
| | | | | Synovium | |
| RA Synovium NP03/01 | 28466 | 484.63 | 969.26 | RA Synovium | |
| RA Synovium NP71/00 | 28467 | 698.08 | 1396.16 | RA Synovium | |
| RA Synovium NP45/00 | 28475 | 1034.78 | 2069.56 | RA Synovium | |
| OA bone (biobank) | 29217 | 547.68 | 547.68 | OA bone | |
| | | | | (biobank) | |
| OA bone Sample 1 | J. Emory | 286.6 | 573.20 | OA bone | |
| OA bone Sample 2 | J. Emory | 604.86 | 1209.72 | OA bone | |
| Cartilage (pool) | Normal | 224.68 | 449.36 | Cartilage | |
| | | | | (pool) | |
| Cartilage (pool) | OA | 113.78 | 227.56 | Cartilage | −1.974687994 |
| | | | | (pool) | |
| PBL unifected | 28441 | 966.68 | 1933.36 | PBL | |
| | | | | unifected | |
| PBL HIV IIIB | 28442 | 1353.87 | 2707.74 | PBL HIV | 1.400535855 |
| | | | | IIIB | |
| MRC5 uninfected | 29158 | 1.28 | 2.56 | MRC5 | |
| (100%) | | | | uninfected | |
| | | | | (100%) | |
| MRC5 HSV strain F | 29178 | 34.07 | 68.14 | MRC5 HSV | 26.6171875 |
| | | | | strain F | |
| W12 cells | 29179 | 3.55 | 7.10 | W12cells | |
| Keratinocytes | 29180 | 5.64 | 11.28 | Keratinocytes | |

Gene Name sbg442445PROa

| | Fold Change in Disease |
|---|---|
| | Population Relative to |
| Disease tissues | Normal |
| colon tumor | 1.19 |
| colon tumor | −2.59 |
| colon tumor | −1.93 |
| colon tumor | −1.76 |
| lung tumor | −63.01 |
| lung tumor | 1.66 |
| lung tumor | −2.42 |
| lung tumor | 1.89 |
| breast tumor | 2.48 |
| breast tumor | 15.17 |
| breast tumor | 5.24 |
| breast tumor | −2.63 |
| brain stage 5 ALZ | −1.21 |
| brain stage 5 ALZ | 2.09 |
| brain stage 5 ALZ | 1.45 |
| brain stage 5 ALZ | −2.46 |
| lung 24 | −142.07 |
| lung 28 | −25.04 |
| lung 23 | −14.30 |
| asthmatic lung | −2.44 |
| asthmatic lung | 1.41 |
| asthmatic lung | 3.06 |
| asthmatic lung | 2.31 |
| endo VEGF | 0.93 |
| endo bFGF | 5.16 |
| heart T-1 | 1.90 |
| heart T-14 | 1.20 |
| heart T-3399 | 2.10 |
| BM stim | −2.06 |
| osteo undif | 0.00 |
| Cartilage (pool) | −1.97 |
| PBL HIV IIIB | 1.40 |
| MRC5 HSV strain F | 26.62 |

Gene Name sbg456548CytoRa

Strongly expressed in adenoid/tonsils and dendritic cells. Overexpressed in stimulated bone marrow. Taken together, these data suggest a role in immune function. Expression in GI tract suggests potential role in diseases of the GI system like IBD, Chron's, etc.

| | | | | | | copies of |
|---|---|---|---|---|---|---|
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg456548CytoRa | (sample 1) | (sample 2) | Copies | (ng) | (ng) | RNA |
| Subcutaneous | 0.00 | 5.06 | 2.53 | 3.06 | 16.34 | 41.34 |
| Adipocytes Zenbio | | | | | | |
| Subcutaneous Adipose | 0.00 | 0.00 | 0.00 | 0.96 | 52.36 | 0.00 |
| Zenbio | | | | | | |
| Adrenal Gland Clontech | 0.00 | 0.00 | 0.00 | 0.61 | 81.97 | 0.00 |
| Whole Brain Clontech | 0.00 | 0.00 | 0.00 | 7.24 | 6.91 | 0.00 |
| Fetal Brain Clontech | 0.00 | 0.00 | 0.00 | 0.48 | 103.95 | 0.00 |
| Cerebellum Clontech | 0.00 | 0.00 | 0.00 | 2.17 | 23.04 | 0.00 |
| Cervix | 0.00 | 7.86 | 3.93 | 2.42 | 20.66 | 81.20 |
| Colon | 9.12 | 37.61 | 23.37 | 2.71 | 18.45 | 431.09 |
| Endometrium | 0.00 | 0.00 | 0.00 | 0.73 | 68.21 | 0.00 |
| Esophagus | 0.00 | 0.00 | 0.00 | 1.37 | 36.50 | 0.00 |
| Heart Clontech | 0.00 | 0.00 | 0.00 | 1.32 | 37.88 | 0.00 |
| Hypothalamus | 0.00 | 0.00 | 0.00 | 0.32 | 155.28 | 0.00 |
| Ileum | not done | 39.63 | 39.63 | 2.58 | 19.38 | 768.02 |
| Jejunum | 9.16 | 33.67 | 21.42 | 6.60 | 7.58 | 162.23 |
| Kidney | 0.00 | 0.00 | 0.00 | 2.12 | 23.58 | 0.00 |
| Liver | 0.00 | 13.75 | 6.88 | 1.50 | 33.33 | 229.17 |
| Fetal Liver Clontech | 0.00 | 0.00 | 0.00 | 10.40 | 4.81 | 0.00 |
| Lung | 0.00 | 0.00 | 0.00 | 2.57 | 19.46 | 0.00 |
| Mammary Gland | 136.73 | 106.34 | 121.54 | 13.00 | 3.85 | 467.44 |
| Clontech | | | | | | |
| Myometrium | 27.33 | 17.56 | 22.45 | 2.34 | 21.37 | 479.59 |
| Omentum | 0.00 | 12.61 | 6.31 | 3.94 | 12.69 | 80.01 |
| Ovary | 16.46 | 17.90 | 17.18 | 4.34 | 11.52 | 197.93 |
| Pancreas | 0.00 | 0.00 | 0.00 | 0.81 | 61.80 | 0.00 |
| Head of Pancreas | 0.00 | 0.00 | 0.00 | 1.57 | 31.85 | 0.00 |
| Parotid Gland | 21.25 | 23.72 | 22.49 | 5.48 | 9.12 | 205.16 |
| Placenta Clontech | 101.11 | 73.40 | 87.26 | 5.26 | 9.51 | 829.42 |
| Prostate | 8.55 | 0.00 | 4.28 | 3.00 | 16.67 | 71.25 |
| Rectum | 0.00 | 0.00 | 0.00 | 1.23 | 40.65 | 0.00 |
| Salivary Gland | 0.00 | 0.00 | 0.00 | 7.31 | 6.84 | 0.00 |
| Clontech | | | | | | |
| Skeletal Muscle | 0.00 | 0.00 | 0.00 | 1.26 | 39.68 | 0.00 |
| Clontech | | | | | | |
| Skin | 0.00 | 0.00 | 0.00 | 1.21 | 41.32 | 0.00 |
| Small Intestine | 0.00 | 0.00 | 0.00 | 0.98 | 51.07 | 0.00 |
| Clontech | | | | | | |
| Spleen | 31.60 | 14.66 | 23.13 | 4.92 | 10.16 | 235.06 |
| Stomach | 0.00 | 7.01 | 3.51 | 2.73 | 18.32 | 64.19 |
| Testis Clontech | 0.00 | 0.00 | 0.00 | 0.57 | 87.87 | 0.00 |
| Thymus Clontech | 51.70 | 103.21 | 77.46 | 9.89 | 5.06 | 391.58 |
| Thyroid | 0.00 | 0.00 | 0.00 | 2.77 | 18.05 | 0.00 |
| Trachea Clontech | 0.00 | 0.00 | 0.00 | 9.71 | 5.15 | 0.00 |
| Urinary Bladder | 0.00 | 7.29 | 3.65 | 5.47 | 9.14 | 33.32 |
| Uterus | 5.98 | 21.02 | 13.50 | 5.34 | 9.36 | 126.40 |

| | | | copies of | | |
|---|---|---|---|---|---|
| | Reg | | mRNA | | |
| | number | Mean | detected/50 ng | | Fold Change in |
| Sample | (GSK | GOI | total | | Disease |
| sbg456548CytoRa | identifier) | copies | RNA | Sample | Population |
| colon normal GW98-167 | 21941 | 54.19 | 108.38 | colon normal | |
| colon tumor GW98-166 | 21940 | 242.87 | 485.74 | colon tumor | 4.481823215 |
| colon normal GW98-178 | 22080 | 24.61 | 49.22 | colon normal | |
| colon tumor GW98-177 | 22060 | 17.37 | 34.74 | colon tumor | −1.416810593 |
| colon normal GW98-561 | 23514 | 120.13 | 240.26 | colon normal | |
| colon tumor GW98-560 | 23513 | 43.05 | 86.10 | colon tumor | −2.79047619 |
| colon normal GW98-894 | 24691 | 81.35 | 162.70 | colon normal | |
| colon tumor GW98-893 | 24690 | 16.94 | 33.88 | colon tumor | −4.802243211 |
| lung normal GW98-3 | 20742 | 12.83 | 25.66 | lung normal | |
| lung tumor GW98-2 | 20741 | 94.41 | 188.82 | lung tumor | 7.358534684 |
| lung normal GW97-179 | 20677 | 519.7 | 1039.40 | lung normal | |
| lung tumor GW97-178 | 20676 | 46.83 | 93.66 | lung tumor | −11.09758702 |
| lung normal GW98-165 | 21922 | 7.95 | 15.90 | lung normal | |
| lung tumor GW98-164 | 21921 | 237.54 | 475.08 | lung tumor | 29.87924528 |
| lung normal GW98-282 | 22584 | 251.04 | 502.08 | lung normal | |
| lung tumor GW98-281 | 22583 | 28.16 | 56.32 | lung tumor | −8.914772727 |
| breast normal GW00-392 | 28750 | 138.99 | 138.99 | breast | |
| | | | | normal | |
| breast tumor GW00-391 | 28746 | 147.66 | 295.32 | breast tumor | 2.124757177 |
| breast normal GW00-413 | 28798 | 30.39 | 30.39 | breast | |
| | | | | normal | |
| breast tumor GW00-412 | 28797 | 37.64 | 75.28 | breast tumor | 2.477130635 |
| breast normal GW00- | 27592-95 | 218.09 | 218.09 | breast | |
| 235:238 | | | | normal | |
| breast tumor GW00- | 27588-91 | 14.68 | 14.68 | breast tumor | −14.85626703 |
| 231:234 | | | | | |
| breast normal GW98-621 | 23656 | 1888.3 | 3776.60 | breast | |
| | | | | normal | |
| breast tumor GW98-620 | 23655 | 877.2 | 1754.40 | breast tumor | −2.152644779 |
| brain normal BB99-542 | 25507 | 0 | 0.00 | brain normal | |
| brain normal BB99-406 | 25509 | 0 | 0.00 | brain normal | |
| brain normal BB99-904 | 25546 | 0 | 0.00 | brain normal | |
| brain stage 5 ALZ BB99- | 25502 | 0 | 0.00 | brain stage 5 | 0 |
| 874 | | | | ALZ | |
| brain stage 5 ALZ BB99- | 25503 | 7.32 | 14.64 | brain stage 5 | 14.64 |
| 887 | | | | ALZ | |
| brain stage 5 ALZ BB99- | 25504 | 0 | 0.00 | brain stage 5 | 0 |
| 862 | | | | ALZ | |
| brain stage 5 ALZ BB99- | 25542 | 0 | 0.00 | brain stage 5 | 0 |
| 927 | | | | ALZ | |
| CT lung KC | normal | 10.31 | 20.62 | CT lung | |
| lung 26 KC | normal | 49.79 | 49.79 | lung 26 | |
| lung 27 KC | normal | 4.11 | 4.11 | lung 27 | |
| lung 24 KC | COPD | 0.67 | 0.67 | lung 24 | −38.10074627 |
| lung 28 KC | COPD | 19.24 | 19.24 | lung 28 | −1.326793139 |
| lung 23 KC | COPD | 3.15 | 3.15 | lung 23 | −8.103968254 |
| lung 25 KC | COPD | 27.59 | 27.59 | lung 25 | |
| asthmatic lung | 29321 | 2.95 | 2.95 | asthmatic | −8.653389831 |
| ODO3112 | | | | lung | |
| asthmatic lung | 29323 | 9.86 | 19.72 | asthmatic | −1.294497972 |
| ODO3433 | | | | lung | |
| asthmatic lung | 29322 | 24.39 | 48.78 | asthmatic | 1.910880423 |
| ODO3397 | | | | lung | |
| asthmatic lung | 29325 | 53.84 | 107.68 | asthmatic | 4.218196063 |
| ODO4928 | | | | lung | |
| endo cells KC | control | 0 | 0.00 | endo cells | |
| endo VEGF KC | | 14.65 | 14.65 | endo VEGF | 14.65 |
| endo bFGF KC | | 0 | 0.00 | endo bFGF | 0 |
| heart Clontech | normal | 0 | 0.00 | heart | |
| heart (T-1 ) ischemic | 29417 | 21.18 | 42.36 | heart T-1 | 42.36 |
| heart (T-14) non- | 29422 | 27.4 | 54.80 | heart T-14 | 54.8 |
| obstructive DCM | | | | | |
| heart (T-3399) DCM | 29426 | 93.27 | 186.54 | heart T-3399 | 186.54 |
| adenoid GW99-269 | 26162 | 579.69 | 1159.38 | adenoid | |
| tonsil GW98-280 | 22582 | 3780.08 | 7560.16 | tonsil | |
| T cells PG00314 | 28453 | 5.86 | 11.72 | T cells | |
| PBMNC KC | | 0 | 0.00 | PBMNC | |
| monocyte KC | | 0 | 0.00 | monocyte | |
| B cells PG00665 | 28455 | 19.6 | 39.20 | B cells | |
| dendritic cells 28441 | | 580.67 | 1161.34 | dendritic | |
| | | | | cells | |
| neutrophils | 28440 | 19.76 | 19.76 | neutrophils | |
| eosinophils | 28446 | 15.12 | 30.24 | eosinophils | |
| BM unstim KC | | 0 | 0.00 | BM unstim | |
| BM stim KC | | 296.72 | 296.72 | BM stim | 296.72 |
| osteo dif KC | | 0 | 0.00 | osteo dif | |
| osteo undif KC | | 0 | 0.00 | osteo undif | 0 |
| chondrocytes | | 15.31 | 38.28 | chondrocytes | |
| OA Synovium IP12/01 | 29462 | 39.57 | 39.57 | OA | |
| | | | | Synovium | |
| OA Synovium NP10/01 | 29461 | 0 | 0.00 | OA | |
| | | | | Synovium | |
| OA Synovium NP57/00 | 28464 | 70.08 | 140.16 | OA | |
| | | | | Synovium | |
| RA Synovium NP03/01 | 28466 | 23.73 | 47.46 | RA | |
| | | | | Synovium | |
| RA Synovium NP71/00 | 28467 | 24.13 | 48.26 | RA | |
| | | | | Synovium | |
| RA Synovium NP45/00 | 28475 | 51.88 | 103.76 | RA | |
| | | | | Synovium | |
| OA bone (biobank) | 29217 | 0 | 0.00 | OA bone | |
| | | | | (biobank) | |
| OA bone Sample 1 | J. Emory | 0 | 0.00 | OA bone | |
| OA bone Sample 2 | J. Emory | 5.45 | 10.90 | OA bone | |
| Cartilage (pool) | Normal | 0 | 0.00 | Cartilage | |
| | | | | (pool) | |
| Cartilage (pool) | OA | 0 | 0.00 | Cartilage | 0 |
| | | | | (pool) | |
| PBL unifected | 28441 | 76.67 | 153.34 | PBL | |
| | | | | unifected | |
| PBL HIV IIIB | 28442 | 13.77 | 27.54 | PBL HIV | −5.567901235 |
| | | | | IIIB | |
| MRC5 uninfected | 29158 | 0 | 0.00 | MRC5 | |
| (100%) | | | | uninfected | |
| | | | | (100%) | |
| MRC5 HSV strain F | 29178 | 0 | 0.00 | MRC5 HSV | 0 |
| | | | | strain F | |
| W12 cells | 29179 | 0 | 0.00 | W12 cells | |
| Keratinocytes | 29180 | 0 | 0.00 | Keratinocytes | |

Gene Name sbg456548CytoRa

| | Fold Change in Disease |
|---|---|
| | Population Relative to |
| Disease tissues | Normal |
| colon tumor | 4.48 |
| colon tumor | −1.42 |
| colon tumor | −2.79 |
| colon tumor | −4.80 |
| lung tumor | 7.36 |

Gene Name sbg442358PROa

Expression in multiple immune cell types as well as stimulated bone marrow and thymus strongly suggests function in immune system. Overexpressed in breast tumors (1/4). Expression in RA and OA with corroborating expression in immune cells suggests role in these diseases. Overexpressed in heart disease suggesting role in CV diseases. Downregulated in HSV infected cells suggesting possible host cell factor.

| | | | | | | copies |
|---|---|---|---|---|---|---|
| | | | | | | of |
| | | | | | | mRNA |
| | | | | | | detected/ |
| | Mean GOI | Mean GOI | Average | 18S | 50 ng/18S | 50 ng |
| Sample | copies | copies | GOI | rRNA | rRNA | total |
| sbg442358PROa | (sample 1) | (sample 2) | Copies | (ng) | (ng) | RNA |
| Subcutaneous | 1.86 | 1.71 | 1.79 | 3.06 | 16.34 | 29.17 |
| Adipocytes Zenbio | | | | | | |
| Subcutaneous Adipose | 0.71 | 0.73 | 0.72 | 0.96 | 52.36 | 37.70 |
| Zenbio | | | | | | |
| Adrenal Gland Clontech | 3.45 | 1.89 | 2.67 | 0.61 | 81.97 | 218.85 |
| Whole Brain Clontech | 406.27 | 496.60 | 451.44 | 7.24 | 6.91 | 3117.65 |
| Fetal Brain Clontech | 3.82 | 1.68 | 2.75 | 0.48 | 103.95 | 285.86 |
| Cerebellum Clontech | 5.84 | 30.51 | 18.18 | 2.17 | 23.04 | 418.78 |
| Cervix | 2.50 | 0.48 | 1.49 | 2.42 | 20.66 | 30.79 |
| Colon | 18.45 | 18.77 | 18.61 | 2.71 | 18.45 | 343.36 |
| Endometrium | 4.93 | 0.30 | 2.62 | 0.73 | 68.21 | 178.38 |
| Esophagus | 8.97 | 6.99 | 7.98 | 1.37 | 36.50 | 291.24 |
| Heart Clontech | 5.26 | 16.53 | 10.90 | 1.32 | 37.88 | 412.69 |
| Hypothalamus | 2.10 | 2.41 | 2.26 | 0.32 | 155.28 | 350.16 |
| Ileum | 18.94 | 12.62 | 15.78 | 2.58 | 19.38 | 305.81 |
| Jejunum | 65.51 | 95.24 | 80.38 | 6.60 | 7.58 | 608.90 |
| Kidney | 2.60 | 3.81 | 3.21 | 2.12 | 23.58 | 75.59 |
| Liver | 7.19 | 7.05 | 7.12 | 1.50 | 33.33 | 237.33 |
| Fetal Liver Clontech | 1252.22 | 1363.06 | 1307.64 | 10.40 | 4.81 | 6286.73 |
| Lung | 27.57 | 6.97 | 17.27 | 2.57 | 19.46 | 335.99 |
| Mammary Gland | 79.83 | 72.99 | 76.41 | 13.00 | 3.85 | 293.88 |
| Clontech | | | | | | |
| Myometrium | 2.46 | 10.62 | 6.54 | 2.34 | 21.37 | 139.74 |
| Omentum | 10.40 | 3.27 | 6.84 | 3.94 | 12.69 | 86.74 |
| Ovary | 17.71 | 31.15 | 24.43 | 4.34 | 11.52 | 281.45 |
| Pancreas | 3.33 | 1.74 | 2.54 | 0.81 | 61.80 | 156.67 |
| Head of Pancreas | 3.82 | 6.17 | 5.00 | 1.57 | 31.85 | 159.08 |
| Parotid Gland | 22.77 | 22.54 | 22.66 | 5.48 | 9.12 | 206.71 |
| Placenta Clontech | 14.71 | 53.83 | 34.27 | 5.26 | 9.51 | 325.76 |
| Prostate | 16.71 | 19.39 | 18.05 | 3.00 | 16.67 | 300.83 |
| Rectum | 6.71 | 3.49 | 5.10 | 1.23 | 40.65 | 207.32 |
| Salivary Gland | 55.38 | 9.30 | 32.34 | 7.31 | 6.84 | 221.20 |
| Clontech | | | | | | |
| Skeletal Muscle | 3.79 | 4.16 | 3.98 | 1.26 | 39.68 | 157.74 |
| Clontech | | | | | | |
| Skin | 4.51 | 14.47 | 9.49 | 1.21 | 41.32 | 392.15 |
| Small Intestine | 8.12 | 7.87 | 8.00 | 0.98 | 51.07 | 408.32 |
| Clontech | | | | | | |
| Spleen | 14.88 | 17.12 | 16.00 | 4.92 | 10.16 | 162.60 |
| Stomach | 21.85 | 11.68 | 16.77 | 2.73 | 18.32 | 307.05 |
| Testis Clontech | 22.77 | 11.54 | 17.16 | 0.57 | 87.87 | 1507.47 |
| Thymus Clontech | 1990.82 | 1374.71 | 1682.77 | 9.89 | 5.06 | 8507.41 |
| Thyroid | 16.85 | 2.86 | 9.86 | 2.77 | 18.05 | 177.89 |
| Trachea Clontech | 29.69 | 82.85 | 56.27 | 9.71 | 5.15 | 289.75 |
| Urinary Bladder | 2.32 | 13.42 | 7.87 | 5.47 | 9.14 | 71.94 |
| Uterus | 8.86 | 11.18 | 10.02 | 5.34 | 9.36 | 93.82 |

| | | | copies of | | |
|---|---|---|---|---|---|
| | Reg | | mRNA | | |
| | number | Mean | detected/50 ng | | Fold Change |
| Sample | (GSK | GOI | total | | in Disease |
| sbg442358PROa | identifier) | copies | RNA | Sample | Population |
| colon normal GW98-167 | 21941 | 1232.32 | 2464.64 | colon normal | |
| colon tumor GW98-166 | 21940 | 2940.17 | 5880.34 | colon tumor | 2.385881914 |
| colon normal GW98-178 | 22080 | 221.26 | 442.52 | colon normal | |
| colon tumor GW98-177 | 22060 | 709.52 | 1419.04 | colon tumor | 3.20672512 |
| colon normal GW98-561 | 23514 | 985.52 | 1971.04 | colon normal | |
| colon tumor GW98-560 | 23513 | 829.67 | 1659.34 | colon tumor | −1.18784577 |
| colon normal GW98-894 | 24691 | 2738.17 | 5476.34 | colon normal | |
| colon tumor GW98-893 | 24690 | 3022.06 | 6044.12 | colon tumor | 1.103678734 |
| lung normal GW98-3 | 20742 | 536.82 | 1073.64 | lung normal | |
| lung tumor GW98-2 | 20741 | 594.2 | 1188.40 | lung tumor | 1.106888715 |
| lung normal GW97-179 | 20677 | 4382.61 | 8765.22 | lung normal | |
| lung tumor GW97-178 | 20676 | 359.07 | 718.14 | lung tumor | −12.20544741 |
| lung normal GW98-165 | 21922 | 622.06 | 1244.12 | lung normal | |
| lung tumor GW98-164 | 21921 | 1299.85 | 2599.70 | lung tumor | 2.089589429 |
| lung normal GW98-282 | 22584 | 1782.09 | 3564.18 | lung normal | |
| lung tumor GW98-281 | 22583 | 470.51 | 941.02 | lung tumor | −3.787570934 |
| breast normal GW00-392 | 28750 | 429 | 429.00 | breast normal | |
| breast tumor GW00-391 | 28746 | 417.99 | 835.98 | breast tumor | 1.948671329 |
| breast normal GW00-413 | 28798 | 16.03 | 16.03 | breast normal | |
| breast tumor GW00-412 | 28797 | 1048.11 | 2096.22 | breast tumor | 130.768559 |
| breast normal GW00- | 27592-95 | 2.17 | 2.17 | breast normal | |
| 235:238 | | | | | |
| breast tumor GW00- | 27588-91 | 69.91 | 69.91 | breast tumor | 32.21658986 |
| 231:234 | | | | | |
| breast normal GW98-621 | 23656 | 1037.08 | 2074.16 | breast normal | |
| breast tumor GW98-620 | 23655 | 1010.59 | 2021.18 | breast tumor | −1.026212411 |
| brain normal BB99-542 | 25507 | 299.28 | 598.56 | brain normal | |
| brain normal BB99-406 | 25509 | 250.85 | 501.70 | brain normal | |
| brain normal BB99-904 | 25546 | 97.7 | 195.40 | brain normal | |
| brain stage 5 ALZ BB99- | 25502 | 125 | 250.00 | brain stage 5 | −1.727546667 |
| 874 | | | | ALZ | |
| brain stage 5 ALZ BB99- | 25503 | 850.01 | 1700.02 | brain stage 5 | 3.936264143 |
| 887 | | | | ALZ | |
| brain stage 5 ALZ BB99- | 25504 | 347.91 | 695.82 | brain stage 5 | 1.611117114 |
| 862 | | | | ALZ | |
| brain stage 5 ALZ BB99- | 25542 | 147.11 | 294.22 | brain stage 5 | −1.467903836 |
| 927 | | | | ALZ | |
| CT lung KC | normal | 130.37 | 260.74 | CT lung | |
| lung 26 KC | normal | 159.19 | 159.19 | lung 26 | |
| lung 27 KC | normal | 0.49 | 0.49 | lung 27 | |
| lung 24 KC | COPD | 2.37 | 2.37 | lung 24 | −47.89873418 |
| lung 28 KC | COPD | 45.72 | 45.72 | lung 28 | −2.482939633 |
| lung 23 KC | COPD | 20.36 | 20.36 | lung 23 | −5.575638507 |
| lung 25 KC | COPD | 33.66 | 33.66 | lung 25 | |
| asthmatic lung | 29321 | 65.46 | 65.46 | asthmatic | −1.734188818 |
| ODO3112 | | | | lung | |
| asthmatic lung | 29323 | 532.42 | 1064.84 | asthmatic | 9.380197322 |
| ODO3433 | | | | lung | |
| asthmatic lung | 29322 | 2865.67 | 5731.34 | asthmatic | 50.48749119 |
| ODO3397 | | | | lung | |
| asthmatic lung | 29325 | 494.27 | 988.54 | asthmatic | 8.708069063 |
| ODO4928 | | | | lung | |
| endo cells KC | control | 62.77 | 62.77 | endo cells | |
| endo VEGF KC | | 22.41 | 22.41 | endo VEGF | −2.800981705 |
| endo bFGF KC | | 33.16 | 33.16 | endo bFGF | −1.892943305 |
| heart Clontech | normal | 74.18 | 148.36 | heart | |
| heart ( T-1 ) ischemic | 29417 | 270.07 | 540.14 | heart T-1 | 3.640738744 |
| heart (T-14) non- | 29422 | 680.12 | 1360.24 | heart T-14 | 9.168509032 |
| obstructive DCM | | | | | |
| heart (T-3399) DCM | 29426 | 414 | 828.00 | heart T-3399 | 5.581019143 |
| adenoid GW99-269 | 26162 | 781.46 | 1562.92 | adenoid | |
| tonsil GW98-280 | 22582 | 2279.13 | 4558.26 | tonsil | |
| T cells PC00314 | 28453 | 1129.27 | 2258.54 | T cells | |
| PBMNC KC | | 27.98 | 27.98 | PBMNC | |
| monocyte KC | | 3.55 | 7.10 | monocyte | |
| B cells PC00665 | 28455 | 872.58 | 1745.16 | B cells | |
| dendritic cells 28441 | | 1055.22 | 2110.44 | dendritic cells | |
| neutrophils | 28440 | 740.39 | 740.39 | neutrophils | |
| eosinophils | 28446 | 1081.83 | 2163.66 | eosinophils | |
| BM unstim KC | | 50.91 | 50.91 | BM unstim | |
| BM stim KC | | 391.11 | 391.11 | BM stim | 7.682380672 |
| osteo dif KC | | 161.31 | 161.31 | osteo dif | |
| osteo undif KC | | 40.01 | 40.01 | osteo undif | −4.031742064 |
| chondrocytes | | 2250.59 | 5626.48 | chondrocytes | |
| OA Synovium IP12/01 | 29462 | 229.19 | 229.19 | OA | |
| | | | | Synovium | |
| OA Synovium NP10/01 | 29461 | 152.3 | 304.60 | OA | |
| | | | | Synovium | |
| OA Synovium NP57/00 | 28464 | 413.06 | 826.12 | OA | |
| | | | | Synovium | |
| RA Synovium NP03/01 | 28466 | 611.02 | 1222.04 | RA Synovium | |
| RA Synovium NP71/00 | 28467 | 385.94 | 771.88 | RA Synovium | |
| RA Synovium NP45/00 | 28475 | 1701.68 | 3403.36 | RA Synovium | |
| OA bone (biobank) | 29217 | 225.69 | 225.69 | OA bone | |
| | | | | (biobank) | |
| OA bone Sample 1 | J. Emory | 306.63 | 613.26 | OA bone | |
| OA bone Sample 2 | J. Emory | 1811.32 | 3622.64 | OA bone | |
| Cartilage (pool) | Normal | 384.44 | 768.88 | Cartilage | |
| | | | | (pool) | |
| Cartilage (pool) | OA | 174.53 | 349.06 | Cartilage | −2.202715865 |
| | | | | (pool) | |
| PBL unifected | 28441 | 9016.82 | 18033.64 | PBL | |
| | | | | unifected | |
| PBL HIV IIIB | 28442 | 4331.76 | 8663.52 | PBL HIV | −2.081560382 |
| | | | | IIIB | |
| MRC5 uninfected | 29158 | 2232.48 | 4464.96 | MRC5 | |
| (100%) | | | | uninfected | |
| | | | | (100%) | |
| MRC5 HSV strain F | 29178 | 419.67 | 839.34 | MRC5 HSV | −5.319608264 |
| | | | | strain F | |
| W12 cells | 29179 | 3336.07 | 6672.14 | W12 cells | |
| Keratinocytes | 29180 | 5568.91 | 11137.82 | Keratinocytes | |

Gene Name sbg442358PROa

| | Fold Change in Disease |
|---|---|
| | Population Relative to |
| Disease tissues | Normal |
| colon tumor | 2.39 |
| colon tumor | 3.21 |
| colon tumor | −1.19 |
| colon tumor | 1.10 |
| lung tumor | 1.11 |
| lung tumor | −12.21 |
| lung tumor | 2.09 |
| lung tumor | −3.79 |
| breast tumor | 1.95 |
| breast tumor | 130.77 |
| breast tumor | 32.22 |
| breast tumor | −1.03 |
| brain stage 5 ALZ | −1.73 |
| brain stage 5 ALZ | 3.94 |
| brain stage 5 ALZ | 1.61 |
| brain stage 5 ALZ | −1.47 |
| lung 24 | −47.90 |
| lung 28 | −2.48 |
| lung 23 | −5.58 |
| asthmatic lung | −1.73 |
| asthmatic lung | 9.38 |
| asthmatic lung | 50.49 |
| asthmatic lung | 8.71 |
| endo VEGF | −2.80 |
| endo bFGF | −1.89 |
| heart T-1 | 3.64 |
| heart T-14 | 9.17 |
| heart T-3399 | 5.58 |
| BM stim | 7.68 |
| osteo undif | −4.03 |
| Cartilage (pool) | −2.20 |
| PBL HIV IIIB | −2.08 |
| MRC5 HSV strain F | −5.32 |

TABLE V
Additional diseases based on mRNA expression in specific tissues

| Tissue | |
|---|---|
| Expression | Additional Diseases |
| Brain | Neurological and psychiatric diseases, including Alzheimers, parasupranuclear |
| | palsey, Huntington's disease, myotonic dystrophy, anorexia, depression, |
| | schizophrenia, headache, amnesias, anxiety disorders, sleep disorders, multiple |
| | sclerosis |
| Heart | Cardiovascular diseases, including congestive heart failure, dilated |
| | cardiomyopathy, cardiac arrhythmias, Hodgson's Disease, myocardial |
| | infarction, cardiac arrhythmias |
| Lung | Respiratory diseases, including asthma, Chronic Obstructive Pulmonary |
| | Disease, cystic fibrosis, acute bronchitis, adult respiratory distress syndrome |
| Liver | Dyslipidemia, hypercholesterolemia, hypertriglyceridemia, cirrhosis, hepatic |
| | encephalopatby, fatty hepatocirrhosis, viral and nonviral hepatitis, Type II |
| | Diabetes Mellitis, impaired glucose tolerance |
| Kidney | Renal diseases, including acute and chronic renal failure, acute tubular necrosis, |
| | cystinuria, Fanconi's Syndrome, glomerulonephritis, renal cell carcinoma, |
| | renovascular hypertension |
| Skeletal | Eulenburg's Disease, hypoglycemia, obesity, tendinitis, periodic paralyses, |
| muscle | malignant hyperthermia, paramyotonia congenita, myotonia congenita |
| Intestine | Gastrointestinal diseases, including Myotonia congenita, Ileus, Intestinal |
| | Obstruction, Tropical Sprue, Pseudomembranous Enterocolitis |
| Spleen/lymph | Lymphangiectasia, hypersplenism, angiomas, ankylosing spondylitis, Hodgkin's |
| | Disease, macroglobulinemia, malignant lymphomas, rheumatoid arthritis |
| Placenta | Choriocarcinoma, hydatidiform mole, placenta previa |
| Testis | Testicular cancer, male reproductive diseases, including low testosterone and |
| | male infertility |
| Pancreas | Diabetic ketoacidosis, Type 1 & 2 diabetes, obesity, impaired glucose tolerance |

What is claimed is:

1. An isolated polypeptide selected from the group consisting of:
(a) an isolated polypeptide encoded by a polynucleotide comprising a sequence set forth in Table I;
(b) an isolated polypeptide comprising a polypeptide sequence set forth in Table I; and
(c) a polypeptide sequence of a gene set forth in Table I.

2. An isolated polynucleotide selected from the group consisting of:
(a) an isolated polynucleotide comprising a polynucleotide sequence set forth in Table I;
(b) an isolated polynucleotide of a gene set forth in Table I;
(c) an isolated polynucleotide comprising a polynucleotide sequence encoding a polypeptide set forth in Table I;
(d) an isolated polynucleotide encoding a polypeptide set forth in Table I;
(e) a polynucleotide which is an RNA equivalent of the polynucleotide of (a) to (d);
or a polynucleotide sequence complementary to said isolated polynucleotide.

3. An expression vector comprising a polynucleotide capable of producing a polypeptide of claim 1 when said expression vector is present in a compatible host cell.

4. A process for producing a recombinant host cell which comprises the step of introducing an expression vector comprising a polynucleotide capable of producing a polypeptide of claim 1 into a cell such that the host cell, under appropriate culture conditions, produces said polypeptide.

5. A recombinant host cell produced by the process of claim 4.

6. A membrane of a recombinant host cell of claim 5 expressing said polypeptide.

7. A process for producing a polypeptide which comprises culturing a host cell of claim 5 under conditions sufficient for the production of said polypeptide and recovering said polypeptide from the culture.
